(19) United States
(12) Patent Application Publication
(10) Pub. No.: US 2014/0005165 A1
(43) Pub. Date: Jan. 2, 2014

(54) SUBSTITUTED 3-AZABICYCLO[3.1.0]HEXANE DERIVATIVES USEFUL AS CCR2 ANTAGONISTS

(71) Applicants: Anilkumar G. Nair, Edison, NJ (US); Jose S. Duca, Hopkinton, MA (US); Michael P. Dwyer, Scotch Plains, NJ (US); Joseph A. Kozlowski, Princeton, NJ (US); Stuart B. Rosenblum, West Orange, NJ (US)

(72) Inventors: Anilkumar G. Nair, Edison, NJ (US); Jose S. Duca, Hopkinton, MA (US); Michael P. Dwyer, Scotch Plains, NJ (US); Joseph A. Kozlowski, Princeton, NJ (US); Stuart B. Rosenblum, West Orange, NJ (US)

(21) Appl. No.: 14/004,449

(22) PCT Filed: Mar. 14, 2012
(86) PCT No.: PCT/US12/28969

§ 371 Date: Sep. 11, 2013

Related U.S. Application Data
(60) Provisional application No. 61/453,634, filed on Mar. 17, 2011.

(51) Int. Cl. C07D 209/52; C07D 409/14; C07D 405/04; C07D 403/12; C07D 403/14; C07D 403/08; C07D 471/04; C07D 401/08; C07D 417/08; C07D 413/04; C07D 409/04

(52) U.S. Cl. CPC C07D 209/52; C07D 413/04; C07D 409/14; C07D 405/04; C07D 403/12; C07D 409/04; C07D 403/08; C07D 471/04; C07D 401/08; C07D 417/08; C07D 403/14
USPC 514/210.18; 548/515; 514/412; 548/145; 514/364; 548/468; 514/414; 548/305.1; 514/394; 548/465; 548/364.7; 514/406; 546/122; 514/300; 546/276.7; 514/343; 544/333; 514/256; 548/204; 514/365; 544/331; 514/275; 544/316; 514/274

(57) ABSTRACT

Disclosed are CCR2 antagonists of Formula (I) or pharmaceutically acceptable thereof, wherein R1, L1 and A are defined herein. Also disclosed are pharmaceutical compositions containing the compounds, methods of treatment using the compounds, and compositions to treat diseases or disorders associated with CCR2 activity.

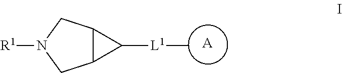

### FIELD OF THE INVENTION

The present invention relates to novel compounds useful as CCR2 antagonists or modulators, pharmaceutical compositions containing the compounds and methods of treatment using the compounds, and compositions to treat diseases or disorders associated with CCR2 activity.

### BACKGROUND OF THE INVENTION

Inflammation is a complex response of vascularized tissues to harmful signals such as pathogens, injured cells or irritants. During inflammation leukocytes migrate into the inflamed tissue to start the healing process. This complex process is modulated by adhesion molecules and chemoattractants. Inflammation also plays a role in diseases such as hay fever, rheumatoid arthritis, inflammatory bowel disease, multiple sclerosis and in atherosclerosis. As a result, there has been increased interest in the molecules involved in the inflammation response, including chemokines and their receptors, as potential drug targets for the management of such disorders.

Chemokines are a group of cytokines made up of 70 to 120 amino acid residues. They are broadly classified based on function as inflammatory and/or homeostatic. Inflammatory chemokines are induced during an immune response to promote cells of the immune system to a site of infection, tissue damage or other physiological abnormalities. Induction is triggered by tumor necrosis factor, interferon-gamma, microbial products, and trauma. Inflammatory chemokines are expressed by circulating leukocytes and other cells upon activation. Homeostatic chemokines are involved in cell migration during tissue maintenance or development and are expressed locally. (Handel, Annu. Rev. Immunol., 25, 787-820 (2007)).

Chemokines are also classified structurally based on the number and spacing of the N-terminal cysteine residues in the peptide sequence. There are four groups namely, C (gamma-chemokine), CC (beta-chemokine), CXC (alpha-chemokine) and CX3C (delta-chemokine). Alpha-chemokines, such as interleukin-8 (IL-8), neutrohil-activating protein-2 (NAP-2) and melanoma growth-activating protein (MGSA) are chemoattractants primarily to neutrophils, and beta-chemokines such as RANTES, MIP-1alpha, MP-1beta, monocyte chemotactic protein-1 (MCP-1), MCP-2, MCP-3 and eotaxin are chemoattractants for macrophates, monocytes, T-cells, eosinophils and basophils (Deng, et al., Nature, 381, 661-666 (1996)). The gamma-chemokine such as lymphotactin (alpha and beta) attract T-cell precursors. (Giancarlo, et al., Eur. J. Immunol., 26, 3238-3241 (1996)). There is only one delta-chemokine discovered, namely fractalkine which is a dual chemoattractant and adhesion molecule. (Murphy, et al., Rheumatology, 47, 1446-1451 (2008)).

Chemokine receptors form a sub-family of G-protein coupled receptors (GPCR's) which consists of at least fifteen members. All of these receptors are made up of seven helical membrane-spanning regions connected by extra-membrane loops. The chemokine receptors interact with a number of chemokines and most chemokines interact with more than one receptor. When a chemokine binds to its receptor a complex network of intracellular signaling pathways is activated involving secondary messengers such as calcium, cAMP and phospholipids, resulting in a number of responses such as changes in cell shape, increased expression of cellular adhesion molecules, degranulation, and promotion of cell migration (Allen, et al., Annu. Rev. Immunol., 25, 787-820 (2007)). Specific chemokine receptors, among others, include CCR1, CCR2, CCR2a, CCR2B, CCR3, CCR4, CCR5, CCR7, CXCR3, CXCR-4, CXCR-5, XCR1, and CX3CR1 (Zlotnik and Yoshie, Immunity, 12, 121-127 (2007)).

Chemokines and chemokine receptors in addition to playing a role in the immune response, are also involved in autoimmune disorders (e.g., psoriasis, rheumatoid arthritis, and multiple sclerosis), pulmonary diseases (e.g., asthma and chronic obstructive pulmonary disease), transplant rejection, cancer, HIV infection, and vascular diseases (e.g., atherosclerosis). (Allen, et al., Annu. Rev. Immunol., 25, 787-820 (2007)).

MCP-1 is a well characterized chemokine whose primary receptor is CCR2. Upon binding of MCP-1 to CCR2, there is a rapid increase in calcium concentration, an increase in the expression of cellular adhesion molecules, cellular degranulation is induced, and leukocyte migration is promoted.

In a study, MCP-1^{-/-} and mice were unable to recruit monocytes into sites of inflammation after exposure to thioglycollate, even though their leukocyte and monocyte levels were normal (Lu, et al., J. Exp. Med., 187, 601-608 (1998)). CCR2^{-/-} mice were also unable to recruit monocytes and leukocytes when exposed to thioglycollate and Listeria monocytogenes. (Boring, et al., J. Clin. Invest. 100, 2552-2561 (1997); Kurihara, et al., J. Exp. Med., 186, 1757-1762 (1997)). MCP-1^{-/-} and CCR2^{-/-} mice were found to develop normally relative to the wild-type. This data suggests that antagonism of MCP-1 and/or CCR2 plays a major role in inflammation and would be useful in treating inflammatory and autoimmune disorders.

MCP-1 is over expressed in the synovial tissue of rheumatoid arthritis patients. A MCP-1 antagonist was shown to prevent the onset of rheumatoid arthritis and to reduce disease symptoms after onset of the disease (Gong, et al., J. Exp. Med., 186, 131-137 (1997)). A DNA vaccine encoding MCP-1 was shown to inhibit the development and progression of chronic polyadjuvant-induced arthritis (Youssef, et al., J. Clin. Invest., 106, 361-371 (2000)). Administration of anti-MCP-1 to rats was shown to reduce ankle edema and T cell migration rats with Streptococcal cell wall-induced arthritis (Schimmer, et al., J. Immun., 160, 1466-1471 (1998)). Similar results were seen in a similar study with collagen-induced arthritis in rats (Ogata, et al., J. Pathol., 182, 106-114 (1997)). This data demonstrates the potential for MCP-1 or CCR2 antagonism for the treatment of rheumatoid arthritis.

MCP-1 also plays a role in atherogenesis. In one study, MCP-1 was shown to be expressed in higher levels in atherosclerotic lesions over normal tissue (Nelken, et al., J. Clin. Invest., 88, 1121-1127 (1991)). Mice possessing the CCR2^{-/-} genotype exhibited lower atherosclerotic lesion formation over those the CCR2^{+/+} genotype (Boring, et al., Nature, 394, 894-897 (1998)). In another study, LDL-R^{-/-}/MCP-1^{-/-} mice exhibited significantly less lipid deposition in the aorta over LDL-R^{-/-}/MCP-1^{+/+} mice (Gu, et al., Molecular Cell, 2, 275-281 (1998)). These studies demonstrate the potential of MCP-1 or CCR2 antagonism for the treatment of atherosclerosis.

Other studies have demonstrated the potential use of MCP-1 or CCR2 antagonism for treatment of diseases such as multiple sclerosis (Kennedy, et al., J. Neuroimmunol., 92, 98-108 (1998); Fife, et al., J. Exp. Med., 192, 899 (2000)), bronchiolitis obliterans syndrome (Belperio, et al., J. Clin. Invest., 108, 547-556 (2001)), asthma (Gonzalo, et al., J. Exp. Med., 188, 157-167 (1998), Lukacs, et al., J. Immunol. 158, 4398-4404 (1997), Lu, et al., J. Exp. Med., 187, 601-608 (1998)), kidney disease (Lloyd, et al., J. Exp. Med, 185, 1371-1380 (1997); Tesch, et al., J. Clin. Invest., 103, 73-80 (1999)), colitis (Andres, et al., J. Immunol., 164, 6303-6312 (2000)), alveolitis (Jones, et al., J. Immunol., 149, 2147-2154 (1992)), cancer (Saleedo, et al., Blood, 96, 34-40 (2000)), restinosis (Roque, et al., Arterioscler. Thromb. Vasc. Biol., 22, 554-559 (2002)), HIV infection (Smith, et al., Science, 277, 959-965 (1997)).

A number of patent/patent publications such as WO2009/076404, WO2008/145681, WO2008/070301, WO2008/109238, WO2008/045564, WO2008/008375, WO2007/147026, WO2007/130712, WO2007/014008, WO2006/013427, WO2004/098516, WO2004/050024, WO2005/060665, WO2004/069810, WO2004/041279, WO2004/041161, WO2004/041163, WO2004/041777, WO2004/082616, WO2003/093266, WO2003/092586, WO2001/057226, US2002106369, WO2002/72549, WO2002/070523, WO2002/079151, WO2004/097425, and WO2003/089004 disclose modulators of CCR2 that are useful for treating diseases or disorders such as autoimmune and inflammatory diseases, HIV infection, cancer, atherosclerosis, restenosis, organ transplant rejection, lung fibrosis, rheumatoid arthritis, stenosis, asthma, and tumor relapse.

Accordingly, CCR2 antagonism is an attractive target for the discovery of novel chemotherapeutics. There is a need for compounds useful as CCR2 antagonists to be used alone or in combination in the treatment diseases or disorders such as autoimmune and inflammatory diseases, HIV infection, cancer, atherosclerosis, restenosis, organ transplant rejection, lung fibrosis, rheumatoid arthritis, stenosis, asthma, and tumor relapse.

### SUMMARY OF THE INVENTION

In its many embodiments, the present invention provides a novel class of 3-azabicyclo[3.1.0]hexane derivatives that are antagonists of CCR2, or metabolites, stereoisomers, salts, solvates or polymorphs thereof, methods of preparing such compounds, pharmaceutical compositions comprising one or more such compounds, methods of preparing pharmaceutical formulations comprising one or more such compounds, and methods of treatment, prevention, inhibition or amelioration of one or more conditions associated with CCR2 using such compounds or pharmaceutical compositions. Conditions associated with CCR2 include certain autoimmune and inflammatory diseases, HIV infection, cancer, atherosclerosis, restenosis, organ transplant rejection, lung fibrosis, rheumatoid arthritis, stenosis, asthma, and tumor relapse.

In one embodiment, the present application discloses a compound, or pharmaceutically acceptable salt thereof, of said compound, said compounds having the general structure shown in Formula I below:

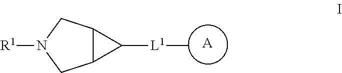

wherein:

L¹ is selected from the group consisting of:
(a) —C(O)—,
(b) —C(O)-heterocyclyl-,
(c) —C(O)-heterocyclyl-O—,
(d) —C(O)-heterocyclyl-N(R⁴)—,
(e) —C(O)-heterocyclyl-N(R⁴)C(O)—,
(f) —C(O)N(R⁴)—,
(g) —C(O)N(R⁴)—C₁₋₆alkylenyl-,
(h) —C(O)N(R⁴)—C₁₋₆alkylenyl-O—,
(i) —C(O)N(R⁴)—C₁₋₆alkylenyl-C₃₋₈cycloalkyl-,
(j) —C(O)N(R⁴)—C₁₋₆alkylenyl-C(O)—N(R⁴)—,
(k) —C(O)N(R⁴)-heterocyclyl-,
(l) —C(O)N(R⁴)-heterocyclyl-C(O)—, and
(m) 1,3,4-oxadiazolyl,
   wherein said heterocyclyl or cycloalkyl groups are unsubstituted or substituted with 1-3 R⁶ groups, and wherein heterocyclyl is a 4-7-membered ring containing 1-2 N heteroatoms;

Ring A is selected from the group consisting of:
(a) aryl,
(b) C₃₋₈cycloalkyl,
(c) C₃₋₈cycloalkenyl,
(d) 5 or 6-membered heterocyclyl,
(e) 5 or 6-membered heterocyclenyl,
(f) 5 or 6-membered monocyclic heteroaryl,
(g) 9- or 10-membered cycloalkylaryl,
(h) 9- or 10-membered heterocyclylheteroaryl,
(i) 9- or 10-membered heterocyclylaryl, and
(j) 9- or 10-membered heteroarylaryl,
   wherein said heterocyclyl, heterocyclenyl, heterocyclylheteroaryl, heterocyclylaryl, heteroarylaryl or heteroaryl rings have 1-4 heteroatoms selected from the group consisting of O, N, and S; wherein said aryl, cycloalkyl, cycloalkenyl, heterocyclyl, heterocyclenyl, heteroaryl, 9- or 10-membered cycloalkylaryl, 9- or 10-membered heterocyclylheteroaryl, 9- or 10-membered heterocyclylaryl, or 9- or 10-membered heteroarylaryl are unsubstituted or substituted with 1-3 R⁵ groups.

R¹ is selected from the group consisting of:
(a) C₁₋₆alkyl,
(b) C₃₋₈cycloalkyl,
(c) C₃₋₈cycloalkenyl,
(d) 5 or 6-membered heterocyclyl, and
(e) 7- or 8-membered bicycloalkyl,
   wherein said alkyl, cycloalkyl, cylcoalkenyl, heterocyclyl, and bicycloalkyl groups are unsubstituted or substituted with 1-3 R² groups, wherein said heterocyclyl, or heterocyclenyl rings have 1-4 heteroatoms selected from the group consisting of O, N, and S;

each R² is independently selected from the group consisting of
(a) hydrogen,
(b) HO—,
(c) halo,
(d) C₁₋₆alkyl,
(e) —C₁₋₆alkyl-OH,
(f) —O—C₁₋₆alkyl,
(g) aryl-C₁₋₆alkyl-O—C₁₋₆alkyl-,
(h) C₂₋₆alkenyl,
(i) oxo,
(j) —NH—C(O)—C₁₋₆alkyl,
(k) —CO₂—C₁₋₆alkyl,
(l) —NH—CO₂—C₁₋₆alkyl,
(m) —NH—C(O)-aryl,
(n) —NH—SO₂—C₃₋₈cycloalkyl,
(o) —NH-aryl,
(p) aryl,
(q) aryl-C₁₋₆alkyl-,
(r) C₃₋₈cycloalkyl,
(s) 5-7-membered heterocyclyl,
(t) 5-7-membered heterocyclenyl,
(u) 5 or 6-membered monocyclic heteroaryl, and
(v) 9-membered heteroarylaryl,
   wherein said alkyl, aryl, cycloalkyl, heterocyclyl, heterocyclenyl and heteroaryl groups are unsubstituted or substituted with 1-3 R³ groups, and wherein said heterocyclyl, heterocyclenyl, or heteroaryl rings have 1-4 heteroatoms selected from the group consisting of O, N and S;

each R³ is independently selected from the group consisting of:
(a) oxo,
(b) —OH,
(c) halo,
(d) C₁₋₆alkyl,
(e) aryl,
(f) aryl-C₁₋₆alkyl-,
(g) C₁₋₆alkoxyl,
(h) aryl-C₁₋₆alkyl-O—, and
(i) cyano;

R⁴ is selected from the group consisting of:
(a) hydrogen,
(b) C₁₋₆alkyl, and
(c) CF₃-phenyl-C₁₋₃alkyl-;

each R⁵ is independently selected from the group consisting of:
(a) oxo,
(b) —OH,
(c) halo,
(d) C₁₋₆alkyl,
(e) C₁₋₆haloalkyl,
(f) aryl,
(g) aryl-C₁₋₆alkyl-,
(h) C₁₋₆alkoxyl,
(i) aryl-C₁₋₆alkyl-O—, and
(j) cyano;
(k) F₅S—; and

each R⁶ is independently selected from the group consisting of
(a) hydrogen,
(b) bromo,
(c) chloro,
(d) fluoro,
(e) methyl,
(f) ethyl,
(g) hydroxyl,
(h) cyano,
(i) oxo,
(j) amino, and
(k) C₁₋₃alkylamino.

### DETAILED DESCRIPTION OF THE INVENTION

In one embodiment, the present application discloses a compound, or pharmaceutically acceptable salt thereof, wherein L¹ is selected from the group consisting of —C(O)—, —C(O)-heterocyclyl-, —C(O)-heterocyclyl-O—, —C(O)-heterocyclyl-N(R⁴)—, —C(O)-heterocyclyl-N(R⁴)C(O)—, —C(O)N(R⁴)—, —C(O)N(R⁴)—C₁₋₆alkylenyl-, —C(O)N(R⁴)—C₁₋₆alkylenyl-O—, —C(O)N(R⁴)—C₁₋₆alkylenyl-C₃₋₈cycloalkyl-, —C(O)N(R⁴)—C₁₋₆alkylenyl-C(O)—N(R⁴)—, —C(O)N(R⁴)-heterocyclyl-, —C(O)N(R⁴)-heterocyclyl-C(O)—, and 1,3,4-oxadiazolyl, wherein said heterocyclyl or cycloalkyl groups are unsubstituted or substituted with 1-3 R⁶ groups, and wherein heterocyclyl is a 4-7-membered ring containing 1-2 N heteroatoms.

In one class of this embodiment, L¹ is —C(O)-heterocyclyl-, wherein said heterocyclyl group is unsubstituted or substituted with 1-3 R⁶ groups, and wherein heterocyclyl is a 4-7-membered ring containing 1-2 N heteroatoms.

In another class of this embodiment, L¹ is —C(O)-heterocyclyl-O—, wherein said heterocyclyl group is unsubstituted or substituted with 1-3 R⁶ groups, and wherein heterocyclyl is a 4-7-membered ring containing 1-2 N heteroatoms.

In another class of this embodiment, L¹ is —C(O)-heterocyclyl-N(R⁴)—, wherein said heterocyclyl group is unsubstituted or substituted with 1-3 R⁶ groups, and wherein heterocyclyl is a 4-7-membered ring containing 1-2 N heteroatoms.

In another class of this embodiment, L¹ is —C(O)-heterocyclyl-N(R⁴)C(O)—, wherein said heterocyclyl group is unsubstituted or substituted with 1-3 R⁶ groups, and wherein heterocyclyl is a 4-7-membered ring containing 1-2 N heteroatoms.

In another class of this embodiment, L¹ is —C(O)N(R⁴)—. In another class of this embodiment, L¹ is —C(O)N(R⁴)—C₁₋₆alkylenyl-.

In another embodiment, L¹ is —C(O)N(R⁴)—C₁₋₆alkylenyl-O—.

In another class of this embodiment, L¹ is —C(O)N(R⁴)—C₁₋₆alkylenyl-C₃₋₈cycloalkyl-, wherein said cycloalkyl group is unsubstituted or substituted with 1-3 R⁶ groups.

In another class of this embodiment, L¹ is —C(O)N(R⁴)—C₁₋₆alkylenyl-C(O)—N(R⁴)—.

In another class of this embodiment L¹ is —C(O)N(R⁴)—C₁₋₆alkylenyl-C(O)—N(R⁴)—.

In another class of this embodiment, L¹ is —C(O)N(R⁴)-heterocyclyl-, wherein said heterocyclyl group is unsubstituted or substituted with 1-3 R⁶ groups, and wherein heterocyclyl is a 4-7-membered ring containing 1-2 N heteroatoms.

In another class of this embodiment, L¹ is —C(O)N(R⁴)-heterocyclyl-C(O)—, wherein said heterocyclyl group is unsubstituted or substituted with 1-3 R⁶ groups, and wherein heterocyclyl is a 4-7-membered ring containing 1-2 N heteroatoms.

In another class of this embodiment, L¹ is 1,3,4-oxadiazolyl.

In another class of this embodiment, L¹ is selected from the group consisting of:

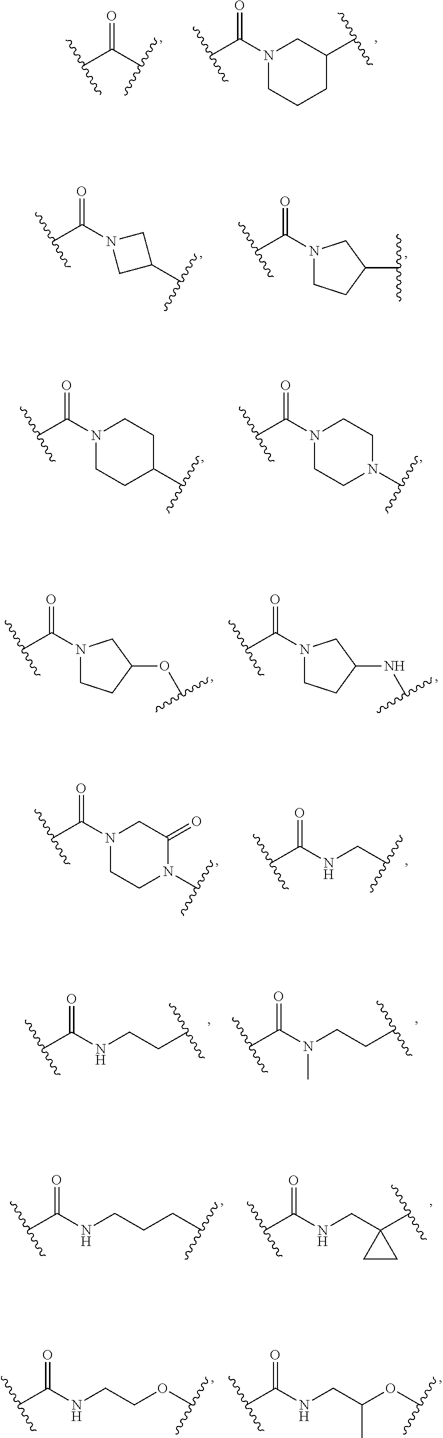

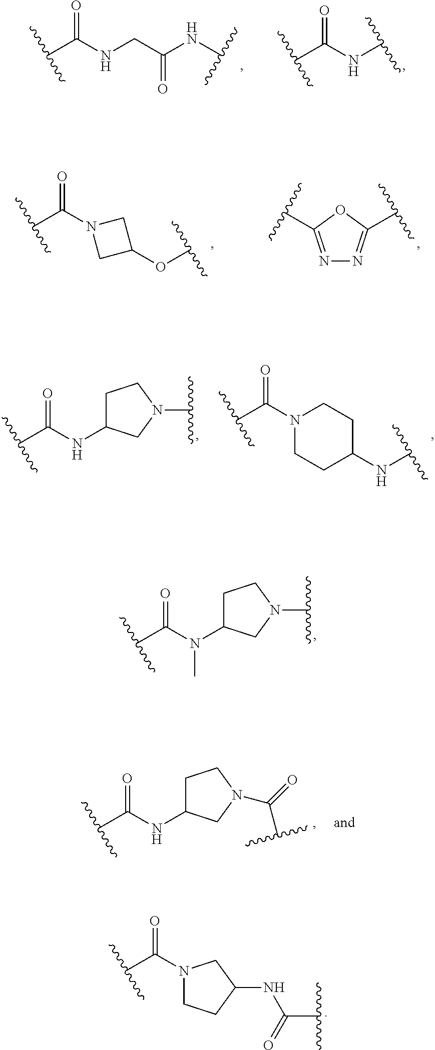

In another class of this embodiment, L¹ is selected from the group consisting of:

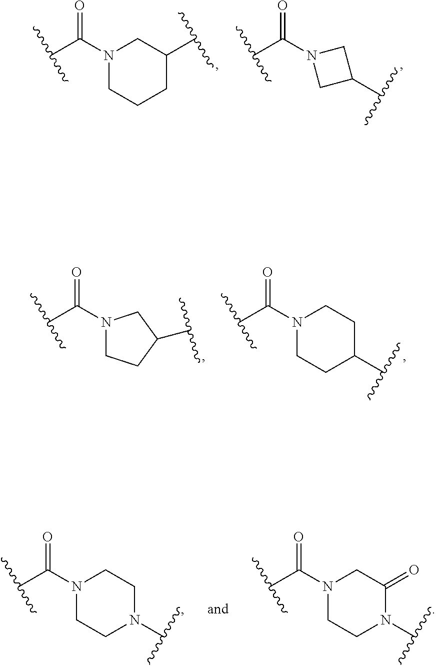 [SMILES: *C(=O)N1CCCC(*)C1 ; *C(=O)N1CC(*)C1 ; *C(=O)N1CCC(*)C1 ; *C(=O)N1CCC(*)CC1 ; *C(=O)N1CCN(*)CC1 ; *C(=O)N1CCN(*)C(=O)C1]

In another class of this embodiment, L¹ is selected from the group consisting of:

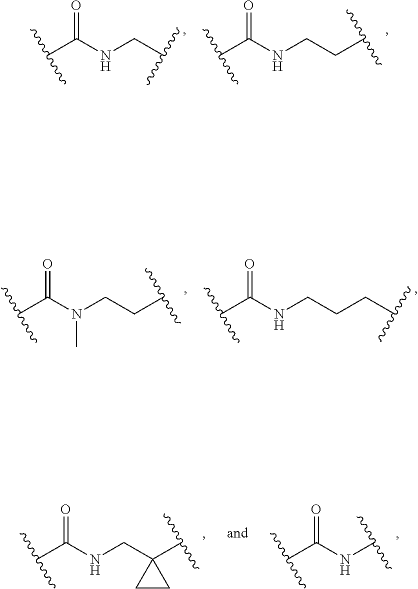

In another class of this embodiment, L¹ is

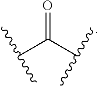 [SMILES: *C(*)=O]

In another class of this embodiment, L¹ is

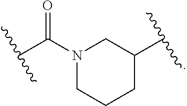 [SMILES: *C(=O)N1CCCC(*)C1]

In another class of this embodiment, L¹ is

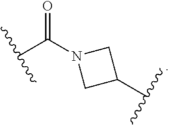 [SMILES: *C(=O)N1CC(*)C1]

In another class of this embodiment, L¹ is

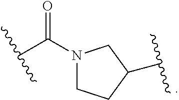 [SMILES: *C(=O)N1CCC(*)C1]

In another class of this embodiment, L¹ is

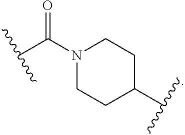 [SMILES: *C(=O)N1CCC(*)CC1]

In another class of this embodiment, L1 is

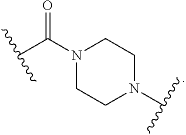 [SMILES: *C(=O)N1CCN(*)CC1]

In another class of this embodiment, L¹ is

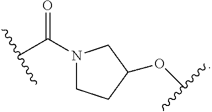 [SMILES: *OC1CCN(C(*)=O)C1]

In another class of this embodiment, L¹ is

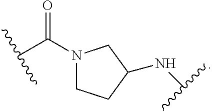 [SMILES: *NC1CCN(C(*)=O)C1]

In another class of this embodiment, L¹ is

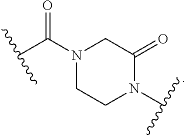 [SMILES: *C(=O)N1CCN(*)C(=O)C1]

In another class of this embodiment, L¹ is

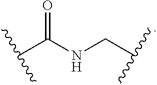 [SMILES: *CNC(*)=O]

In another class of this embodiment, L¹ is

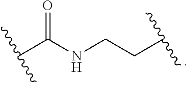 [SMILES: *CCNC(*)=O]

In another class of this embodiment, L¹ is

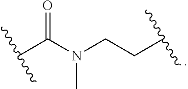 [SMILES: *CCN(C)C(*)=O]

In another class of this embodiment, L¹ is

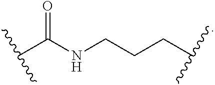 [SMILES: *CCCNC(*)=O]

In another class of this embodiment, L¹ is

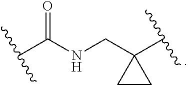 [SMILES: *C(=O)NCC1(*)CC1]

In another class of this embodiment, L¹ is

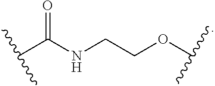 [SMILES: *OCCNC(*)=O]

In another class of this embodiment, L¹ is

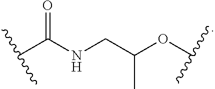 [SMILES: *OC(C)CNC(*)=O]

In another class of this embodiment, L¹ is

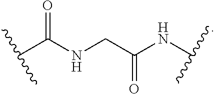 [SMILES: *NC(=O)CNC(*)=O]

In another class of this embodiment, L¹ is

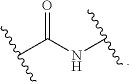 [SMILES: *NC(*)=O]

In another class of this embodiment, L¹ is

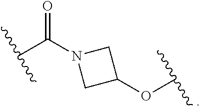 [SMILES: *OC1CN(C(*)=O)C1]

In another class of this embodiment, L¹ is

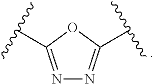 [SMILES: *c1nnc(*)o1]

In another class of this embodiment, L¹ is

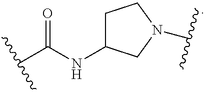 [SMILES: *C(=O)NC1CCN(*)C1]

In another class of this embodiment, L¹ is

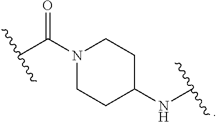 [SMILES: *NC1CCN(C(*)=O)CC1]

In another class of this embodiment, L¹ is

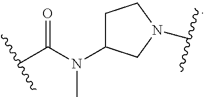 [SMILES: *C(=O)N(C)C1CCN(*)C1]

In another class of this embodiment, L¹ is

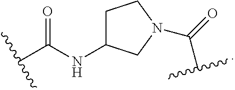 [SMILES: *C(=O)NC1CCN(C(*)=O)C1]

In another class of this embodiment, L¹ is

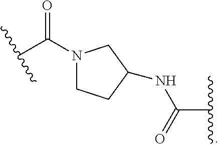 [SMILES: *C(=O)NC1CCN(C(*)=O)C1]

In another embodiment, the present application discloses a compound, or pharmaceutically acceptable salt thereof, wherein R¹ is selected from the group consisting of C₁₋₆alkyl, C₃₋₈cycloalkyl, C₃₋₈cycloalkenyl, 5 or 6-membered heterocyclyl, and 7- or 8-membered bicycloalkyl, wherein said alkyl, cycloalkyl, cylcoalkenyl, heterocyclyl, and bicycloalkyl groups are unsubstituted or substituted with 1-3 R² groups, and wherein said heterocyclyl, or heteroaryl rings have 1-4 heteroatoms selected from the group consisting of O, N, and S.

In one class of this embodiment, R¹ is C₁₋₆alkyl, wherein said alkyl group is unsubstituted or substituted with 1-3 R² groups. In another class of this embodiment, R¹ is C₃₋₈cycloalkyl-, wherein said cycloalkyl group is unsubstituted or substituted with 1-3 R² groups, wherein when said cycloalkyl has substituents on carbon atoms, said substituents can optionally be taken together with the carbon atoms to which they are attached to form a first three-, four-, five- or six-membered cycloalkyl, cycloalkenyl, heterocyclyl, heterocyclenyl, aryl or heteroaryl.

In another class of this embodiment, the present application discloses a compound, or pharmaceutically acceptable salt thereof, wherein R¹ is C₃₋₈cycloalkenyl-, wherein said cycloalkenyl group is unsubstituted or substituted with 1-3 R² groups. In another class of this embodiment, R¹ is 5 or 6-membered heterocyclyl, wherein said heterocyclyl group is unsubstituted or substituted with 1-3 R² groups, and wherein said heterocyclyl has 1-4 hetero-atoms selected from the group consisting of O, N, and S. In another class of this embodiment, R¹ is 7- or 8-membered bicycloalkyl, wherein said 7- or 8-membered bicycloalkyl group is unsubstituted or substituted with 1-3 R² groups.

In another class of this embodiment, R¹ is selected from the group consisting of the following:

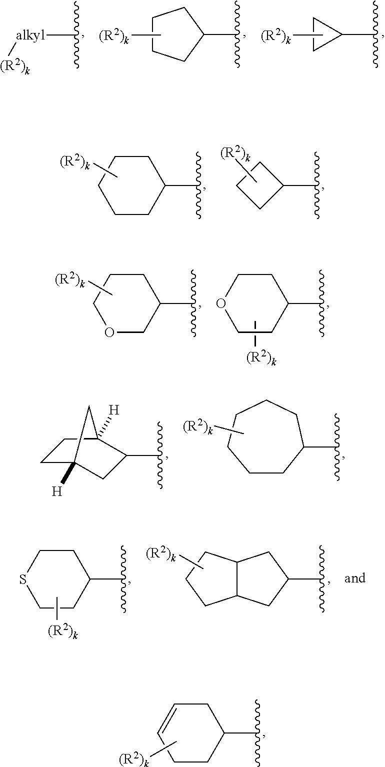

wherein k is 0, 1, 2 or 3.

In another class of this embodiment, R¹ is selected from the group consisting of the following:

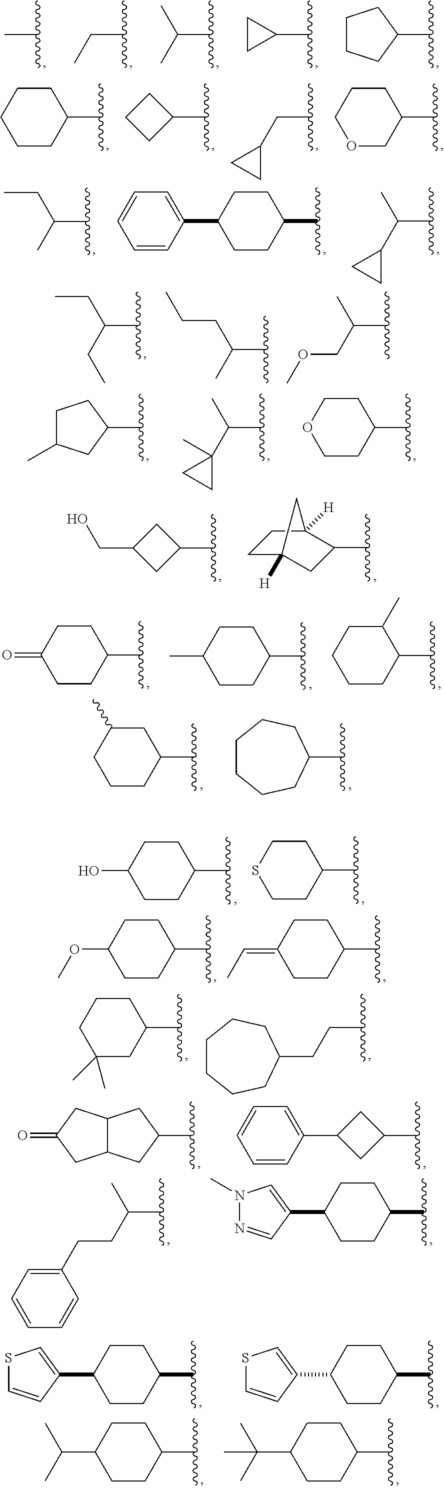

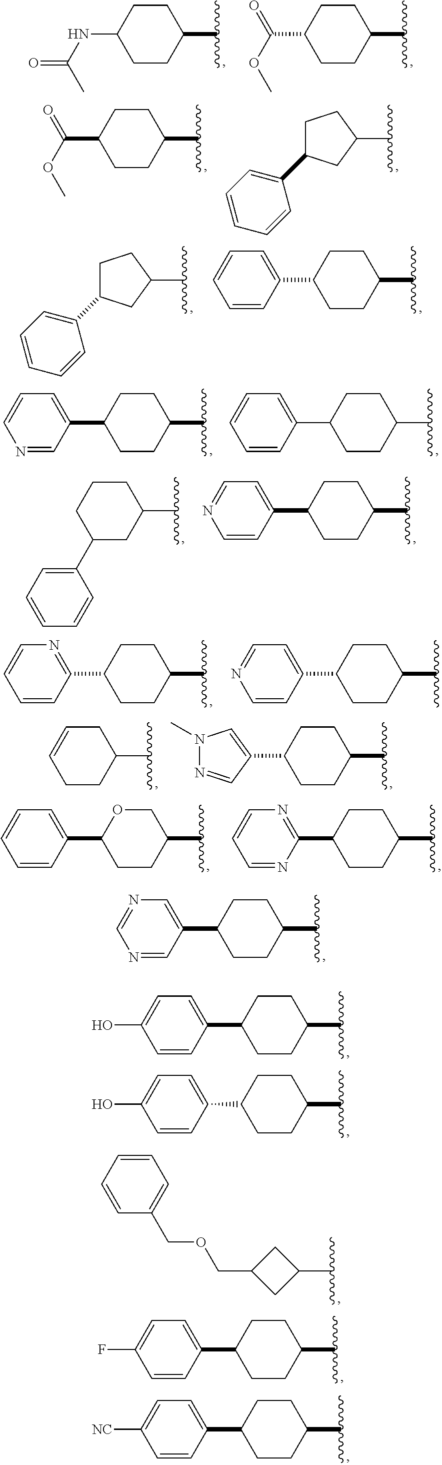

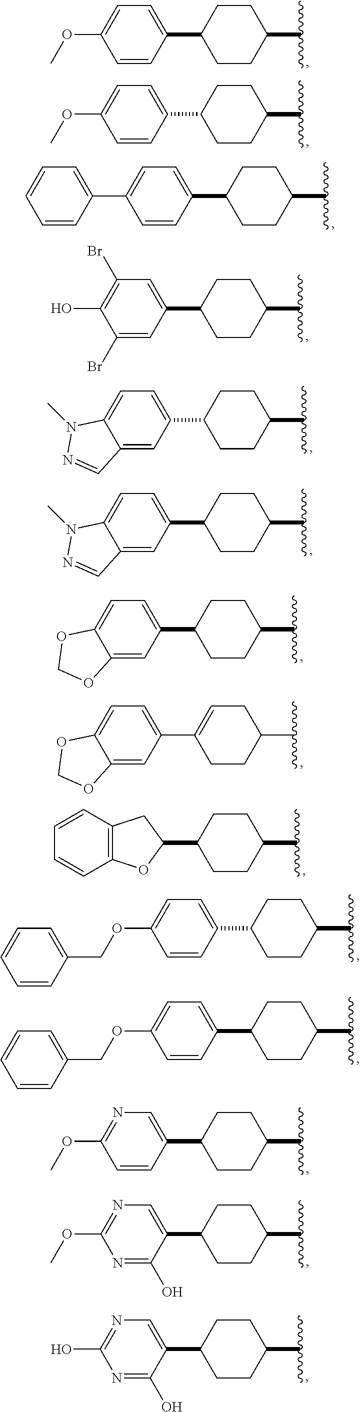

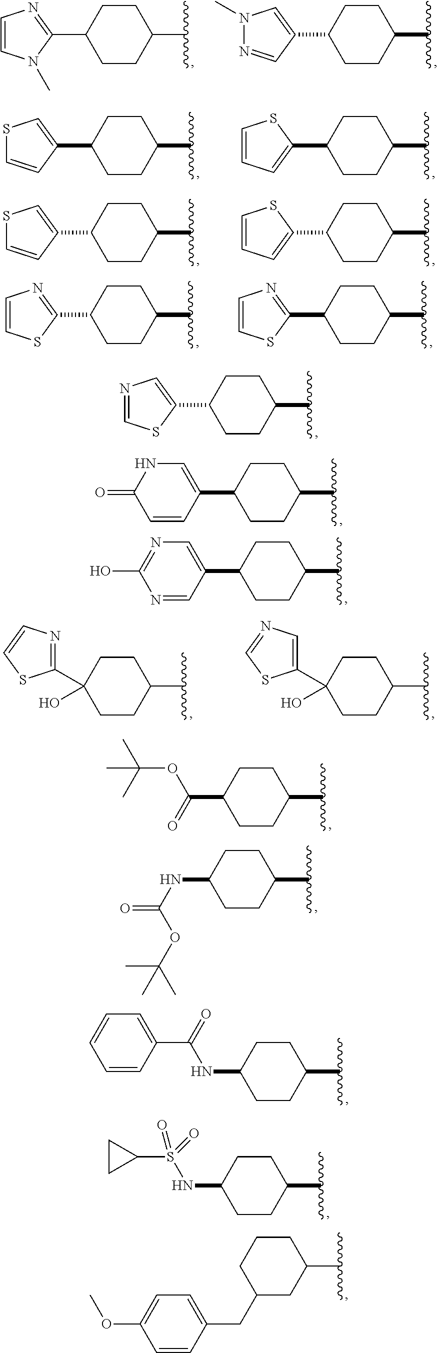

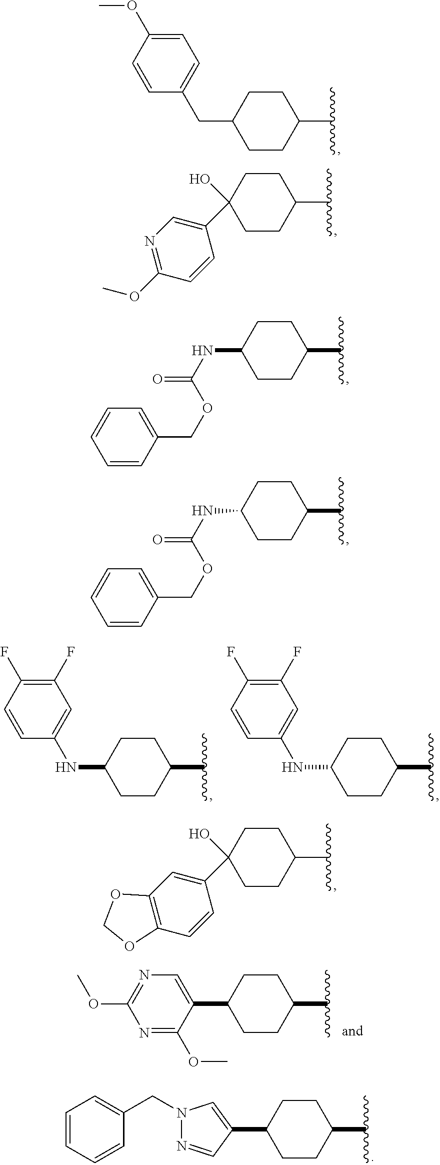

In another class of this embodiment, R¹ is

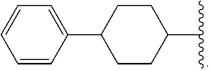 [SMILES: *C1CCC(c2ccccc2)CC1]

In another embodiment, the present application discloses a compound, or pharmaceutically acceptable salt thereof, wherein Ring A is selected from the group consisting of:
(a) aryl,
(b) C₃₋₈cycloalkyl,
(c) C₃₋₈cycloalkenyl,
(d) 5 or 6-membered heterocyclyl,
(e) 5 or 6-membered heterocyclenyl,
(f) 5 or 6-membered monocyclic heteroaryl,
(g) 9- or 10-membered cycloalkylaryl,
(h) 9- or 10-membered heterocyclylheteroaryl,
(i) 9- or 10-membered heterocyclylaryl, and
(j) 9- or 10-membered heteroarylaryl,

wherein said heterocyclyl, heterocyclenyl, or heteroaryl rings have 1-4 heteroatoms selected from the group consisting of O, N, and S, and wherein said aryl, cycloalkyl, cycloalkenyl, heterocyclyl, heterocyclenyl, heteroaryl, 9- or 10-membered cycloalkylaryl, 9- or 10-membered heterocyclylheteroaryl, 9- or 10-membered heterocyclylaryl, or 9- or 10-membered heteroarylaryl are unsubstituted or substituted with 1-3 R⁵ groups.

In one embodiment of this class, Ring A is aryl, unsubstituted or substituted with 1-3 R⁵ groups. In another class of this embodiment, Ring A is C₃₋₈cycloalkyl, unsubstituted or substituted with 1-3 R⁵ groups. In another class of this embodiment, Ring A is C₃₋₈cycloalkenyl, unsubstituted or substituted with 1-3 R⁵ groups.

In another class of this embodiment, Ring A is 5 or 6-membered heterocyclyl, wherein said heterocyclyl has 1-4 heteroatoms selected from the group consisting of O, N, and S, and wherein said heterocyclyl is unsubstituted or substituted with 1-3 R⁵ groups. In another class of this embodiment, Ring A is 5 or 6-membered heterocyclenyl, wherein said heterocyclenyl has 1-4 heteroatoms selected from the group consisting of O, N, and S, and wherein said heterocyclenyl is unsubstituted or substituted with 1-3 R⁵ groups.

In another class of this embodiment, Ring A is 5 or 6-membered monocyclic heteroaryl, wherein said heteroaryl ring has 1-4 heteroatoms selected from the group consisting of O, N, and S, and wherein said heteroaryl is unsubstituted or substituted with 1-3 R⁵ groups. In another class of this embodiment, Ring A is 9- or 10-membered cycloalkylaryl, wherein said 9- or 10-membered cycloalkylaryl is unsubstituted or substituted with 1-3 R⁵ groups, and wherein said 9- or 10-membered cycloalkylaryl is unsubstituted or substituted with 1-3 R⁵ groups.

In another class of this embodiment, Ring A is 9- or 10-membered heterocyclylheteroaryl, wherein said 9- or 10-membered heterocyclylheteroaryl has 1-4 hetero-atoms selected from the group consisting of O, N, and S, and wherein said 9- or 10-membered heterocyclylheteroaryl is unsubstituted or substituted with 1-3 R⁵ groups. In another class of this embodiment, Ring A is 9- or 10-membered heterocyclylaryl, wherein said 9- or 10-membered heterocyclylaryl has 1-4 hetero-atoms selected from the group consisting of O, N, and S, and wherein said 9- or 10-membered heterocyclylheteroaryl is unsubstituted or substituted with 1-3 R⁵ groups.

In another class of this embodiment, Ring A is 9- or 10-membered heteroarylaryl, wherein said 9- or 10-membered heteroarylaryl has 1-4 hetero-atoms selected from the group consisting of O, N, and S, and wherein said 9- or 10-membered heteroarylaryl is unsubstituted or substituted with 1-3 R⁵ groups.

In one class of this embodiment, Ring A is selected from the group consisting of:

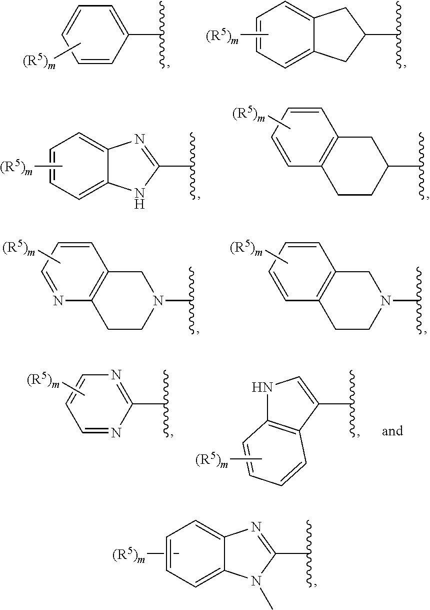

wherein m is 0, 1, 2 or 3.

In another class of this embodiment, Ring A is selected from the group consisting of:

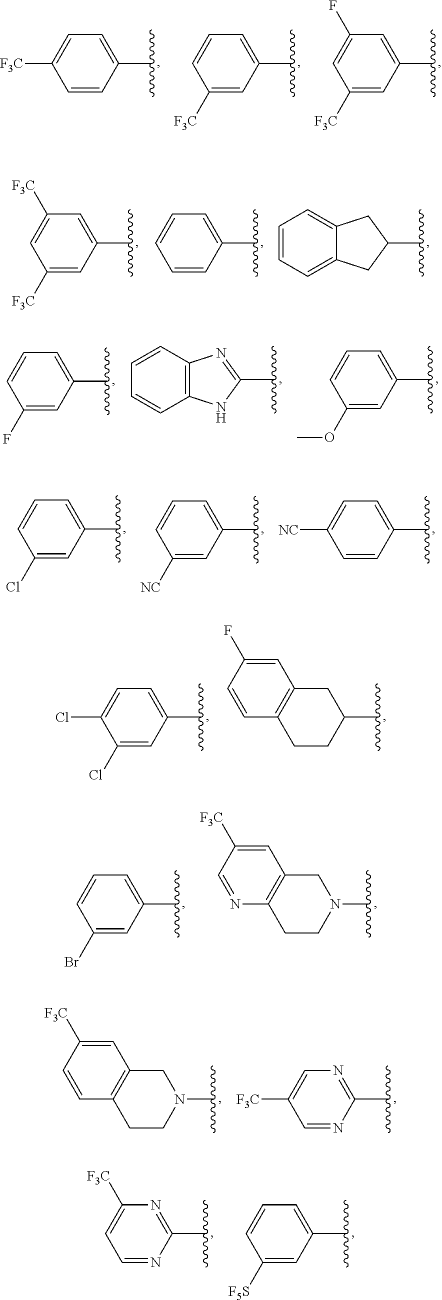

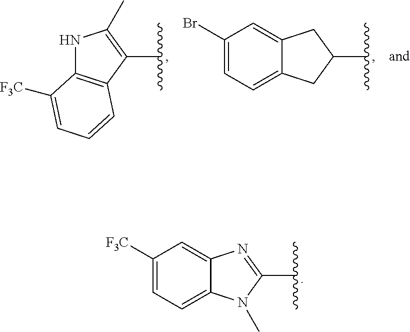 [SMILES: *c1c(C)[nH]c2c(C(F)(F)F)cccc12 ; *C1Cc2ccc(Br)cc2C1 ; *c1nc2cc(C(F)(F)F)ccc2n1C]

In another class of this embodiment, Ring A is

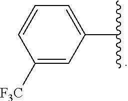 [SMILES: *c1cccc(C(F)(F)F)c1]

In another class of this embodiment, Ring A is

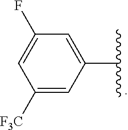 [SMILES: *c1cc(F)cc(C(F)(F)F)c1]

In another class of this embodiment, Ring A is

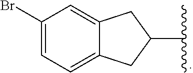 [SMILES: *C1Cc2ccc(Br)cc2C1]

In another class of this embodiment, Ring A is

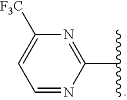 [SMILES: *c1nccc(C(F)(F)F)n1]

In another embodiment, the present application discloses a compound, or pharmaceutically acceptable salt thereof, wherein each R² is independently selected from the group consisting of hydrogen, HO—, halo, C₁₋₆alkyl, —C₁₋₆alkyl-OH, —O—C₁₋₆alkyl, aryl-C₁₋₆alkyl-O—C₁₋₆alkyl-, C₁₋₆alkenyl, oxo, —NH—C(O)—C₁₋₆alkyl, —CO₂—C₁₋₆alkyl, —NH—CO₂—C₁₋₆alkyl, —NH—C(O)-aryl, —NH—SO₂—C₃₋₈cycloalkyl, —NH-aryl, aryl, aryl-C₁₋₆alkyl-, C₃₋₈cycloalkyl, 5-7-membered heterocyclyl, 5-7-membered heterocyclenyl, 5 or 6-membered heteroaryl, and 9-membered heteroarylaryl, wherein said alkyl, aryl, cycloalkyl, heterocyclyl, heterocyclenyl and heteroaryl groups are unsubstituted or substituted with 1-3 R³ groups, and wherein said heterocyclyl, heterocyclenyl, or heteroaryl rings have 1-4 heteroatoms selected from the group consisting of O, N and S.

In one class of this embodiment, each R² is independently selected from the group consisting of C₃₋₈cycloalkyl, aryl, 5-7-membered heterocyclyl, 5-7-membered heterocyclenyl, 5 or 6-membered heteroaryl, and 9-membered heteroarylaryl, wherein said cycloalkyl, aryl, heterocyclyl, heterocyclenyl and heteroaryl groups are each unsubstituted or substituted with 1-3 R³ groups, and wherein said heterocyclyl, heterocyclenyl, or heteroaryl rings have 1-4 hetero-atoms selected from the group consisting of O, N and S.

In another class of this embodiment, each R² is independently aryl, wherein said aryl group is unsubstituted or substituted with 1-3 R³ groups. In another class of this embodiment, each R² is independently 5-7-membered heterocyclyl, wherein said heterocyclyl group is unsubstituted or substituted with 1-3 R³ groups, and wherein said heterocyclyl has 1-4 hetero-atoms selected from the group consisting of O, N and S.

In another class of this embodiment, each R² is independently 5 or 6-membered heteroaryl, wherein said heteroaryl group is unsubstituted or substituted with 1-3 R³ groups, and wherein said heteroaryl has 1-4 heteroatoms selected from the group consisting of O, N and S. In another class of this embodiment, each R² is independently 9-membered heteroarylaryl, wherein said 9-membered heteroarylaryl group is unsubstituted or substituted with 1-3 R³ groups, and wherein said heteroaryl has 1-4 heteroatoms selected from the group consisting of O, N and S.

In another class of this embodiment, each R² is independently selected from the group consisting of:

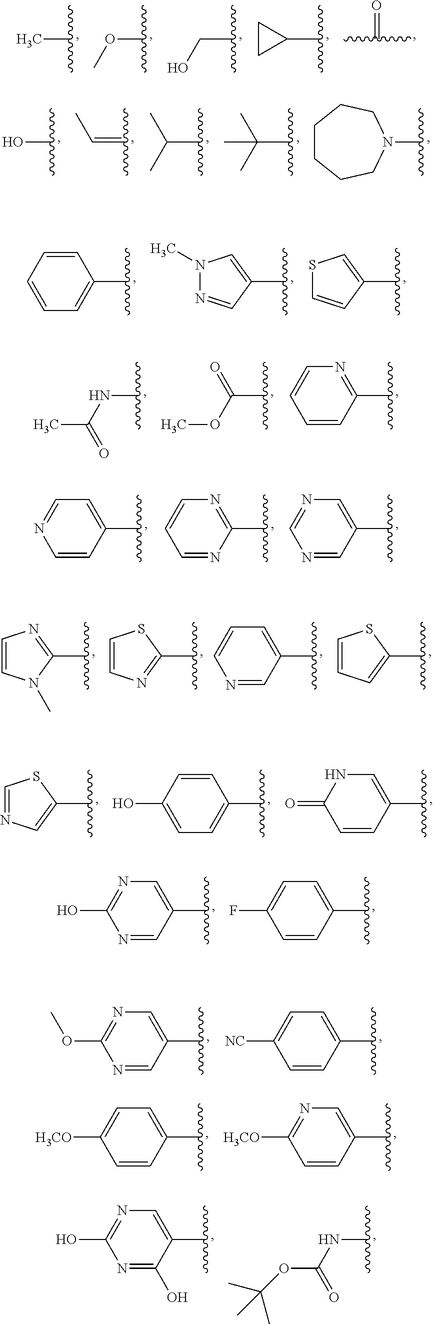

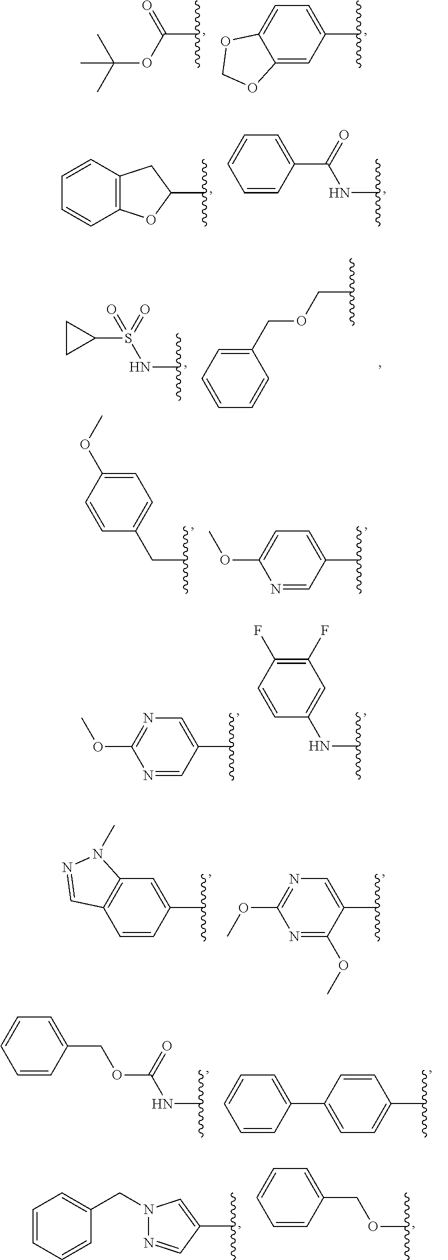

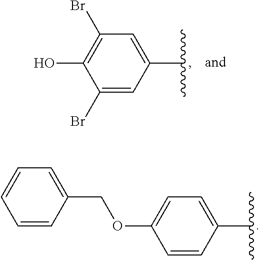 [SMILES: *c1cc(Br)c(O)c(Br)c1 ; *c1ccc(OCc2ccccc2)cc1]

In one embodiment, the present application discloses a compound, or pharmaceutically acceptable salt thereof, wherein L¹ is selected from the group consisting of:

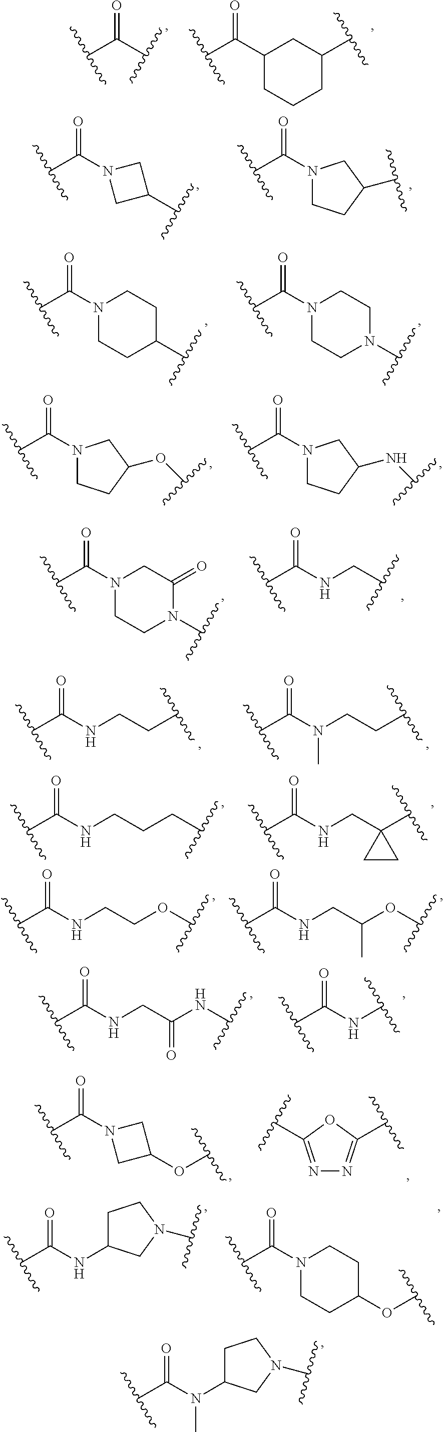

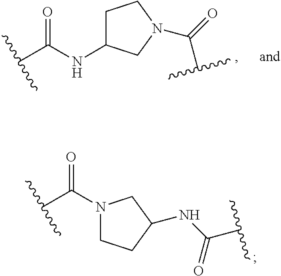 [SMILES: *C(=O)NC1CCN(C(*)=O)C1 ; *C(=O)NC1CCN(C(*)=O)C1]

R¹ is selected from the group consisting of the following:

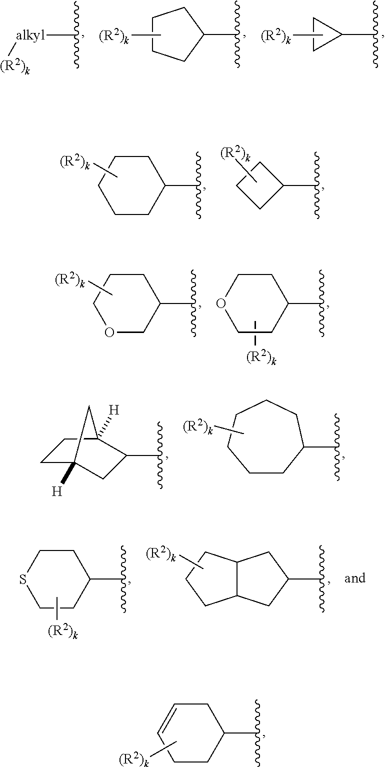

wherein k is 0, 1, 2 or 3; and

Ring A is selected from the group consisting of:

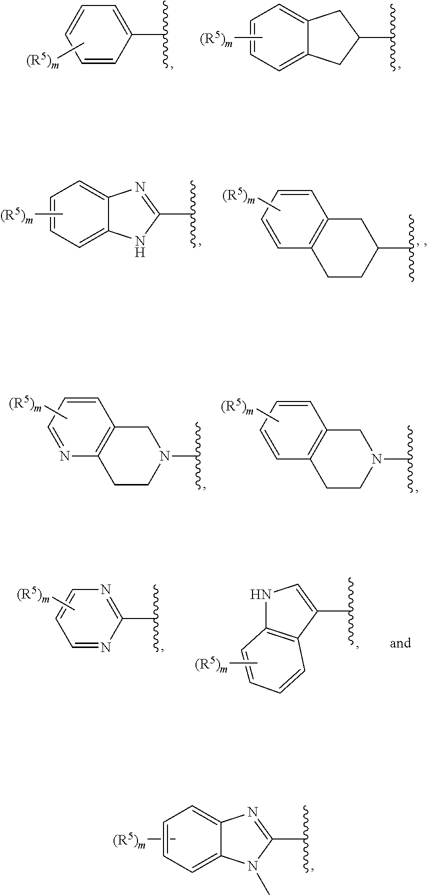

wherein m is 0, 1, 2, or 3. In a class of this embodiment, L¹ is

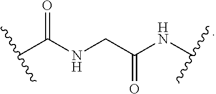 [SMILES: *NC(=O)CNC(*)=O]

In one class of this embodiment, R¹ is selected from the group consisting of the following:

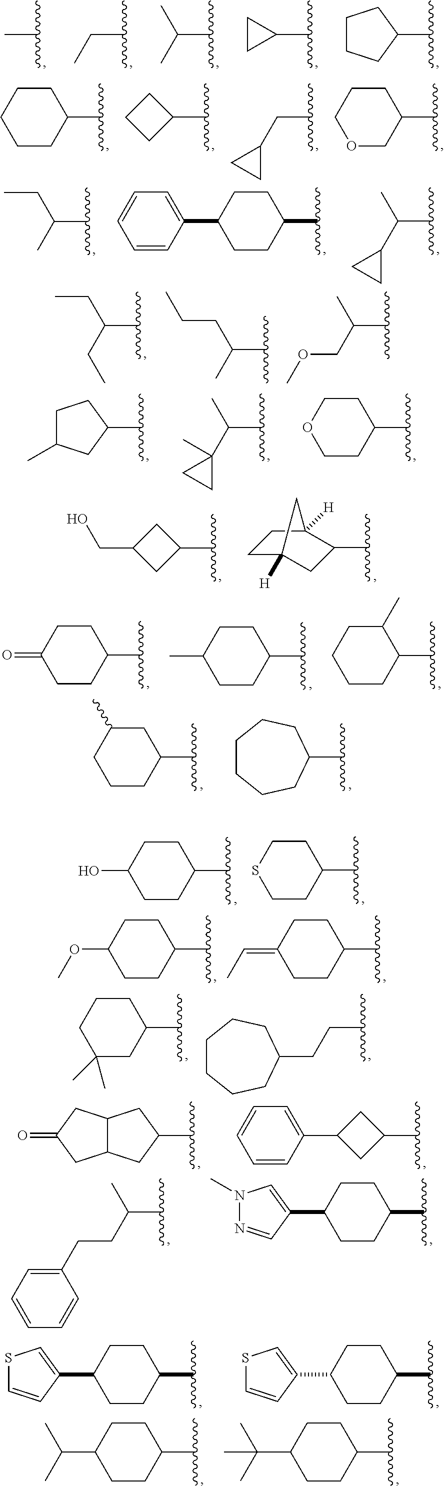

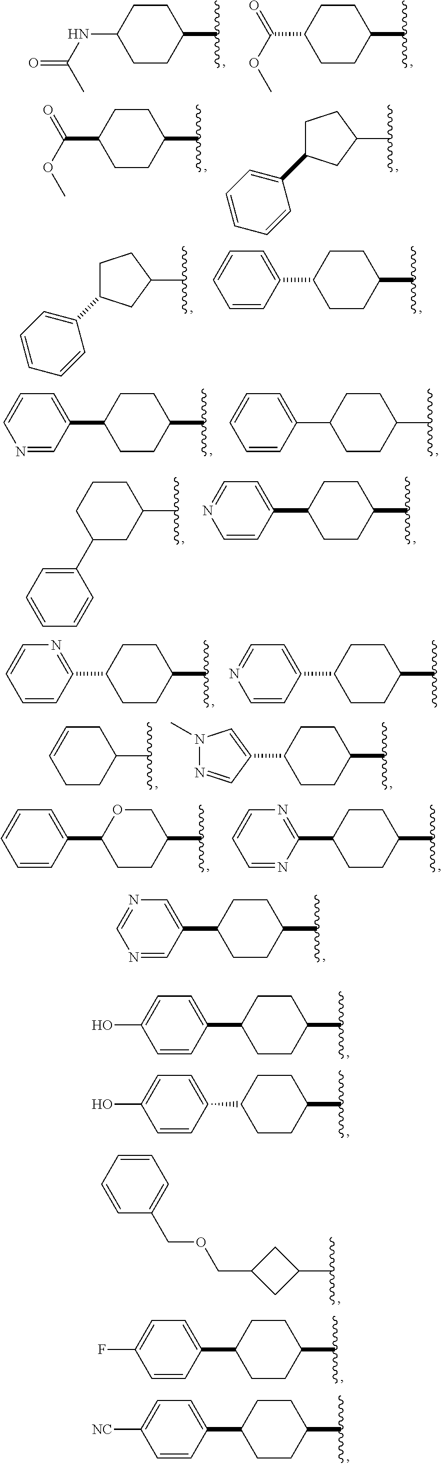

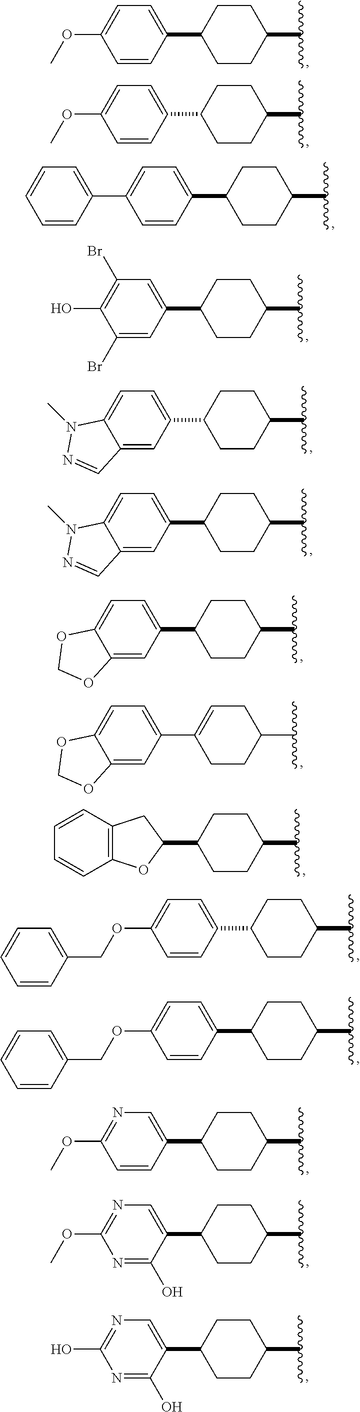

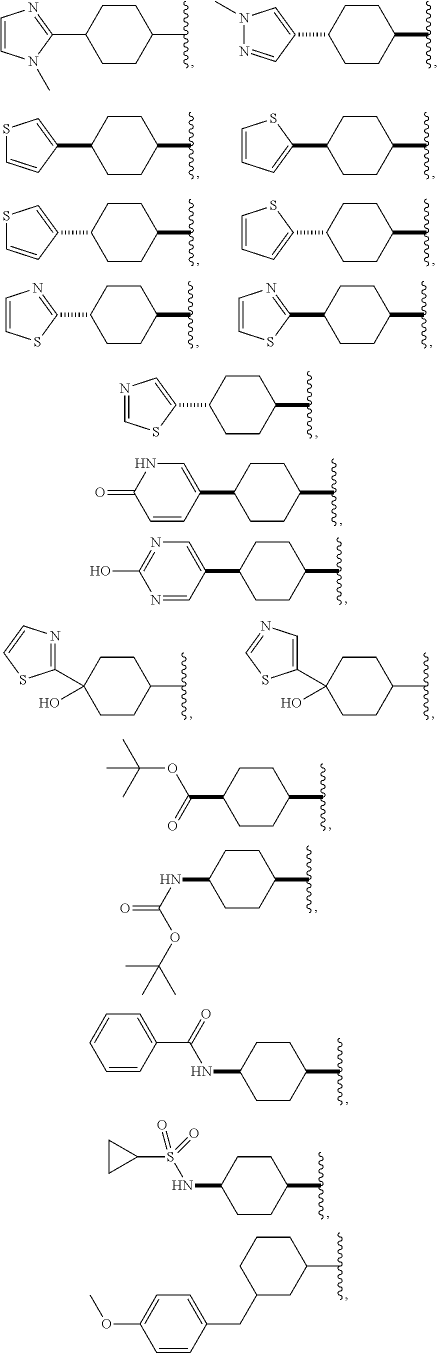

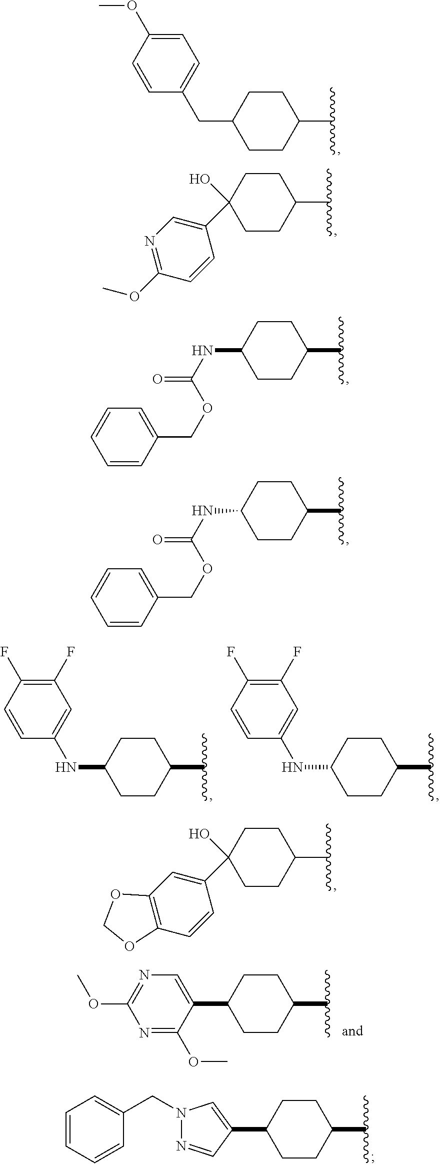

and

Ring A is selected from the group consisting of:

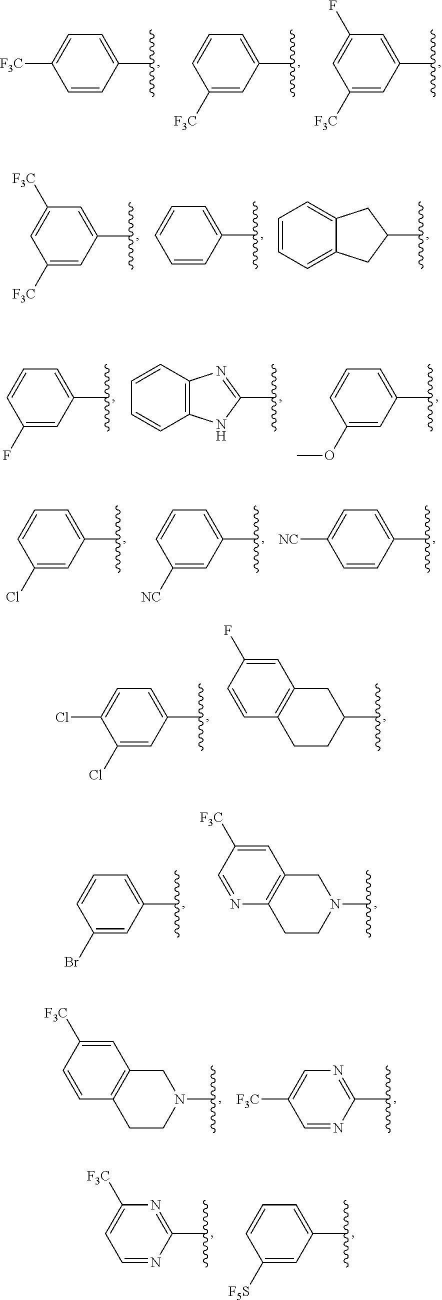

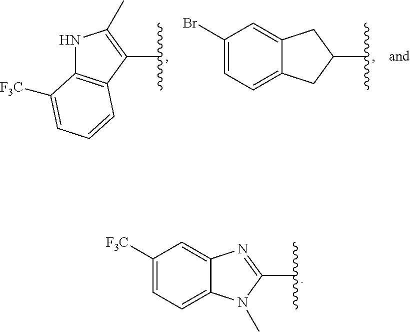 [SMILES: *c1c(C)[nH]c2c(C(F)(F)F)cccc12 ; *C1Cc2ccc(Br)cc2C1 ; *c1nc2cc(C(F)(F)F)ccc2n1C]

In a subclass of this class, L¹ is

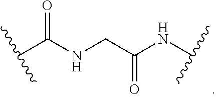 [SMILES: *NC(=O)CNC(*)=O]

In one embodiment, the present application discloses a compound, or pharmaceutically acceptable salt thereof, wherein L¹ is 1,3,4-oxadiazolyl;

R¹ is

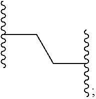 [SMILES: *CC*]

and

Ring A is selected from the group consisting of:

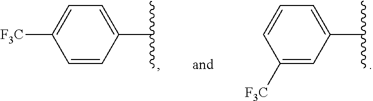 [SMILES: *c1ccc(C(F)(F)F)cc1 ; *c1cccc(C(F)(F)F)c1]

In one embodiment, the present application discloses a compound, or pharmaceutically acceptable salt thereof, wherein L¹ is

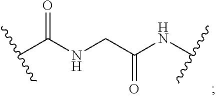 [SMILES: *NC(=O)CNC(*)=O]

and

Ring A is aryl.

In one embodiment, the present application discloses a compound, or pharmaceutically acceptable salt thereof, wherein L¹ is

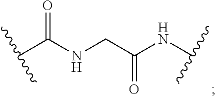 [SMILES: *NC(=O)CNC(*)=O]

Ring A is aryl; and

R¹ is C₃₋₈cycloalkyl-, wherein said cycloalkyl group is unsubstituted or substituted with 1-3 R² groups.

In one embodiment, L¹ is

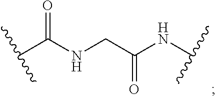 [SMILES: *NC(=O)CNC(*)=O]

and

Ring A is

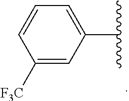 [SMILES: *c1cccc(C(F)(F)F)c1]

In one embodiment, the compounds of the present invention include those of Formula II:

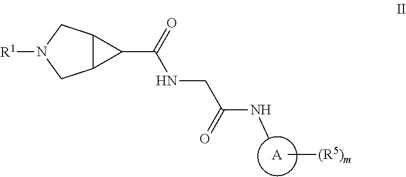

wherein Ring A, R¹, R⁵, and m are defined herein, or a pharmaceutically acceptable salt thereof.

In one embodiment, the compounds of the present invention include those of Formula IIa:

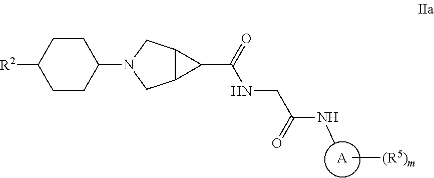

wherein Ring A, R², R⁵, and m are defined herein, or a pharmaceutically acceptable salt thereof.

In one embodiment, the compounds of the present invention include those of Formula IIb:

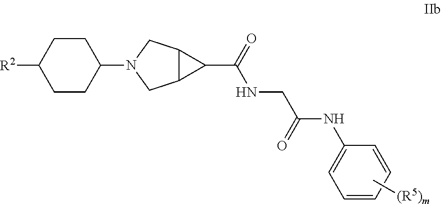

wherein R², R⁵ and m are defined herein, or a pharmaceutically acceptable salt thereof.

In one embodiment, the compounds of the present invention include those of Formula IIc:

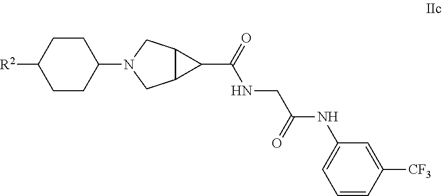

wherein R² is defined herein, or a pharmaceutically acceptable salt thereof.

In one embodiment, the compounds of the present invention include those of Formula III:

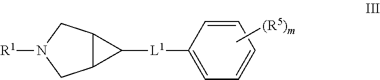

wherein L¹, R¹, R⁵, and m are defined herein, or a pharmaceutically acceptable salt, ester, solvate or prodrug thereof.

In one embodiment, the compounds of the present invention include those of Formula IV:

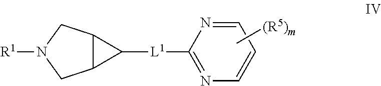

wherein L¹, R¹, R⁵, and m are defined herein, or a pharmaceutically acceptable salt, ester, solvate or prodrug thereof.

In one embodiment, the compounds of the present invention include those of Formula V:

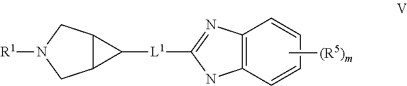

wherein L¹, R¹, R⁵, and m are defined herein, or a pharmaceutically acceptable salt, ester, solvate or prodrug thereof.

In one embodiment, the compounds of the present invention include those of Formula VI

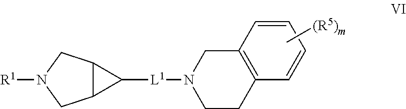

wherein L¹, R¹, R⁵, and m are defined herein, or a pharmaceutically acceptable salt, ester, solvate or prodrug thereof.

In one embodiment, the compounds of the present invention include those of Formula VI

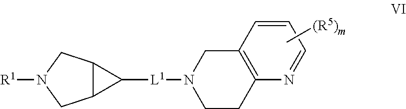

wherein L¹, R¹, R⁵, and m are defined herein, or a pharmaceutically acceptable salt, ester, solvate or prodrug thereof.

In one embodiment, the compounds of the present invention include those of Formula VIII

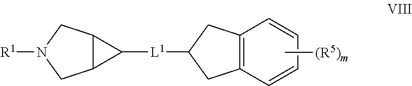

wherein L¹, R¹, R⁵, and m are defined herein, or a pharmaceutically acceptable salt, ester, solvate or prodrug thereof.

In one embodiment, the compounds of the present invention include those of Formula VIII

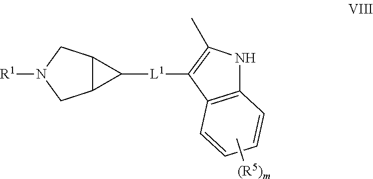

wherein L¹, R¹, R⁵, and m are defined herein, or a pharmaceutically acceptable salt, ester, solvate or prodrug thereof.

Representative compounds of the present invention include those presented in the Examples and pharmaceutically salts and individual stereoisomers thereof.

Non-limiting examples of compounds of the present invention include those disclosed in Table 1, or a pharmaceutically acceptable salt, solvate, hydrate, ester, prodrug or stereoisomer thereof.

The present invention provides pharmaceutical compositions comprising said compounds, or a pharmaceutically acceptable salt, solvate, hydrate, ester, prodrug or stereoisomer thereof.

In one embodiment, the present invention provides an isolated or purified form of a compound of Formula I, or a pharmaceutically acceptable salt, solvate or ester thereof.

In another embodiment, the present invention provides a compound of Formula I, at least 90% pure.

In another embodiment, the present invention provides a compound of Formula I, at least 95% pure.

In yet another embodiment, the present invention provides a compound of Formula I, at least 99% pure.

In one embodiment, the invention is directed to a pharmaceutical composition comprising an effective amount of at least one compound of Formula I, or a pharmaceutically acceptable salt or ester thereof, and a pharmaceutically acceptable carrier.

In one embodiment, the invention is directed to a pharmaceutical composition comprising an effective amount of at least one compound of Formula I, at least one other active pharmaceutically active ingredient, and a pharmaceutically acceptable carrier.

Mammalian chemokine receptors provide a target for interfering with or promoting eosinophil and/or lymphocyte function in a mammal, such as a human. Compounds which inhibit or promote chemokine receptor function, are particularly useful for modulating eosinophil and/or lymphocyte function for therapeutic purposes. Accordingly, compounds which inhibit or promote chemokine receptor function would be useful in treating, preventing, ameliorating, controlling or reducing the risk of a wide variety of inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis.

For example, an instant compound which inhibits one or more functions of a mammalian chemokine receptor (e.g., a human chemokine receptor) may be administered to inhibit (i.e., reduce or prevent) inflammation. As a result, one or more inflammatory processes, such as leukocyte emigration, chemotaxis, exocytosis (e.g., of enzymes, histamine) or inflammatory mediator release, is inhibited.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

Diseases and conditions associated with inflammation and infection can be treated using the compounds of the present invention. In one embodiment, the disease or condition is one in which the actions of lymphocytes are to be inhibited or promoted, in order to modulate the inflammatory response.

Diseases or conditions of humans or other species which can be treated with inhibitors of chemokine receptor function, include, but are not limited to: inflammatory or allergic diseases and conditions, including respiratory allergic diseases such as asthma, particularly bronchial asthma, allergic rhinitis, hypersensitivity lung diseases, COPD, hypersensitivity pneumonitis, eosinophilic pneumonias (e.g., Loeffler's syndrome, chronic eosinophilic pneumonia), delayed-type hypersensitivity, interstitial lung diseases (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies (e.g., to penicillin, cephalosporins), insect sting allergies; autoimmune diseases, such as rheumatoid arthritis, psoriatic arthritis, multiple sclerosis, systemic lupus erythematosus, myasthenia gravis, juvenile onset diabetes; glomerulonephritis, autoimmune thyroiditis, Behcet's disease; graft rejection (e.g., in transplantation), including allograft rejection or graft-versus-host disease; inflammatory bowel diseases, such as Crohn's disease and ulcerative colitis; spondyloarthropathies; scleroderma; psoriasis (including T-cell mediated psoriasis) and inflammatory dermatoses such an dermatitis, eczema, atopic dermatitis, allergic contact dermatitis, urticaria; vasculitis (e.g., necrotizing, cutaneous, and hypersensitivity vasculitis); eosinophilic myositis, eosinophilic fasciitis; cancers with leukocyte infiltration of the skin or organs, stroke, Other diseases or conditions in which undesirable inflammatory responses are to be inhibited can be treated, including, but not limited to, reperfusion injury, atherosclerosis, certain hematologic malignancies, cytokine-induced toxicity (e.g., septic shock, endotoxic shock), polymyositis, dermatomyositis.

Diseases or conditions of humans or other species which can be treated with modulators of chemokine receptor function, include, but are not limited to: immunosuppression, such as that in individuals with immunodeficiency syndromes such as AIDS or other viral infections, individuals undergoing radiation therapy, chemotherapy, therapy for autoimmune disease or drug therapy (e.g., corticosteroid therapy), which causes immunosuppression; immunosuppression due to congenital deficiency in receptor function or other causes; and infections diseases, such as parasitic diseases, including, but not limited to helminth infections, such as nematodes (round worms), (Trichuriasis, Enterobiasis, Ascariasis, Hookworm, Strongyloidiasis, Trichinosis, filariasis), trematodes (flukes) (Schistosomiasis, Clonorchiasis), cestodes (tape worms) (Echinococcosis, Taeniasis saginata, Cysticercosis), visceral worms, visceral larva migraines (e.g., Toxocara), eosinophilic gastroenteritis (e.g., Anisaki sp., Phocanema sp.), and cutaneous larva migraines (Ancylostona braziliense, Ancylostoma caninum). In addition, treatment of the aforementioned inflammatory, allergic and autoimmune diseases can also be contemplated for promoters of chemokine receptor function if one contemplates the delivery of sufficient compound to cause the loss of receptor expression on cells through the induction of chemokine receptor internalization or delivery of compound in a manner that results in the misdirection of the migration of cells.

The compounds of the present invention are accordingly useful in treating, preventing, ameliorating, controlling or reducing the risk of a wide variety of inflammatory and immunoregulatory disorders and diseases, allergic conditions, atopic conditions, as well as autoimmune pathologies. In a specific embodiment, the present invention is directed to the use of the subject compounds for treating, preventing, ameliorating, controlling or reducing the risk of autoimmune diseases, such as rheumatoid arthritis or psoriatic arthritis.

The compounds of the present invention are accordingly useful for the treatment in a mammal of an inflammatory or immunoregulatory disorder or disease responsive to modulation of chemokine receptor function, including CCR2. In a specific embodiment, the present invention is directed to the use of the subject compounds for treating rheumatoid arthritis.

In another embodiment, the instant invention may be used to evaluate putative specific agonists or antagonists of chemokine receptors, including CCR2. Accordingly, the present invention is directed to the use of these compounds in the preparation and execution of screening assays for compounds that modulate the activity of chemokine receptors. For example, the compounds of this invention are useful for isolating receptor mutants, which are excellent screening tools for more potent compounds. Furthermore, the compounds of this invention are useful in establishing or determining the binding site of other compounds to chemokine receptors, e.g., by competitive inhibition. The compounds of the instant invention are also useful for the evaluation of putative specific modulators of the chemokine receptors, including CCR2. As appreciated in the art, thorough evaluation of specific agonists and antagonists of the above chemokine receptors has been hampered by the lack of availability of non-peptidyl (metabolically resistant) compounds with high binding affinity for these receptors. Thus the compounds of this invention are commercial products to be sold for these purposes.

The present invention is further directed to a method for the manufacture of a medicament for modulating chemokine receptor activity in humans and animals comprising combining a compound of the present invention with a pharmaceutical carrier or diluent.

The compounds of the present invention are useful for the manufacture of a medicament for use in treating an inflammatory or immunoregulatory disorder or disease responsive to modulation of chemokine receptor activity, including CCR2, in humans and animals comprising a compound of the present invention with a pharmaceutical carrier or diluent. In a specific embodiment, the inflammatory or immunoregulatory disorder or disease is rheumatoid arthritis.

The present invention is further directed to the use of the present compounds in treating, preventing, ameliorating, controlling or reducing the risk of infection by a retrovirus, in particular, herpes virus or the human immunodeficiency virus (HIV) and the treatment of, and delaying of the onset of consequent pathological conditions such as AIDS. Treating AIDS or preventing or treating infection by HIV is defined as including, but not limited to, treating a wide range of states of HIV infection: AIDS, ARC (AIDS related complex), both symptomatic and asymptomatic, and actual or potential exposure to HIV. For example, the compounds of this invention are useful in treating infection by HIV after suspected past exposure to HIV by, e.g., blood transfusion, organ transplant, exchange of body fluids, bites, accidental needle stick, or exposure to patient blood during surgery.

In an aspect of the present invention, a subject compound may be used in a method of inhibiting the binding of a chemokine to a chemokine receptor, such as CCR2, of a target cell, which comprises contacting the target cell with an amount of the compound which is effective at inhibiting the binding of the chemokine to the chemokine receptor.

Combined therapy to modulate chemokine receptor activity for thereby treating, preventing, ameliorating, controlling or reducing the risk of inflammatory and immunoregulatory disorders and diseases, including asthma and allergic diseases, as well as autoimmune pathologies such as rheumatoid arthritis and atherosclerosis, and those pathologies noted above is illustrated by the combination of the compounds of this invention and other compounds which are known for such utilities.

For example, in treating, preventing, ameliorating, controlling or reducing the risk of inflammation, the present compounds may be used in conjunction with an antiinflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, aspirin, codeine, embrel, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextramethorphan; a diuretic; and a sedating or non-sedating antihistamine.

Likewise, compounds of the present invention may be used in combination with other drugs that are used in the treatment/prevention/suppression or amelioration of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is typically employed. Accordingly, the pharmaceutical compositions of the present invention include those that also contain one or more other active ingredients, in addition to a compound of the present invention.

Examples of other active ingredients that may be combined with a compound of the present invention, either administered separately or in the same pharmaceutical compositions, include, but are not limited to: (a) VLA-4 antagonists such as those described in U.S. Pat. No. 5,510,332, WO95/15973, WO96/01644, WO96/06108, WO96/20216, WO96/22966, WO96/31206, WO96/40781, WO97/03094, WO97/02289, WO 98/42656, WO98/53814, WO98/53817, WO98/53818, WO98/54207, and WO98/58902; (b) steroids such as beclomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, desloratadine, cetirizine, fexofenadine, descarboethoxyloratadine, and the like; (e) non-steroidal anti-asthmatics such as β2-agonists (terbutaline, metaproterenol, fenoterol, isoetharine, albuterol, bitolterol, and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors; (h) inhibitors of phosphodiesterase type IV (PDE-IV); (i) other antagonists of the chemokine receptors, especially CCR-1, CCR2, CCR-3, CXCR-3 and CCR-5; (j) cholesterol lowering agents such as HMG-CoA reductase inhibitors (lovastatin, simvastatin and pravastatin, fluvastatin, atorvastatin, rosuvastatin, and other statins), sequestrants (cholestyramine and colestipol), cholesterol absorption inhibitors (ezetimibe), nicotinic acid, fenofibric acid derivatives (gemfibrozil, clofibrat, fenofibrate and benzafibrate), and probucol; (k) anti-diabetic agents such as insulin, sulfonylureas, biguanides (metformin), α-glucosidase inhibitors (acarbose) and glitazones (troglitazone and pioglitazone); (l) preparations of interferon beta (interferon beta-1α, interferon beta-1β), (m) other compounds such as 5-aminosalicylic acid and prodrugs thereof, antimetabolites such as azathioprine and 6-mercaptopurine, and cytotoxic cancer chemotherapeutic agents.

### DEFINITIONS

As used above, and throughout this disclosure, the following terms, unless otherwise indicated, shall be understood to have the following meanings:

“Patient” includes both human and animals.

“Mammal” means humans and other mammalian animals.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, oxime (e.g., ═N—OH), —NH(alkyl), —NH(cycloalkyl), —N(alkyl)₂, —O—C(O)-alkyl, —O—C(O)-aryl, —O—C(O)-cycloalkyl, —SF₅, carboxy and —C(O)O-alkyl. Non-limiting examples of suitable alkyl groups include methyl, ethyl, n-propyl, isopropyl and t-butyl.

“Haloalkyl” means an aliphatic hydrocarbon group which may be straight or branched and comprising about 1 to about 6 carbon atoms in the chain, which is substituted with 1 to 5 halogen groups. Non-limiting examples of suitable haloalkyl groups include chloromethyl, bromomethyl, fluoroethyl, dichloroethyl, and trifluoromethyl.

“Fluoroalkyl” means an aliphatic hydrocarbon group which may be straight or branched and comprising about 1 to about 6 carbon atoms in the chain, which is substituted with 1 to 5 fluoro groups. Non-limiting examples of suitable fluoroalkyl groups include fluoromethyl, trifluormethyl, fluoroethyl, and difluoroethyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Halogen” means fluorine, chlorine, bromine, or iodine. Preferred are fluorine, chlorine and bromine.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —SF₅, —OSF₅ (for aryl), —O—C(O)-alkyl, —O—C(O)-aryl, —O—C(O)-cycloalkyl, —C(═N—CN)—NH₂, —C(═NH)—NH₂, —C(═NH)—NH(alkyl), oxime (e.g., ═N—OH), —NY₁Y₂, -alkyl-NY₁Y₂, —C(O)NY₁Y₂, —SO₂NY₁Y₂ and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

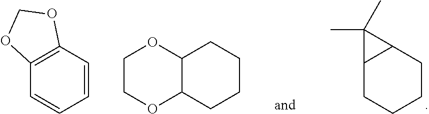

“Heteroarylaryl” means a heteroaryl moiety as defined herein linked or fused to an aryl moiety (defined above). Non-limiting examples of suitable heteroarylaryl include quinazolinyl, phthalazinyl, cinnolinyl, benzthiazolyl, indolyl, benzofuranyl, quinolinylmethyl and the like.

“Heterocyclylheteroaryl” means a heterocyclyl moiety as defined herein fused to a heteroaryl moiety as defined herein. A non-limiting example of a suitable heterocyclylheteroaryl is indolinyl.

“Heterocyclyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” also includes heterocyclyl rings as described above wherein ═O replaces two available hydrogens on the same ring carbon atom. An example of such a moiety is pyrrolidone:

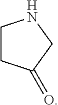

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” also includes heterocyclenyl rings as described above wherein ═O replaces two available hydrogens on the same ring carbon atom. Example of such moiety is pyrrolidinone:

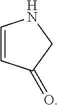

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

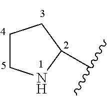 [SMILES: *C1CCCN1]

there is no —OH attached directly to carbons marked 2 and 5.

It should also be noted that tautomeric forms such as, for example, the moieties:

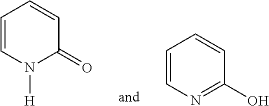 [SMILES: O=c1cccc[nH]1 ; Oc1ccccn1]

are considered equivalent in certain embodiments of this invention.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Alkoxy” or “Alkoxyl” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like) in sufficient purity to be characterizable by standard analytical techniques described herein or well known to the skilled artisan.

The present invention further includes the compound of formula I in all its isolated forms. Thus, for example, the compound of Formula I is intended to encompass all forms of the compound such as, for example, any solvates, hydrates, stereoisomers, tautomers etc.

The present invention further includes the compound of formula I in its purified form.

It should also be noted that any carbon as well as heteroatom with unsatisfied valences in the text, schemes, examples and Tables herein is assumed to have the sufficient number of hydrogen atom(s) to satisfy the valences. And any one or more of these hydrogen atoms can be deuterium.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula I, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C1-C2)alkyl and piperidino-, pyrrolidino- or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Compounds of Formula I, and salts, solvates, esters and prodrugs thereof, may exist in their tautomeric form (for example, as an amide or imino ether). All such tautomeric forms are contemplated herein as part of the present invention.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention. In addition, the present invention embraces all geometric isomers. For example, if a compound of Formula (I) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC columns.

It is also possible that the compounds of Formula (I) may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

In the compounds of this invention, the atoms may exhibit their natural isotopic abundances, or one or more of the atoms may be artificially enriched in a particular isotope having same atomic number, but an atomic mass or mass number different from the atomic mass or mass number predominantly found in nature. The present invention is meant to include all suitable isotopic variations of the compounds of generic Formula I. For example different isotopic forms of hydrogen (H) include protium (1H) and deuterium (2H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Examples herein using appropriate isotopically enriched reagents and/or intermediates. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Polymorphic forms of the compounds of Formula I, and of the salts, solvates, esters and prodrugs of the compounds of Formula I, are intended to be included in the present invention.

Those skilled in the art will appreciate that for some of the compounds of the invention, one isomer will show greater pharmacological activity than other isomers.

For preparing pharmaceutical compositions from the compounds described for use in the methods of this invention, inert, pharmaceutically acceptable carriers can be either solid or liquid. Solid form preparations include powders, tablets, dispersible granules, capsules, cachets and suppositories. The powders and tablets may be comprised of from about 5 to about 70 percent active ingredient. Suitable solid carriers are known in the art, e.g., magnesium carbonate, magnesium stearate, talc, sugar, lactose. Tablets, powders, cachets and capsules can be used as solid dosage forms suitable for oral administration.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Liquid form preparations include solutions, suspensions and emulsions. As an example may be mentioned water or water-propylene glycol solutions for parenteral injection.

Liquid form preparations may also include solutions for intranasal administration.

Aerosol preparations suitable for inhalation may include solutions and solids in powder form, which may be in combination with a pharmaceutically acceptable carrier, such as an inert compressed gas.

Also included are solid form preparations which are intended to be converted, shortly before use, to liquid form preparations for either oral or parenteral administration. Such liquid forms include solutions, suspensions and emulsions.

The compounds for use in the present invention may also be deliverable transdermally. The transdermal compositions can take the form of creams, lotions, aerosols and/or emulsions and can be included in a transdermal patch of the matrix or reservoir type as are conventional in the art for this purpose.

Preferably the compound of the invention is administered orally.

Preferably, the pharmaceutical preparation is in unit dosage form. In such form, the preparation is subdivided into unit doses containing appropriate quantities of the active component, e.g., an effective amount to achieve the desired purpose.

The quantity of active compound of the invention in a unit dose of preparation may be varied or adjusted from about 0.1 mg to 1000 mg, more preferably from about 1 mg to 300 mg, according to the particular application.

The actual dosage employed may be varied depending upon the requirements of the patient and the severity of the condition being treated. Determination of the proper dosage for a particular situation is within the purview of those skilled in the art. Generally, treatment is initiated with smaller dosages which are less than the optimum dose of the compound. Thereafter, the dosage is increased by small increments until the optimum effect under the circumstances is reached. For convenience, the total daily dosage may be divided and administered in portions during the day if desired.

The amount and frequency of administration of the compounds of the invention will be regulated according to the judgment of the attending clinician considering such factors as age, condition and size of the patient as well as severity of the symptoms being treated. A typical recommended dosage regimen for compounds of the invention is oral administration of from 10 mg to 2000 mg/day preferably 10 to 1000 mg/day, in two to four divided doses to provide relief from the diseases or conditions listed above.

The doses and dosage regimen of the other agents used in the treatment of diseases or conditions listed above will be determined by the attending clinician in view of the approved doses and dosage regimen in the package insert, taking into consideration the age, sex and condition of the patient and the severity of the disease. When administered in combination, the compound(s) of the invention and the other agent(s) for treating diseases or conditions listed above can be administered simultaneously or sequentially. This is particularly useful when the components of the combination are preferably given on different dosing schedules, e.g., one component is administered once daily and another every six hours, or when the preferred pharmaceutical compositions are different, e.g., one is preferably a tablet and one is a capsule. A kit comprising the separate dosage forms is therefore advantageous.

The compounds of the invention can be made according to the processes described below. The compounds of this invention are also exemplified in the examples below, which examples should not be construed as limiting the scope of the disclosure. Alternative mechanistic pathways and analogous structures within the scope of the invention may be apparent to those skilled in the art.

The following abbreviations have the following meanings unless defined otherwise: BF₃.Et₂O: boron trifluoride diethyl etherate; CMA: 80:18:2 chloroform/methanol/concentrated ammonium hydroxide; DIPEA: diisopropylethylamine; EDC.HCl: N-(3-dimethylaminopropyl)-N′-ethyl-carbodiimide hydrochloride; Et₂O: diethyl ether; EtOAc: ethyl acetate; EtOH: ethanol; HOBt: 1-hydroxybenzotriazole; Int.: Intermediate; MeOH: methanol; Na(OAc)₃BH: sodium triacetoxyborohydride; TLC: thin layer chromatography; TMG: 1,1,3,3-tetramethylguanidine.

### General Methods

The compounds of this invention can be made according to the processes described below.

### General Synthetic Scheme

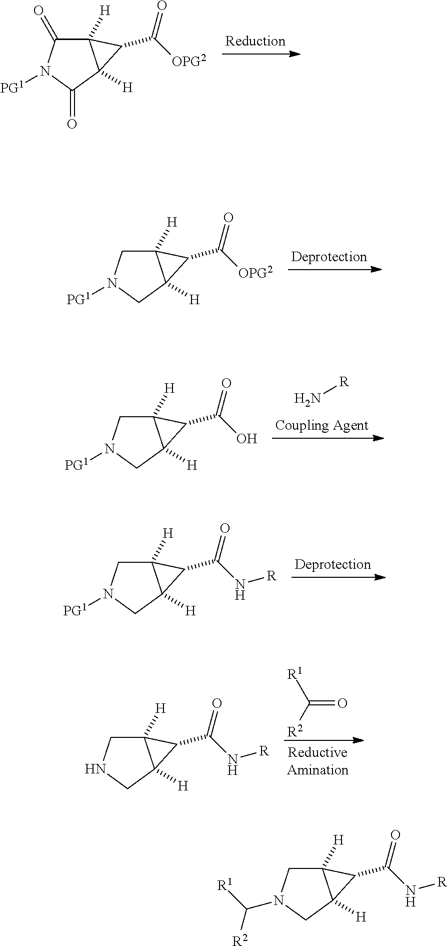

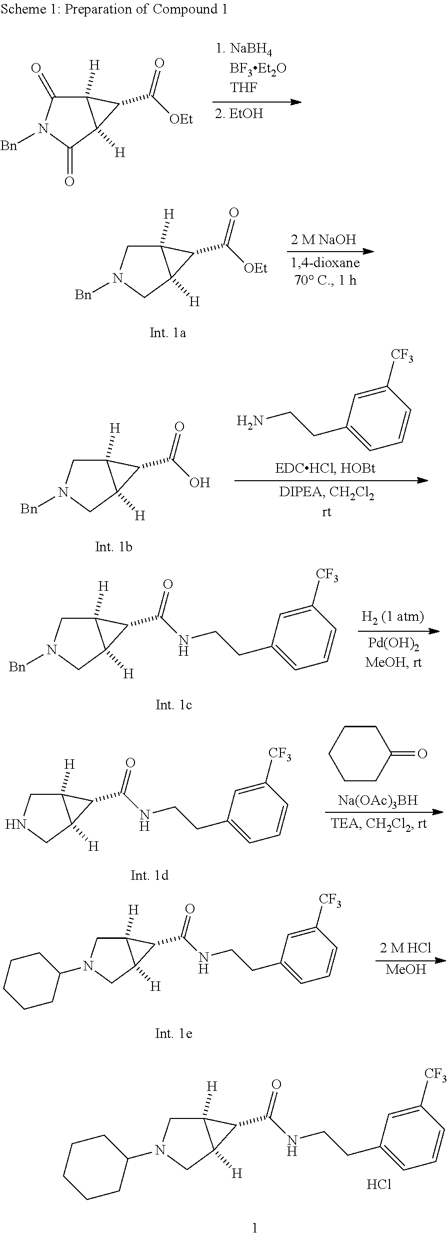

### Step A—Preparation of Compound Intermediate 1a

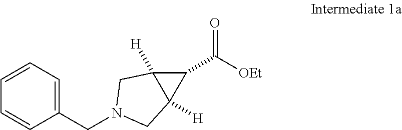 [SMILES: CCOC(=O)[C@H]1[C@@H]2CN(Cc3ccccc3)C[C@@H]21]

To a solution of sodium borohydride (4.50 g, 0.119 mol) in THF (110 mL) at 0° C. was added BF₃.Et₂O (19.6 mL, 0.156 mol) dropwise over 5-7 min. To the reaction mixture was added (α,α,α)-ethyl 3-benzyl-2,4-dioxo-3-azabicyclo[3.1.0]hexane-6-carboxylate [prepared as described in Synlett 1996, 1097] (11.05 g, 40.4 mmol) in THF (70 mL) via syringe pump and stirred overnight. The reaction mixture was cooled to 0° C. and EtOH (150 mL) was added slowly (due to gas evolution). The reaction mixture was warmed to room temperature over 5 h, then refluxed for 3 h, cooled to room temperature and concentrated in vacuo. The crude mixture was taken up in H₂O (150 mL) and CHCl₃ (250 mL). The aqueous layer was washed with CHCl₃ (2×250 mL) and the combined organic phase was dried (MgSO₄), filtered and concentrated in vacuo to yield the desired Intermediate 1a (8.92 g) as a thick yellow oil. LRMS: (M+H)⁺=246.1.

### Step B—Preparation of Compound Intermediate 1b

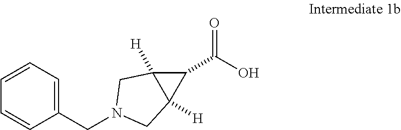 [SMILES: O=C(O)[C@H]1[C@@H]2CN(Cc3ccccc3)C[C@@H]21]

To a solution of compound Intermediate 1a (0.50 g, 2.04 mmol) in 1,4-dioxane (12 mL) was added 2 M NaOH (4.10 mL, 8.16 mmol) and the reaction stirred at room temperature overnight. TLC showed 25% starting material remained, so the reaction was heated to 70° C. for 1 h then stirred at room temperature for 72 h. Upon reaction completion, the 1,4-dioxane was removed in vacuo. The crude residue was neutralized using 4 N HCl (2.5 mL) to pH ˜5 and concentrated in vacuo to yield the desired Intermediate 1b (1.04 g; containing NaCl), which was used directly without further purification. LRMS: (M+H)⁺=218.1.

### Step C—Preparation of Compound Intermediate 1c

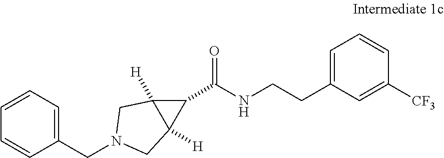 [SMILES: O=C(NCCc1cccc(C(F)(F)F)c1)[C@H]1[C@@H]2CN(Cc3ccccc3)C[C@@H]21]

To a solution of compound Intermediate 1b (0.44 g, 2.04 mmol) in CH₂Cl₂ (20 mL) was added DIPEA (2.50 mL, 14.30 mmol), HOBt (0.27 g, 2.04 mmol) and 2-(3-(trifluoromethyl)phenyl)ethanamine (0.42 g, 2.24 mmol). The mixture was stirred for 10 min then EDC.HCl (0.58 g, 3.06 mmol) was added and the reaction mixture stirred at room temperature overnight. Upon completion, the mixture was quenched with NaHCO₃ (˜30 mL) and diluted with CHCl₃ (˜100 mL). The organic layer was separated and the aqueous phase was then washed with CHCl₃ (2×40 mL). The combined organic phase was dried (MgSO₄), filtered and concentrated in vacuo. The crude residue was purified by flash chromatography on an ISCO Redi-Sep 80 g column using 0-10% CMA/CH₂Cl₂ as eluent to yield the desired Intermediate 1c (621 mg). LRMS: (M+H)⁺=389.1.

### Step D—Preparation of Compound Intermediate 1d

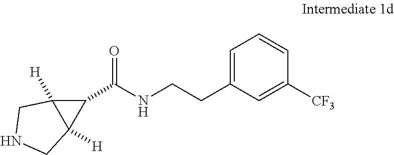 [SMILES: O=C(NCCc1cccc(C(F)(F)F)c1)[C@H]1[C@@H]2CNC[C@@H]21]

To a solution of compound Intermediate 1c (0.62 g, 1.59 mmol) in MeOH (20 mL) was added Pd(OH)₂ (0.15 g, 25 wt %). The mixture was purged with nitrogen for several minutes then stirred under a hydrogen atmosphere (15 psi) overnight. Upon completion, the mixture was purged with nitrogen then filtered through Celite®. The crude residue was purified by flash chromatography on an ISCO Redi-Sep column using 0-50% CMA/CH₂Cl₂ as eluent to yield the desired Intermediate 1d (442 mg). LRMS: (M+H)⁺=299.1.

### Step E—Preparation of Compound Intermediate 1e

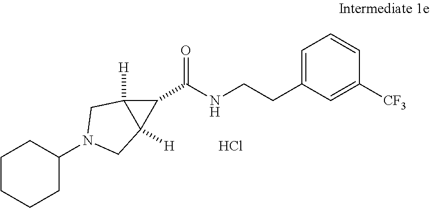

To a solution of compound Intermediate 1d (40 mg, 0.13 mmol) in CH₂Cl₂ (5.0 mL) and MeOH (2-3 drops) was added cyclohexanone (39 mg, 0.40 mmol) and TEA (94 μL, 0.67 mmol). The mixture was stirred for 45 min at room temperature and Na(OAc)₃BH (85 mg, 0.40 mmol) was added. The mixture was stirred at room temperature for 72 h. The crude mixture was diluted with CHCl₃ (30 mL) and washed with NaHCO₃ (20 mL). The aqueous phase was then washed with CHCl₃ (2×30 mL). The combined organic phase was dried (Na₂SO₄) and concentrated in vacuo. The crude residue was purified by flash chromatography on an ISCO Redi-Sep column using 0-15% CMA/CH₂Cl₂ as eluent to yield the desired Intermediate 1e (41 mg) as a white solid. LRMS: (M+H)⁴=381.4.

### Step F—Preparation of Compound 1

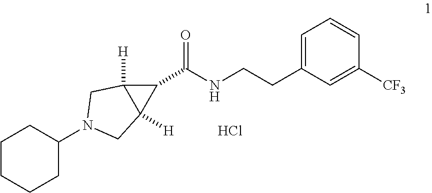

To a solution of Intermediate 1e (41.0 mg, 0.11 mmol) in MeOH (4.0 mL) was added HCl (2 M in diethyl ether, 260 μL, 0.52 mmol). The resulting mixture was allowed to stir at room temperature for 1 h. The mixture was concentrated in vacuo to yield the desired product 1 (30 mg) as a light brown solid. LRMS: (M+H)⁺381.4.

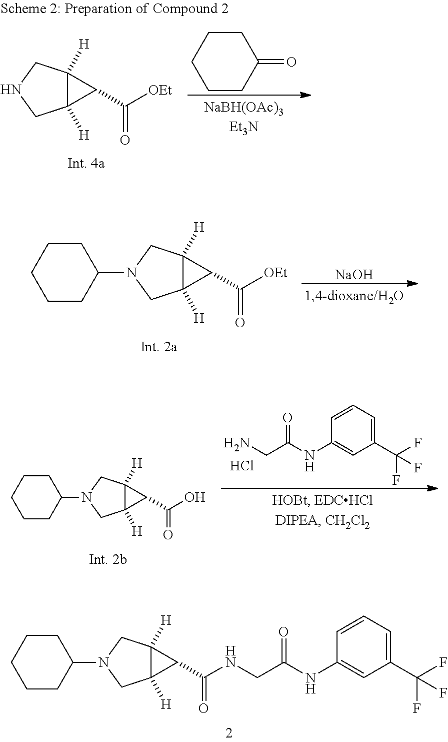

### Step A—Preparation of Compound Intermediate 2a

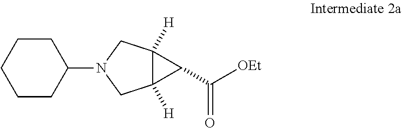 [SMILES: CCOC(=O)[C@H]1[C@@H]2CN(C3CCCCC3)C[C@@H]21]

To a stirred solution of Intermediate 4a [see Preparation of Compound 4; Step A] (39 mg, 0.24 mmol), cyclohexanone (73 mg, 0.74 mmol) and Et₃N (100 mg, 0.98 mmol) in CH₂Cl₂ (6 mL) was added NaBH(OAc)₃ (158 mg, 0.74 mmol). This mixture was stirred overnight at room temperature, then diluted with CH₂Cl₂ (50 mL) and washed with saturated NaHCO₃ solution (50 mL). The organic layer was dried (Na₂SO₄), filtered and concentrated to yield crude Intermediate 2a (120 mg) as a viscous oily residue, which was used in the next step without further purification. MS (APCI): (M+H)⁺=238.3.

### Step B—Preparation of Compound Intermediate 2b

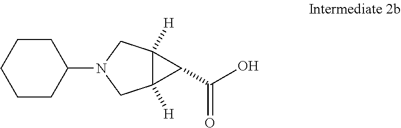 [SMILES: O=C(O)[C@H]1[C@@H]2CN(C3CCCCC3)C[C@@H]21]

To a solution of Intermediate 2a [120 mg, 0.24 mmol (assumed)] in 1,4-dioxane (6 mL) was added NaOH (25 mg, 0.62 mmol) in water (2 mL). This mixture was stirred overnight at room temperature, then concentrated to dryness. The resulting reaction mixture was diluted with water (5 mL) and washed with ethyl acetate. The aqueous solution was neutralized with 1 N HCl (pH 4-5), then concentrated to dryness to yield Intermediate 2b (75 mg) as a white solid, which was used in the next step without further purification. MS (APCI): (M+H)⁺=210.2.

### Step C—Preparation of Compound 2

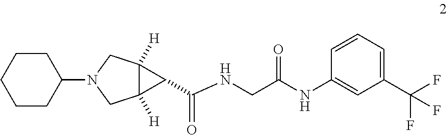 [SMILES: O=C(CNC(=O)[C@H]1[C@@H]2CN(C3CCCCC3)C[C@@H]21)Nc1cccc(C(F)(F)F)c1]

To a mixture of 2-amino-N-(3-(trifluoromethyl)phenyl)acetamide hydrochloride [prepared as described in WO 2005/039506] (60 mg, 0.24 mmol), Intermediate 2b [75 mg, 0.24 mmol (assumed)], HOBt (48 mg, 0.35 mmol) and DIPEA (152 mg, 1.18 mmol) in CH₂Cl₂ (6 mL) was added EDC.HCl (68 mg, 0.35 mmol). This mixture was stirred overnight at room temperature, diluted with CH₂Cl₂ (50 mL) and washed with saturated NaHCO₃ solution (50 mL). The organic phase was dried (Na₂SO₄), filtered, concentrated and purified by flash chromatography on an ISCO 12 g Redi-Sep column using 30% CMA (80:18:2 chloroform/methanol/ammonium hydroxide)/CH₂Cl₂ as an eluent to yield the desired product 2 [50 mg] as a white solid. MS (APCI): (M+H)⁺=410.2.

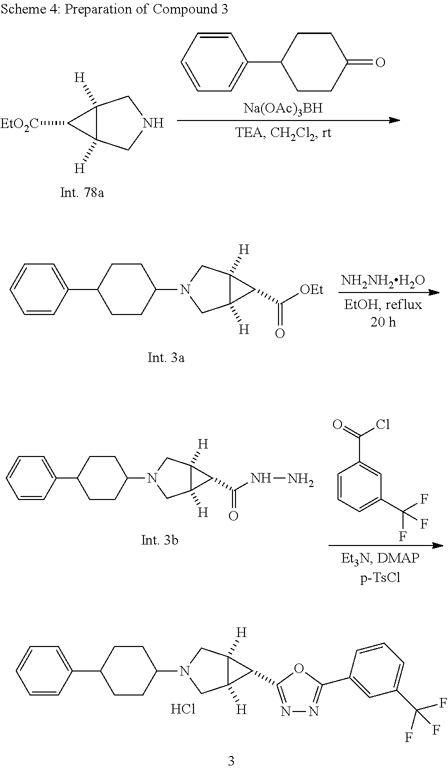

### Step A—Preparation of Compound Intermediate 3a

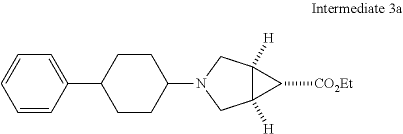 [SMILES: CCOC(=O)[C@H]1[C@@H]2CN(C3CCC(c4ccccc4)CC3)C[C@@H]21]

To a solution of Intermediate 4a [see Preparation of Compound 4; Step A] (3.15 g, 20.3 mmol) in CH₂Cl₂ (150 mL) was added 4-phenylcyclohexanone (6.19 g, 35.5 mmol) and TEA (8.21 g, 81.2 mmol). The mixture was stirred for 1 h at room temperature and Na(OAc)₃BH (7.53 g, 35.5 mmol) was added. The reaction was stirred at room temperature for 18 h. The reaction mixture was quenched with saturated aqueous NaHCO₃ (˜150 mL) and extracted with CHCl₃ (3×150 mL). The combined organic phase was dried (MgSO₄), filtered and concentrated in vacuo to yield a waxy yellow solid. The crude residue (˜9.5 g) was purified by flash chromatography on an ISCO Redi-Sep 220 g column using 0-20% EtOAc/hexanes as eluent to yield the desired product Intermediate 3a (3.30 g) as a light yellow oil. MS (Multimode): (M+H)⁺=314.2.

### Step B—Preparation of Compound Intermediate 3b

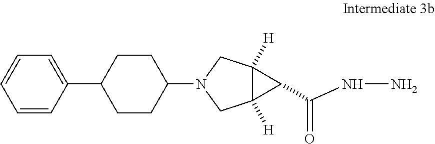 [SMILES: NNC(=O)[C@H]1[C@@H]2CN(C3CCC(c4ccccc4)CC3)C[C@@H]21]

To a solution of Intermediate 3a (2.06 g, 6.58 mmol) in EtOH (60 mL) was added aqueous NH₂NH₂.H₂O (35 wt % solution, 10 mL, 110 mmol). This mixture was heated at 80° C. for 20 h, then concentrated and diluted with CH₂Cl₂ (50 mL). The organic layer was washed with water (50 mL) and brine (50 mL), then dried (Na₂SO₄), filtered and concentrated to yield Intermediate 3b (1.9 g) as a white solid, which was used in the next step without further purification. ¹H NMR (CDCl₃) δ 7.31-7.16 (m, 5H), 6.97 (s, 1H), 4.12 (bs, 2H), 3.11 (d, J=8.95 Hz, 2H), 2.58-2.53 (m, 1H), 2.32 (m, 3H), 1.99 (bs, 2H), 1.88-1.74 (m, 5H), 1.59-1.49 (m, 4H).

### Step C—Preparation of Compound 3

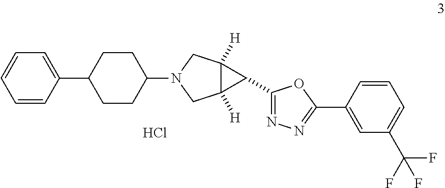

To a stirred solution of Intermediate 3b (108 mg, 0.36 mmol) in CH₂Cl₂ (25 mL) was added 3-(trifluoromethyl)benzoyl chloride (110 mg, 0.53 mmol). The resulting mixture was stirred at room temperature for 3 h. To the mixture was added DMAP (10 mg, 0.08 mmol), Et₃N (181 mg, 1.79 mmol) and tosyl chloride (170 mg, 0.89 mmol). After stirring overnight at room temperature, the reaction mixture was diluted with CH₂Cl₂ (50 mL) and washed with saturated NaHCO₃ solution (50 mL). The organic phase was dried (Na₂SO₄), filtered, concentrated and purified by flash chromatography on an ISCO 12 g column using 0-20% EtOAc/hexanes as an eluent to give the pure free base of compound 3 (50 mg). The free base of compound 3 was further treated with HCl (2 M in diethyl ether, 400 μL, 8.00 mmol) to yield desired compound 3 as a light brown solid (64 mg). MS (APCI): (M+H)⁺=454.2.

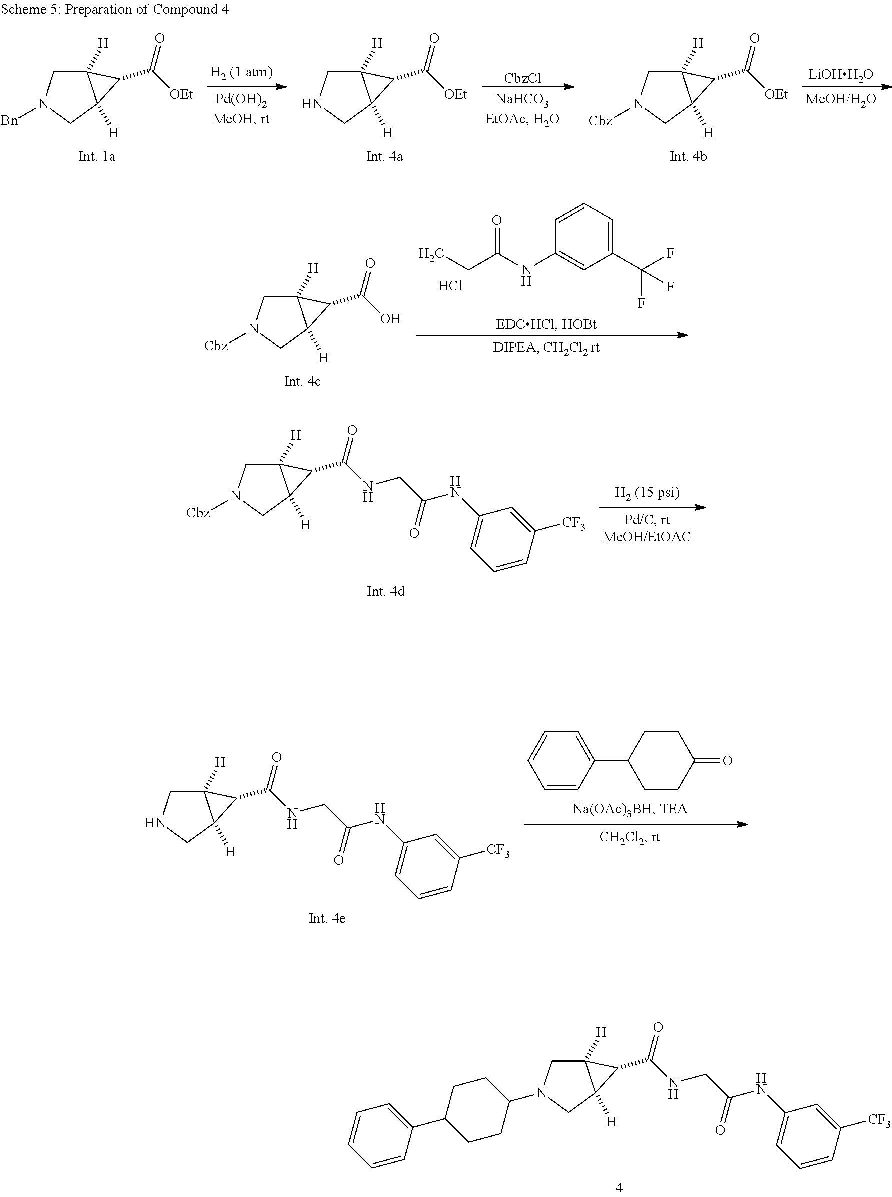

### Step A—Preparation of Compound Intermediate 4a

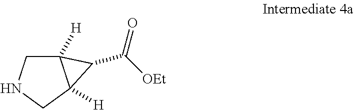

A solution of Intermediate 1a [see preparation of Compound 1; Step A] (4.53 g, 18.5 mmol) in MeOH (70 mL) was purged with N₂ for 5 min. To the reaction mixture was added Pd(OH)₂ (20 wt %, 1.15 g) and the reaction was purged with N₂ for another 5 min, then stirred under a H₂ atmosphere (15 psi) overnight. The reaction mixture was purged with N₂, filtered through Celite®, and washed with MeOH (100 mL) and EtOAc (100 mL). The reaction mixture was concentrated in vacuo to afford the desired Intermediate 4a (239 g) as a clear oil, which was used directly without further purification. ¹H NMR (CDCl₃) δ 4.11 (q, J=7.0 Hz, 2H), 3.07 (d, J=11.5 Hz, 2H), 2.96 (dd, J=12.5, 0.5 Hz, 2H), 2.00 (m, 2H), 1.65 (br s, 1H), 1.44 (t, J=3.0 Hz, 1H), 1.26 (t, J=7.0 Hz, 3H).

### Step B—Preparation of Compound Intermediate 4b

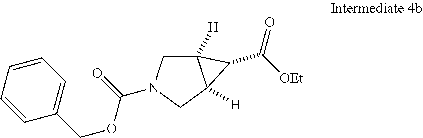

To a suspension of Intermediate 4a (2.79 g, 18.0 mmol) in 1:1 dioxane/H₂O (100 mL) was added NaHCO₃ (6.80 g, 80.9 mmol) and benzyl chloroformate (3.06 g, 18.0 mmol). After stirring the reaction mixture overnight, EtOAc (200 mL) and H₂O (50 mL) were added. The organic layer was separated and the aqueous layer was reextracted with EtOAc (2×200 mL). The combined organic layer was dried (MgSO₄), filtered and concentrated to yield the desired Intermediate 4b (5.08 g) as a light yellow oil, which was used directly without further purification. ¹H NMR (CDCl₃) δ 7.40-7.28 (m, 5H), 5.10 (s, 2H), 4.12 (q, J=7.0 Hz, 2H), 3.78-3.68 (m, 2H), 3.53-3.47 (m, 2H), 2.09 (s, 2H), 1.48 (t, J=3.0 Hz, 1H), 1.26 (t, J=7.0 Hz, 3H).

### Step C—Preparation of Compound Intermediate 4c

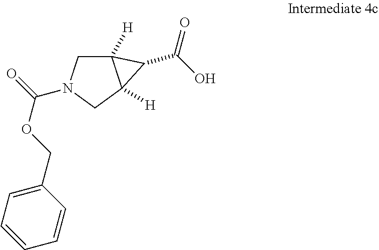

To a solution of Intermediate 4b (7.01 g, 24.2 mmol) in 1:1 MeOH/H₂O (240 mL) was added LiOH.H₂O (3.05 g, 72.6 mmol). The mixture was stirred at room temperature for 18 h. The MeOH was removed in vacuo, the resulting crude mixture was cooled to 0° C., then acidified to pH ˜3 using 4 M HCl (˜18 mL). The aqueous phase was washed with CH₂Cl₂(3×250 mL). The combined organic phase was dried (MgSO₄), filtered and concentrated in vacuo to yield desired Intermediate 4c (5.85 g). LRMS: (M+H)⁺=260.1.

### Step D—Preparation of Compound Intermediate 4d

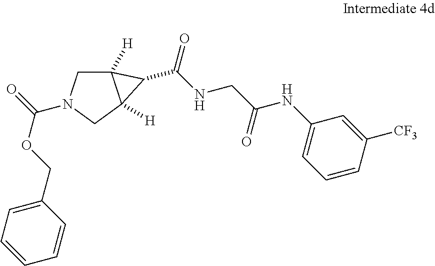

Using the same procedure as described for Compound 1, Step C; Intermediate 4c (4.70 g, 17.9 mmol) and 2-amino-N-(3-(trifluoromethyl)phenyl)acetamide hydrochloride [prepared as described in WO 2005/039506] (3.90 g, 17.9 mmol) yielded desired product Intermediate 4d (7.0 g) as a white solid. LRMS: (M+H)⁺=462.2.

### Step E—Preparation of Compound Intermediate 4e

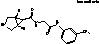

To a solution of compound Intermediate 4d (7.00 g, 15.2 mmol) in MeOH (400 mL) and EtOAc (200 mL) was added Pd/C (10 wt %, 0.700 g). The mixture was purged with nitrogen for several minutes, then stirred under a hydrogen atmosphere (15 psi) overnight. Upon completion, the mixture was purged with nitrogen, then filtered through Celite®. The crude residue was purified by flash chromatography on an ISCO Redi-Sep 120 g column using 0-100% CMA/CH₂Cl₂ as eluent to yield the desired Intermediate 4e (3.8 g). LRMS: (M+H)⁺=328.2.

### Step F—Preparation of Compound 4

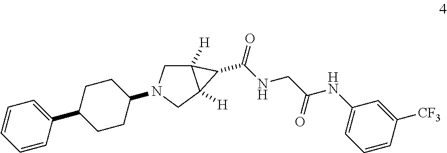

To a solution of compound Intermediate 4e (30 mg, 0.08 mmol) in CH₂Cl₂ (5.0 mL) was added 4-phenylcyclohexanone (43 mg, 0.25 mmol) and TEA (58 μL, 0.42 mmol). The mixture was stirred for 3 h at room temperature and Na(OAc)₃BH (53 mg, 0.25 mmol) was added. The mixture was stirred at room temperature for 18 h. The crude mixture was diluted with CHCl₃ (30 mL) and washed with NaHCO₃ (20 mL). The aqueous phase was then washed with CHCl₃ (2×30 mL). The combined organic phase was dried (Na₂SO₄) and concentrated in vacuo. The crude residue was purified by flash chromatography on an ISCO Redi-Sep 4 g column using 0-30% CMA/CH₂Cl₂ as eluent to yield the desired product 4 (4.2 mg) as a white solid, as well as the corresponding trans isomer, (1α, 5α, 6α)-N-(2-oxo-2-(3-trifluoromethyl)phenylamino)ethyl-3-((1β, 4α)-4-phenylcyclohexyl)-3-azabicyclo[3.1.0]hexane-6-carboxamide (0.7 mg). LRMS: (M+H)⁺=486.0.

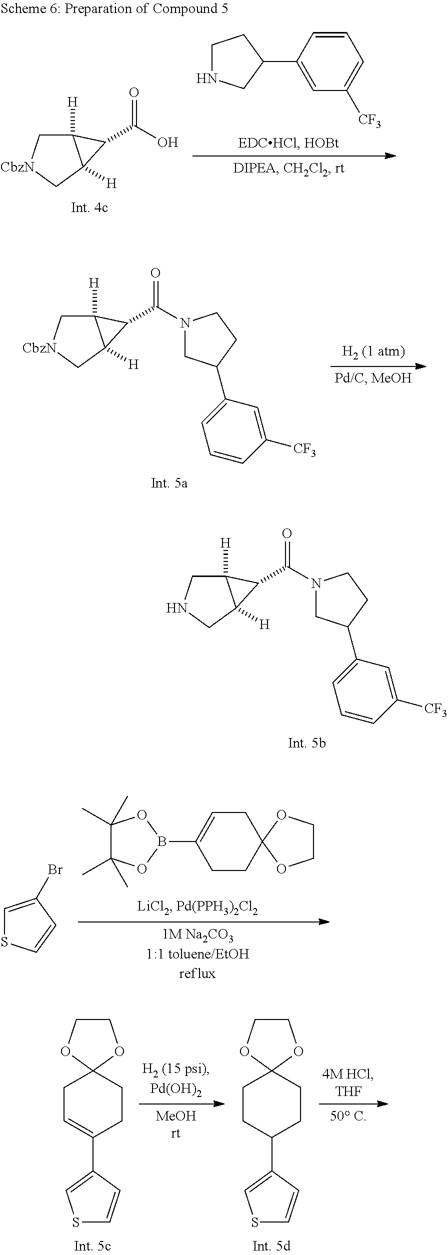

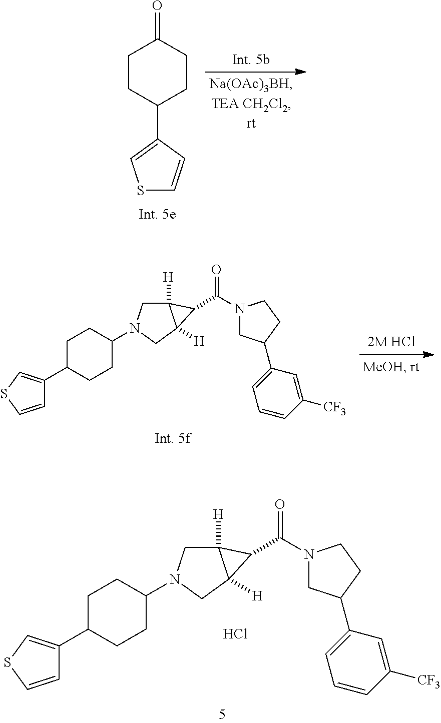 [SMILES: O=C1CCC(c2ccsc2)CC1 ; O=C([C@H]1[C@@H]2CN(C3CCC(c4ccsc4)CC3)C[C@@H]21)N1CCC(c2cccc(C(F)(F)F)c2)C1 ; O=C([C@H]1[C@@H]2CN(C3CCC(c4ccsc4)CC3)C[C@@H]21)N1CCC(c2cccc(C(F)(F)F)c2)C1 ; Cl]

### Step A—Preparation of Compound Intermediate 5a

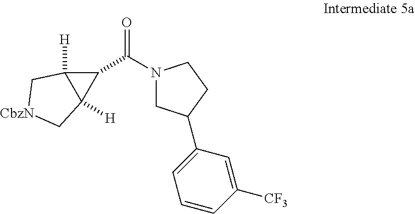 [SMILES: O=C(OCc1ccccc1)N1C[C@@H]2[C@H](C1)[C@H]2C(=O)N1CCC(c2cccc(C(F)(F)F)c2)C1]

Using the same procedure as described for Compound 1, Step C; Intermediate 4c [prepared in Example 78; Step C] (2.0 g, 7.6 mmol) and 3-(3-(trifluoromethyl)phenyl)pyrrolidine (1.9 g, 7.6 mmol) yielded desired Intermediate 5a (2.3 g) as a light yellow oil. LRMS: (M+H)⁺=460.3.

### Step B—Preparation of Compound Intermediate 5b

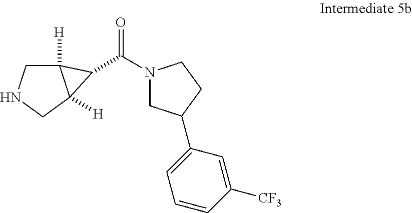

To a solution of compound Intermediate 5a (2.3 g, 5.0 mmol) in MeOH (150 mL) was added Pd/C (10 wt %, 0.23 g). The mixture was purged with nitrogen for several minutes then stirred under a hydrogen atmosphere (15 psi) overnight. Upon completion, the mixture was purged with nitrogen then filtered through Celite® and concentrated in vacuo to yield the desired Intermediate 5b (1.5 g). LRMS: (M+H)⁺=325.3.

### Step C—Preparation of Compound Intermediate 5c

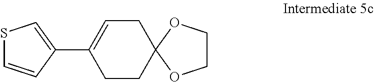 [SMILES: C1=C(c2ccsc2)CCC2(C1)OCCO2]

To a solution of 3-bromothiophene (0.540 g, 3.30 mmol) in 1:1 toluene/EtOH (5.0 mL) was added 4,4,5,5-tetramethyl-2-(1,4-dioxaspiro[4.5]dec-7-en-8-yl)-1,3,2-dioxaborolane (0.800 g, 3.00 mmol), lithium chloride (0.381 g, 9.00 mmol) and 1 M Na₂CO₃ (6.0 mL, 6.00 mmol). The mixture was purged with nitrogen for 5 min, then trans-dichlorobis(triphenylphosphine) palladium (II) (0.252 g, 0.360 mmol) was added. The mixture was again purged with nitrogen for 5 min and stirred at reflux for 24 h. The reaction mixture was quenched with H₂O (15 mL) and extracted with EtOAc (3×50 mL) and CHCl₃ (2×50 mL). The combined organic phase was dried (Na₂SO₄), filtered and concentrated in vacuo. The crude residue was purified by flash chromatography on an ISCO Redi-Sep 40 g column using 0-20% EtOAc/hexanes as eluent to yield the desired Intermediate 5c (528 mg) as a brown solid. LRMS: (M+H)⁺=222.8.

### Step D—Preparation of Compound Intermediate 5d

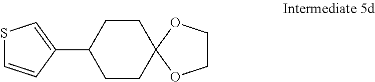 [SMILES: c1cc(C2CCC3(CC2)OCCO3)cs1]

To a solution of compound Intermediate 5c (0.525 g, 2.40 mmol) in MeOH (20 mL) was added Pd(OH)₂ (10 wt %, 0.05 g). The mixture was purged with nitrogen for several minutes then stirred under a hydrogen atmosphere (15 psi) overnight. Upon completion, the mixture was purged with nitrogen then filtered through Celite®. The filtrate was concentrated in vacuo to yield desired Intermediate 5d (494 mg) as a yellow oil. LRMS: (M+H)⁺=225.4.

### Step E—Preparation of Compound Intermediate 5e

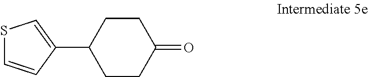 [SMILES: O=C1CCC(c2ccsc2)CC1]

To a solution of Intermediate 5d (0.492 g, 2.20 mmol) in THF (33 mL) was added 4 M HCl (38.0 mL, 154 mmol). The mixture was heated to 50° C. for 18 h and the cooled reaction mixture was quenched with solid Na₂CO₃ to pH ˜10-11 and extracted with CHCl₃ (3×50 mL). The organic phase was combined, dried (Na₂SO₄), filtered and concentrated in vacuo. The crude residue was purified by flash chromatography on an ISCO Redi-Sep 12 g column using 0-15% EtOAc/hexanes as eluent to yield the desired Intermediate 5e (215 mg) as a light yellow oil. LRMS: (M+H)⁺=181.0.

### Step F—Preparation of Compound Intermediate 5f

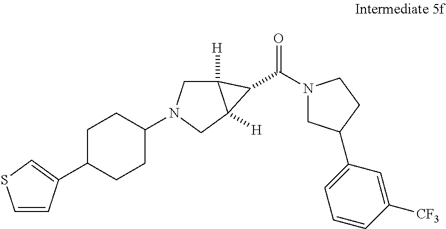

Using the same procedure as descried for Compound 1, Step E; Intermediate 5b (80.0 mg, 0.250 mmol) and Intermediate 5e (67.0 mg, 0.370 mmol) yielded crude Intermediate 5f. The crude residue was purified by flash chromatography on an ISCO Redi-Sep 4 g column using 0-5% CMA/CH₂Cl₂ as eluent to yield the desired Intermediate 5f (56 mg) as a clear oil, as well as the corresponding trans isomer, ((1α, 5α, 6α)-3-((1β, 4α)-4-(thiphen-3-yl)cyclohexyl)-3-azabicyclo[3.1.0]hexan-6-yl)(3-(3-trifluoromethyl)phenyl)pyrrolidin-1-yl) methanone (27 mg). LRMS: (M+H)⁺=489.5.

### Step G—Preparation of Compound 5

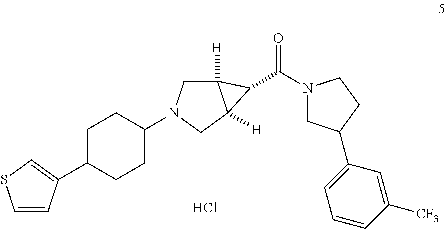

Using the same procedure as described for Compound 1, Step F; Intermediate 5f (56.0 mg, 0.11 mmol) and HCl (2 M in diethyl ether, 280 μL, 0.573 mmol) yielded the desired product 5 (51.4 mg) as a brown solid. LRMS: (M+H)⁺=489.8.

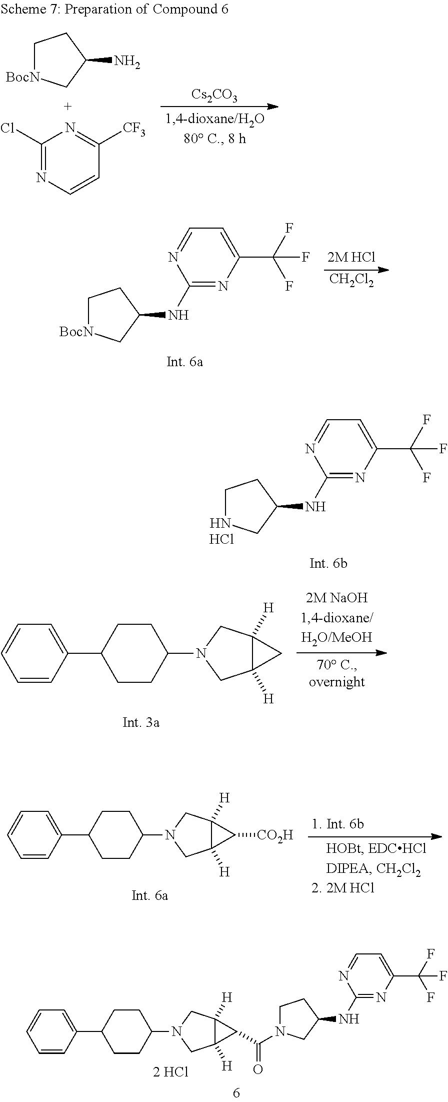

### Step A—Preparation of Compound Intermediate 6a

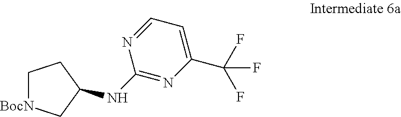 [SMILES: CC(C)(C)OC(=O)N1CC[C@@H](Nc2nccc(C(F)(F)F)n2)C1]

A mixture of (R)-tert-butyl-3-aminopyrrolidine-1-carboxylate (124 mg, 0.66 mmol), 2-chloro-4-(trifluoromethyl)pyrimidine (122 mg, 0.66 mmol), Cs₂CO₃ (870 mg, 2.67 mmol) in 1,4-dioxane and water (5/2 mL each) was heated at 80° C. for 8 h, diluted with CH₂Cl₂ (50 mL) and washed with water (50 mL). The organic phase was dried (Na₂SO₄), filtered and concentrated to yield the desired product Intermediate 6a (215 mg) as off-white solid, which was used in the next step without further purification. MS (APCI): (M+H-Boc)⁺=233.0.

### Step B—Preparation of Compound Intermediate 6b

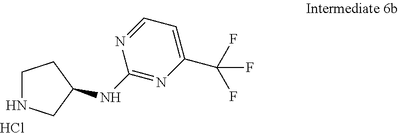 [SMILES: FC(F)(F)c1ccnc(N[C@@H]2CCNC2)n1 ; Cl]

To a stirred solution of Intermediate 6a (215 mg, 0.64 mmol) in CH₂Cl₂ (15 mL) was added HCl (2 M solution in diethyl ether, 5.0 mL, 10.0 mmol). This mixture was stirred at room temperature for 24 h, then concentrated to yield Intermediate 6b (210 mg) as off-white solid, which was used in the next step without further purification. MS (APCI): (M+H)⁺=233.3.

### Step C—Preparation of Compound Intermediate 6c

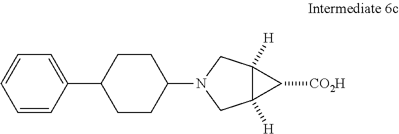 [SMILES: O=C(O)[C@H]1[C@@H]2CN(C3CCC(c4ccccc4)CC3)C[C@@H]21]

To a solution of Intermediate 3a [see Preparation of Compound 3; Step A] (3.25 g, 10.4 mmol) in 3:1 1,4-dioxane/water (100 mL) was added 2 M NaOH (20 mL, 41.6 mmol). The reaction was stirred overnight at room temperature. TLC indicated only starting material. LiOH monohydrate (1.31 g, 31.2 mmol) and MeOH (30 mL) were added and the reaction was heated to 70° C. overnight. The reaction mixture was concentrated to dryness, resuspended in water (100 mL) and acidified to pH 1 with 2 M HCl. The reaction mixture was concentrated to afford the desired Intermediate 6c, (8.55 g; NaCl salts present) as an off-white solid, which was used directly without further purification. ¹H NMR (CD₃OD) δ 7.27-7.15 (m, 4H), 7.15-7.06 (m, 1H), 3.14 (d, J=8.5 Hz, 2H), 2.53 (m, 1H), 2.32-2.17 (m, 3H), 1.95-1.80 (m, 7H), 1.57-1.42 (m, 4H).

### Step D—Preparation of Compound 6

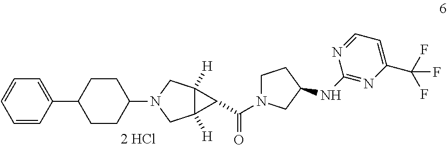 [SMILES: O=C([C@H]1[C@@H]2CN(C3CCC(c4ccccc4)CC3)C[C@@H]21)N1CC[C@@H](Nc2nccc(C(F)(F)F)n2)C1]

Using the same procedure as described for Compound 2, Step C; Intermediate 6b (105 mg, 0.31 mmol), Intermediate 6c [255 mg, 0.31 mmol (assumed; contained NaCl salt)] gave the pure free base of compound 6 (37 mg). The free base of compound 6 was further treated with HCl (2 M in diethyl ether, 100 μL, 0.20 mmol) to yield desired compound 6 as off-white solid (45 mg). MS (APCI): (M+H)⁺=500.5.

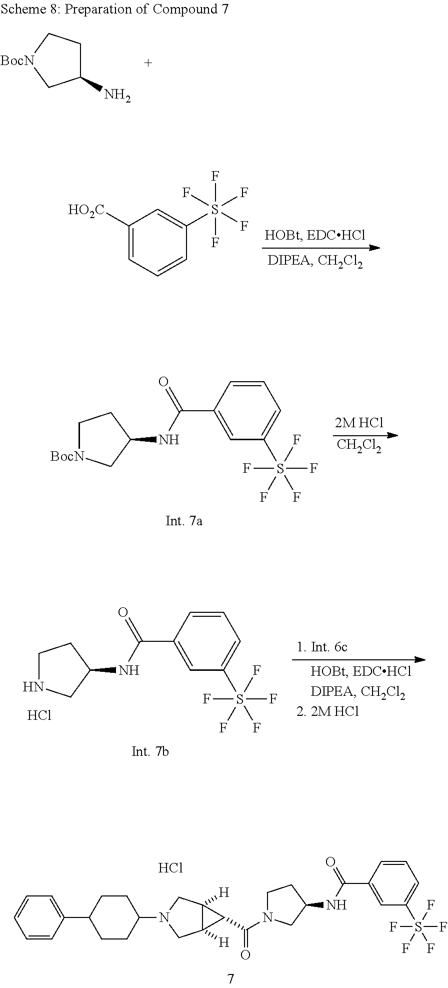

### Step A—Preparation of Compound Intermediate 7a

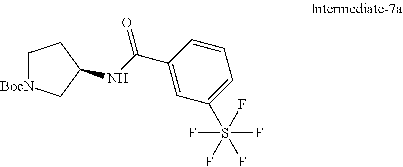 [SMILES: CC(C)(C)OC(=O)N1CC[C@@H](NC(=O)c2cccc(S(F)(F)(F)(F)F)c2)C1]

To a mixture of (R)-tert-butyl-3-aminopyrrolidine-1-carboxylate (58 mg, 0.30 mmol), 3-(pentafluorosulfur)benzoic acid (77 mg, 0.30 mmol), HOBt (62 mg, 0.45 mmol) and DIPEA (296 mg, 2.29 mmol) in CH₂Cl₂ (20 mL) was added EDC.HCl (89 mg, 0.46 mmol). This mixture was stirred overnight at room temperature, diluted with CH₂Cl₂ (50 mL) and washed with saturated NaHCO₃ solution (50 mL). The organic phase was dried (Na₂SO₄), filtered and concentrated to yield the desired product Intermediate 7a (135 mg) as a light brown solid, which was used in the next step without further purification. MS (APCI): (M+H-Boc)⁺=317.2.

### Step B—Preparation of Compound Intermediate 7b

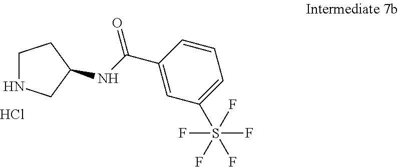 [SMILES: Cl ; O=C(N[C@@H]1CCNC1)c1cccc(S(F)(F)(F)(F)F)c1]

Using the same procedure as described for Compound 6, Step B; Intermediate 7a [135 mg, 0.30 mmol (assumed)] gave Intermediate 7b (113 mg) as a light brown solid, which was used in the next step without further purification. ¹H NMR (CDCl₃) δ 9.95 (m, 2H), 8.58 (d, J=7.55 Hz, 1H), 8.49 (s, 1H), 8.24 (d, J=7.75 Hz, 1H), 7.85 (dd, J=8.0 Hz, 1H), 7.52 (t, J=7.86 Hz, 1H), 5.0 (m, 1H), 3.62 (m, 2H), 3.47 (m, 2H), 2.25-2.49 (m, 2H).

### Step C—Preparation of Compound 7

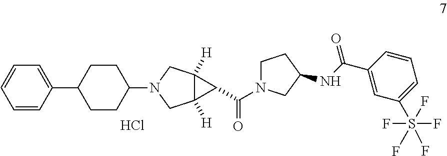 [SMILES: O=C(N[C@@H]1CCN(C(=O)[C@H]2[C@@H]3CN(C4CCC(c5ccccc5)CC4)C[C@@H]32)C1)c1cccc(S(F)(F)(F)(F)F)c1 ; Cl]

Using the same procedure as described for Compound 2, Step C; Intermediate 7b (113 mg, 0.30 mmol), Intermediate 6c [see preparation of Compound 6; Step C] [110 mg, 0.25 mmol (assumed; contained NaCl salts)] gave the pure free base of compound 7 (79 mg). The free base of compound 7 was further treated with HCl (2 M in diethyl ether, 100 μL, 0.20 mmol) to yield desired compound 7 as off-white solid (85 mg). MS (APCI): (M+H)⁺=584.4.

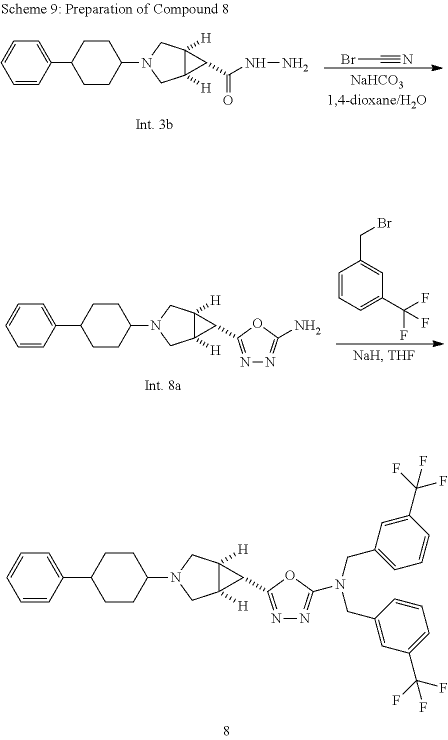

### Step A—Preparation of Compound Intermediate 8a

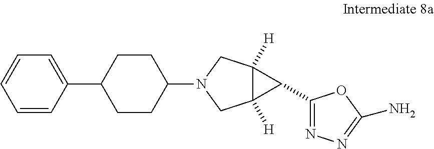 [SMILES: Nc1nnc([C@H]2[C@@H]3CN(C4CCC(c5ccccc5)CC4)C[C@@H]32)o1]

To a solution of Intermediate 3b (1.66 g, 5.54 mmol) in 1,4-dioxane (66 mL) was added NaHCO₃ (832 mg, 9.90 mmol) in water (20 mL), followed by cyanogen bromide (1.05 g, 9.91 mmol). This mixture was stirred at room temperature for 5 h, then diluted with CH₂Cl₂ (50 mL) and washed with saturated NaHCO₃ solution (50 mL) followed by brine (50 mL). The organic phase was dried (Na₂SO₄), filtered and concentrated to yield Intermediate 8a (1.75 g) as a white solid, which was used in the next step without further purification. MS (APCI): (M±H)⁺=325.6.

### Step B—Preparation of Compound 8

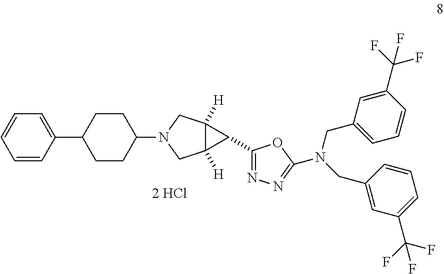 [SMILES: FC(F)(F)c1cccc(CN(Cc2cccc(C(F)(F)F)c2)c2nnc([C@H]3[C@@H]4CN(C5CCC(c6ccccc6)CC5)C[C@@H]43)o2)c1]

To a stirred solution of Intermediate 8a (131 mg, 0.4 mmol) in THF (10 mL) was added NaH (60 wt %, 19.6 mg, 0.49 mmol) followed by 3-(trifluoromethyl)benzyl bromide (101 mg, 0.42 mmol). This mixture was stirred at room temperature for 3.5 h. TLC indicated the reaction was complete. The reaction mixture was quenched with water and diluted with CH₂Cl₂ (50 mL). The organic phase was dried (Na₂SO₄), filtered and concentrated. The crude residue was purified by flash chromatography on an ISCO 12 g column using 30% CMA (80:18:2 chloroform/methanol/ammonium hydroxide)/CH₂Cl₂ as an eluent to yield the pure free base of compound 8 (5 mg). The free base of compound 8 was further treated with HCl (2 M in diethyl ether, 30 μL, 0.06 mmol) to yield desired compound 8 as a light yellow solid (6.5 mg). MS (APCI): (M+H)⁺=641.5.

Using procedures analogous to those described above, the compounds of table 1 were synthesized.

TABLE 1

| | | Mol. | LC-MS |
|---|---|---|---|
| No. | Mol. Structure | Wt | (M + H) |
| 1 | 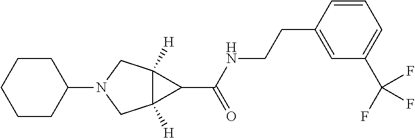 [SMILES: O=C(NCCc1cccc(C(F)(F)F)c1)C1[C@H]2CN(C3CCCCC3)C[C@@H]12] | 380.5 | 381.4 |
| 2 | 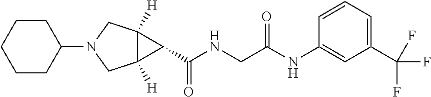 [SMILES: O=C(CNC(=O)[C@H]1[C@@H]2CN(C3CCCCC3)C[C@@H]21)Nc1cccc(C(F)(F)F)c1] | 409.5 | 410.3 |
| 3 | 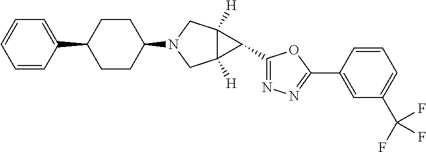 [SMILES: FC(F)(F)c1cccc(-c2nnc([C@H]3[C@@H]4CN([C@H]5CC[C@@H](c6ccccc6)CC5)C[C@@H]43)o2)c1] | 453.5 | 454.1 |
| 4 | 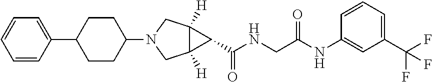 [SMILES: O=C(CNC(=O)[C@H]1[C@@H]2CN(C3CCC(c4ccccc4)CC3)C[C@@H]21)Nc1cccc(C(F)(F)F)c1] | 485.6 | 486.0 |
| 5 | 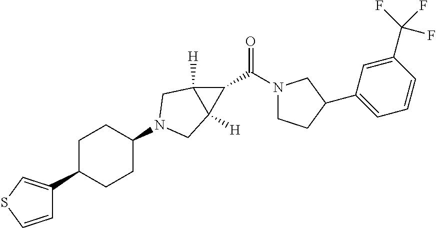 [SMILES: O=C([C@H]1[C@@H]2CN([C@H]3CC[C@@H](c4ccsc4)CC3)C[C@@H]21)N1CCC(c2cccc(C(F)(F)F)c2)C1] | 488.6 | 489.8 |
| 6 | 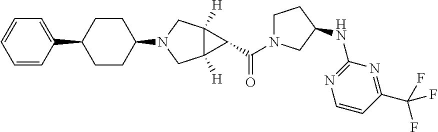 [SMILES: O=C([C@H]1[C@@H]2CN([C@H]3CC[C@@H](c4ccccc4)CC3)C[C@@H]21)N1CC[C@@H](Nc2nccc(C(F)(F)F)n2)C1] | 499.6 | 500.5 |
| 7 | 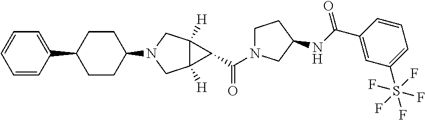 [SMILES: O=C(N[C@@H]1CCN(C(=O)[C@H]2[C@@H]3CN([C@H]4CC[C@@H](c5ccccc5)CC4)C[C@@H]32)C1)c1cccc(S(F)(F)(F)(F)F)c1] | 583.7 | 584.1 |
| 8 | 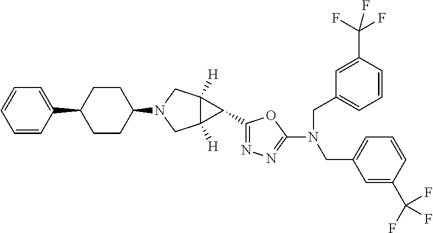 [SMILES: FC(F)(F)c1cccc(CN(Cc2cccc(C(F)(F)F)c2)c2nnc([C@H]3[C@@H]4CN([C@H]5CC[C@@H](c6ccccc6)CC5)C[C@@H]43)o2)c1] | 640.7 | 641.5 |
| 9 | 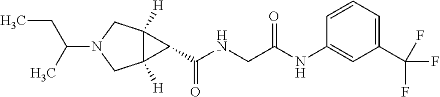 [SMILES: CCC(C)N1C[C@@H]2[C@H](C1)[C@H]2C(=O)NCC(=O)Nc1cccc(C(F)(F)F)c1] | 383.4 | 384.2 |
| 10 | 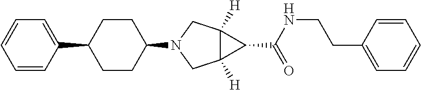 [SMILES: O=C(NCCc1ccccc1)[C@H]1[C@@H]2CN([C@H]3CC[C@@H](c4ccccc4)CC3)C[C@@H]21] | 388.6 | 389.3 |
| 11 | 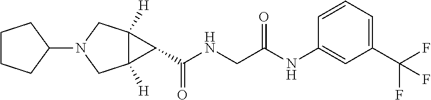 [SMILES: O=C(CNC(=O)[C@H]1[C@@H]2CN(C3CCCC3)C[C@@H]21)Nc1cccc(C(F)(F)F)c1] | 395.4 | 396.2 |
| 12 | 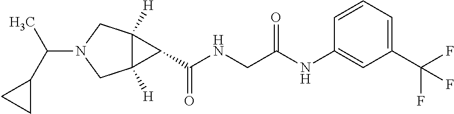 [SMILES: CC(C1CC1)N1C[C@@H]2[C@H](C1)[C@H]2C(=O)NCC(=O)Nc1cccc(C(F)(F)F)c1] | 395.4 | 396.3 |
| 13 | 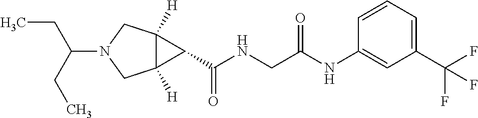 [SMILES: CCC(CC)N1C[C@@H]2[C@H](C1)[C@H]2C(=O)NCC(=O)Nc1cccc(C(F)(F)F)c1] | 397.4 | 398.1 |
| 14 | 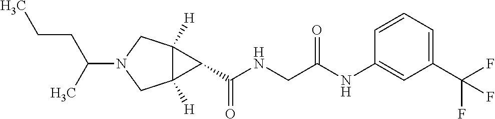 [SMILES: CCCC(C)N1C[C@@H]2[C@H](C1)[C@H]2C(=O)NCC(=O)Nc1cccc(C(F)(F)F)c1] | 397.4 | 398.2 |
| 15 | 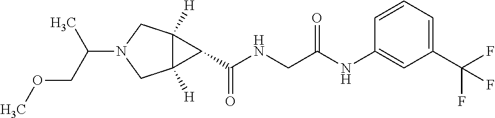 [SMILES: COCC(C)N1C[C@@H]2[C@H](C1)[C@H]2C(=O)NCC(=O)Nc1cccc(C(F)(F)F)c1] | 399.4 | 400.2 |
| 16 | 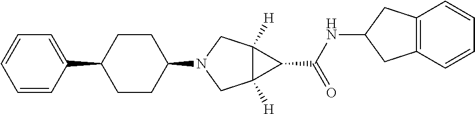 [SMILES: O=C(NC1Cc2ccccc2C1)[C@H]1[C@@H]2CN([C@H]3CC[C@@H](c4ccccc4)CC3)C[C@@H]21] | 400.6 | 401.9 |
| 17 | 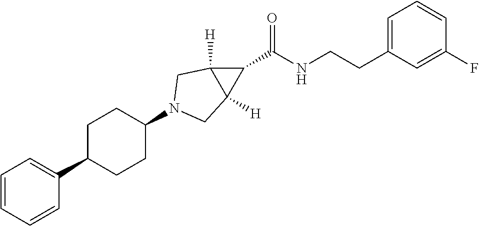 [SMILES: O=C(NCCc1cccc(F)c1)[C@H]1[C@@H]2CN([C@H]3CC[C@@H](c4ccccc4)CC3)C[C@@H]21] | 406.5 | 407.1 |
| 18 | 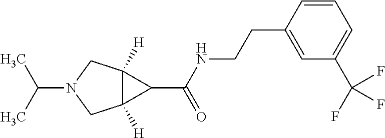 [SMILES: CC(C)N1C[C@@H]2C(C(=O)NCCc3cccc(C(F)(F)F)c3)[C@@H]2C1] | 340.4 | 341.0 |
| 19 | 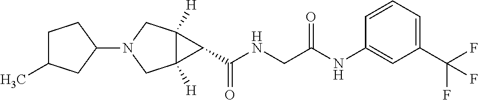 [SMILES: CC1CCC(N2C[C@@H]3[C@H](C2)[C@H]3C(=O)NCC(=O)Nc2cccc(C(F)(F)F)c2)C1] | 409.5 | 410.2 |
| 20 | 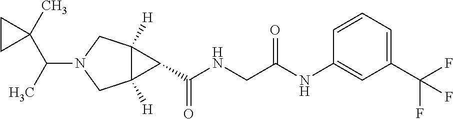 [SMILES: CC(N1C[C@@H]2[C@H](C1)[C@H]2C(=O)NCC(=O)Nc1cccc(C(F)(F)F)c1)C1(C)CC1] | 409.5 | 410.4 |
| 21 | 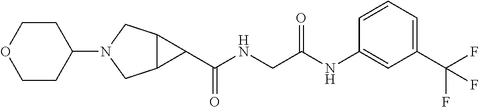 [SMILES: O=C(CNC(=O)C1C2CN(C3CCOCC3)CC21)Nc1cccc(C(F)(F)F)c1] | 411.4 | 412.5 |
| 22 | 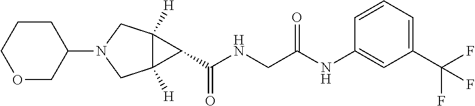 [SMILES: O=C(CNC(=O)[C@H]1[C@@H]2CN(C3CCCOC3)C[C@@H]21)Nc1cccc(C(F)(F)F)c1] | 411.4 | 412.2 |
| 23 | 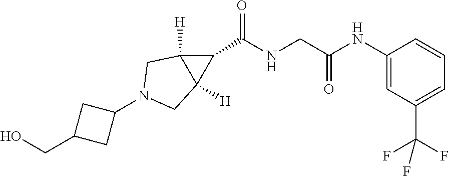 [SMILES: O=C(CNC(=O)[C@H]1[C@@H]2CN(C3CC(CO)C3)C[C@@H]21)Nc1cccc(C(F)(F)F)c1] | 411.4 | 412.4 |
| 24 | 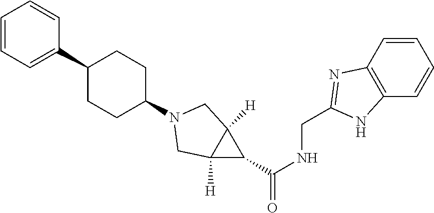 [SMILES: O=C(NCc1nc2ccccc2[nH]1)[C@H]1[C@@H]2CN([C@H]3CC[C@@H](c4ccccc4)CC3)C[C@@H]21] | 414.6 | 415.3 |
| 25 | 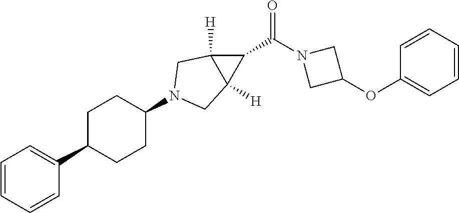 [SMILES: O=C([C@H]1[C@@H]2CN([C@H]3CC[C@@H](c4ccccc4)CC3)C[C@@H]21)N1CC(Oc2ccccc2)C1] | 416.6 | 417.3 |
| 26 | 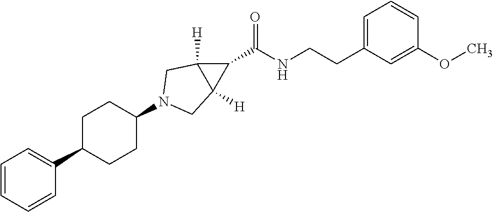 [SMILES: COc1cccc(CCNC(=O)[C@H]2[C@@H]3CN([C@H]4CC[C@@H](c5ccccc5)CC4)C[C@@H]32)c1] | 418.6 | 419.5 |
| 27 | 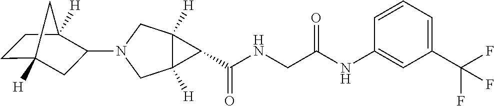 [SMILES: O=C(CNC(=O)[C@H]1[C@@H]2CN(C3C[C@@H]4CC[C@H]3C4)C[C@@H]21)Nc1cccc(C(F)(F)F)c1] | 421.5 | 422.6 |
| 28 | 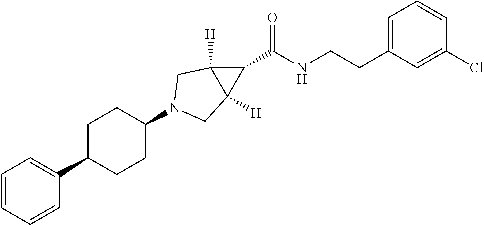 [SMILES: O=C(NCCc1cccc(Cl)c1)[C@H]1[C@@H]2CN([C@H]3CC[C@@H](c4ccccc4)CC3)C[C@@H]21] | 423.0 | 423.6 |
| 29 | 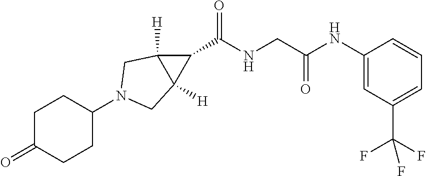 [SMILES: O=C1CCC(N2C[C@@H]3[C@H](C2)[C@H]3C(=O)NCC(=O)Nc2cccc(C(F)(F)F)c2)CC1] | 423.4 | 424.6 |
| 30 | 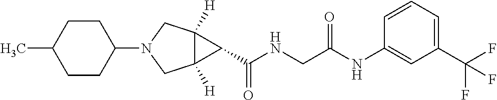 [SMILES: CC1CCC(N2C[C@@H]3[C@H](C2)[C@H]3C(=O)NCC(=O)Nc2cccc(C(F)(F)F)c2)CC1] | 423.5 | 424.2 |
| 31 | 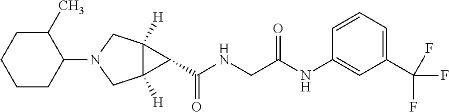 [SMILES: CC1CCCCC1N1C[C@@H]2[C@H](C1)[C@H]2C(=O)NCC(=O)Nc1cccc(C(F)(F)F)c1] | 423.5 | 424.2 |
| 32 | 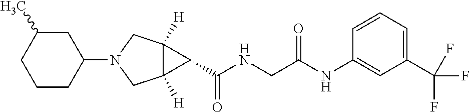 [SMILES: CC1CCCC(N2C[C@@H]3[C@H](C2)[C@H]3C(=O)NCC(=O)Nc2cccc(C(F)(F)F)c2)C1] | 423.5 | 424.2 |
| 33 | 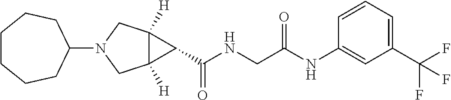 [SMILES: O=C(CNC(=O)[C@H]1[C@@H]2CN(C3CCCCCC3)C[C@@H]21)Nc1cccc(C(F)(F)F)c1] | 423.5 | 424.2 |
| 34 | 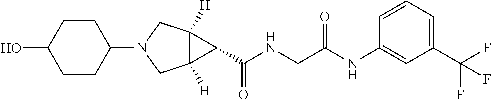 [SMILES: O=C(CNC(=O)[C@H]1[C@@H]2CN(C3CCC(O)CC3)C[C@@H]21)Nc1cccc(C(F)(F)F)c1] | 425.5 | 426.0 |
| 35 | 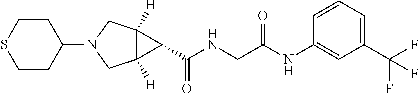 [SMILES: O=C(CNC(=O)[C@H]1[C@@H]2CN(C3CCSCC3)C[C@@H]21)Nc1cccc(C(F)(F)F)c1] | 427.5 | 428.4 |
| 36 | 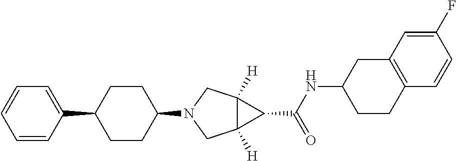 [SMILES: O=C(NC1CCc2ccc(F)cc2C1)[C@H]1[C@@H]2CN([C@H]3CC[C@@H](c4ccccc4)CC3)C[C@@H]21] | 432.6 | 433.9 |
| 37 | 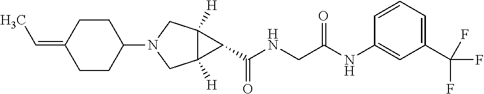 [SMILES: CC=C1CCC(N2C[C@@H]3[C@H](C2)[C@H]3C(=O)NCC(=O)Nc2cccc(C(F)(F)F)c2)CC1] | 435.5 | 436.2 |
| 38 | 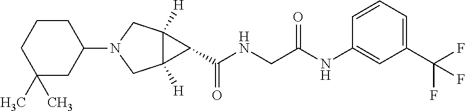 [SMILES: CC1(C)CCCC(N2C[C@@H]3[C@H](C2)[C@H]3C(=O)NCC(=O)Nc2cccc(C(F)(F)F)c2)C1] | 437.5 | 438.4 |
| 39 | 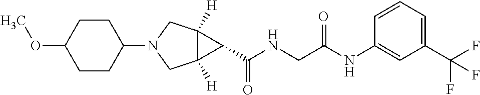 [SMILES: COC1CCC(N2C[C@@H]3[C@H](C2)[C@H]3C(=O)NCC(=O)Nc2cccc(C(F)(F)F)c2)CC1] | 439.5 | 440.2 |
| 40 | 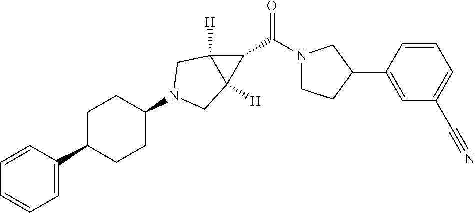 [SMILES: N#Cc1cccc(C2CCN(C(=O)[C@H]3[C@@H]4CN([C@H]5CC[C@@H](c6ccccc6)CC5)C[C@@H]43)C2)c1] | 439.6 | 440.3 |
| 41 | 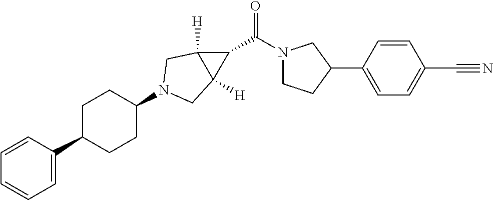 [SMILES: N#Cc1ccc(C2CCN(C(=O)[C@H]3[C@@H]4CN([C@H]5CC[C@@H](c6ccccc6)CC5)C[C@@H]43)C2)cc1] | 439.6 | 440.3 |
| 42 | 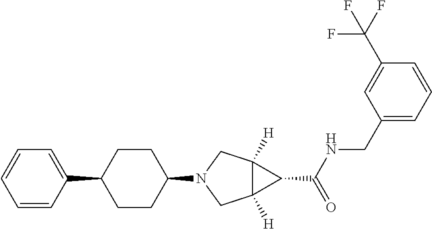 [SMILES: O=C(NCc1cccc(C(F)(F)F)c1)[C@H]1[C@@H]2CN([C@H]3CC[C@@H](c4ccccc4)CC3)C[C@@H]21] | 442.5 | 443.2 |
| 44 | 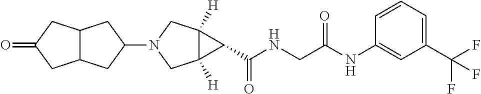 [SMILES: O=C1CC2CC(N3C[C@@H]4[C@H](C3)[C@H]4C(=O)NCC(=O)Nc3cccc(C(F)(F)F)c3)CC2C1] | 449.5 | 450.4 |
| 45 | 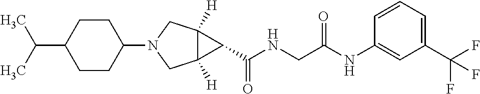 [SMILES: CC(C)C1CCC(N2C[C@@H]3[C@H](C2)[C@H]3C(=O)NCC(=O)Nc2cccc(C(F)(F)F)c2)CC1] | 451.5 | 452.3 |
| 46 | 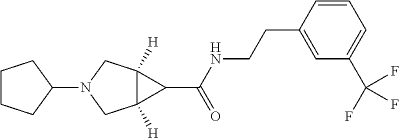 [SMILES: O=C(NCCc1cccc(C(F)(F)F)c1)C1[C@H]2CN(C3CCCC3)C[C@@H]12] | 366.4 | 367.1 |
| 47 | 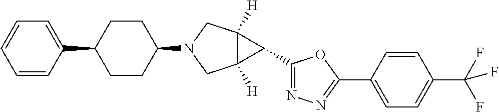 [SMILES: FC(F)(F)c1ccc(-c2nnc([C@H]3[C@@H]4CN([C@H]5CC[C@@H](c6ccccc6)CC5)C[C@@H]43)o2)cc1] | 453.5 | 454.2 |
| 48 | 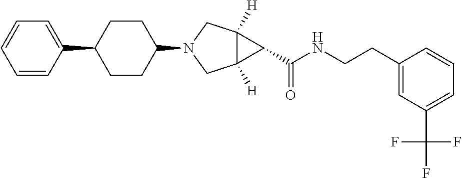 | 456.6 | 457.1 |
| 49 | 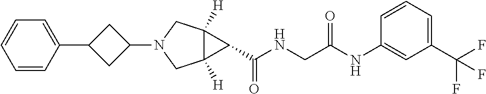 [SMILES: O=C(CNC(=O)[C@H]1[C@@H]2CN(C3CC(c4ccccc4)C3)C[C@@H]21)Nc1cccc(C(F)(F)F)c1] | 457.5 | 458.2 |
| 50 | 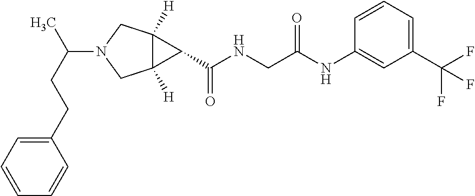 [SMILES: CC(CCc1ccccc1)N1C[C@@H]2[C@H](C1)[C@H]2C(=O)NCC(=O)Nc1cccc(C(F)(F)F)c1] | 459.5 | 460.0 |
| 51 | 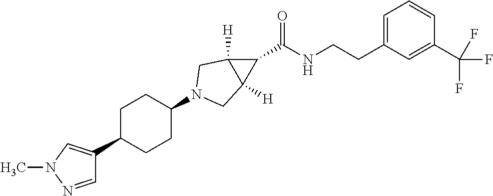 [SMILES: Cn1cc([C@H]2CC[C@@H](N3C[C@@H]4[C@H](C3)[C@H]4C(=O)NCCc3cccc(C(F)(F)F)c3)CC2)cn1] | 460.5 | 461.8 |
| 52 | 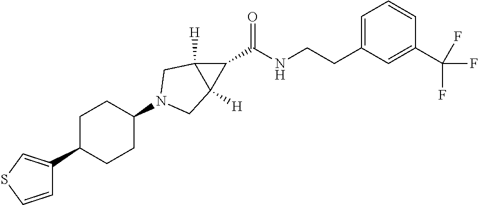 [SMILES: O=C(NCCc1cccc(C(F)(F)F)c1)[C@H]1[C@@H]2CN([C@H]3CC[C@@H](c4ccsc4)CC3)C[C@@H]21] | 462.6 | 463.4 |
| 53 | 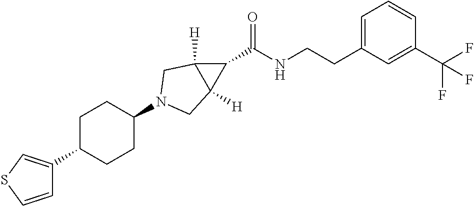 [SMILES: O=C(NCCc1cccc(C(F)(F)F)c1)[C@H]1[C@@H]2CN([C@H]3CC[C@H](c4ccsc4)CC3)C[C@@H]21] | 462.6 | 463.6 |
| 54 | 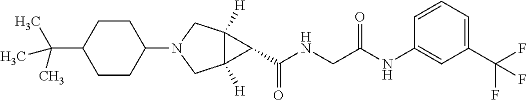 [SMILES: CC(C)(C)C1CCC(N2C[C@@H]3[C@H](C2)[C@H]3C(=O)NCC(=O)Nc2cccc(C(F)(F)F)c2)CC1] | 465.6 | 466.3 |
| 55 | 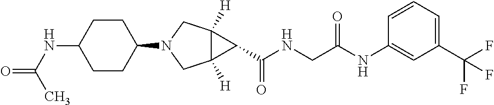 | 466.5 | 467.4 |
| 56 | 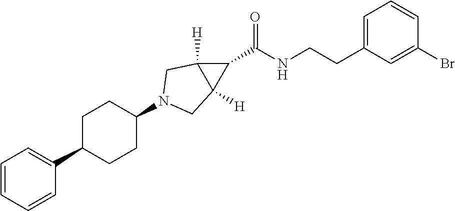 [SMILES: O=C(NCCc1cccc(Br)c1)[C@H]1[C@@H]2CN([C@H]3CC[C@@H](c4ccccc4)CC3)C[C@@H]21] | 467.5 | 467.3 |
| 57 | 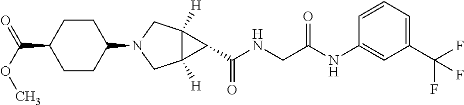 | 467.5 | 468.4 |
| 58 | 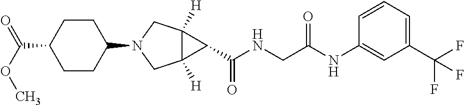 | 467.5 | 468.2 |
| 59 | 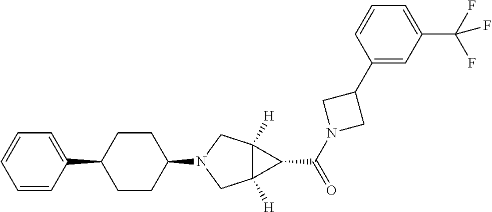 [SMILES: O=C([C@H]1[C@@H]2CN([C@H]3CC[C@@H](c4ccccc4)CC3)C[C@@H]21)N1CC(c2cccc(C(F)(F)F)c2)C1] | 468.6 | 469.2 |
| 60 | 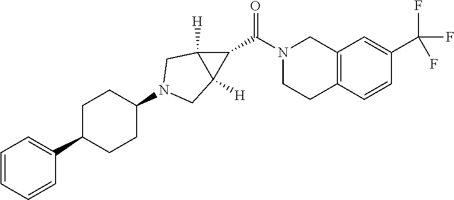 [SMILES: O=C([C@H]1[C@@H]2CN([C@H]3CC[C@@H](c4ccccc4)CC3)C[C@@H]21)N1CCc2ccc(C(F)(F)F)cc2C1] | 468.6 | 469.1 |
| 61 | 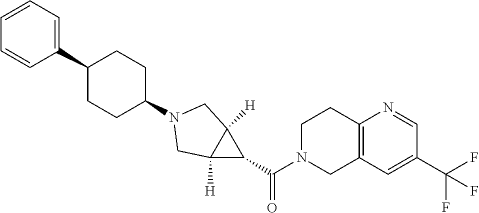 [SMILES: O=C([C@H]1[C@@H]2CN([C@H]3CC[C@@H](c4ccccc4)CC3)C[C@@H]21)N1CCc2ncc(C(F)(F)F)cc2C1] | 469.6 | 470.3 |
| 62 | 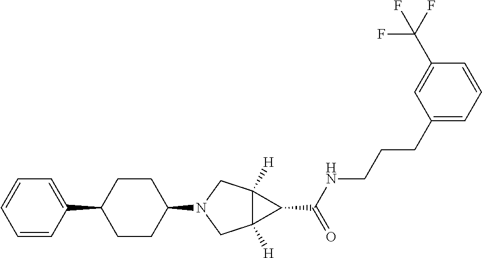 [SMILES: O=C(NCCCc1cccc(C(F)(F)F)c1)[C@H]1[C@@H]2CN([C@H]3CC[C@@H](c4ccccc4)CC3)C[C@@H]21] | 470.6 | 471.3 |
| 63 | 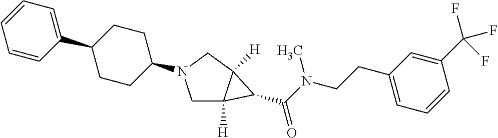 [SMILES: CN(CCc1cccc(C(F)(F)F)c1)C(=O)[C@H]1[C@@H]2CN([C@H]3CC[C@@H](c4ccccc4)CC3)C[C@@H]21] | 470.6 | 471.4 |
| 64 | 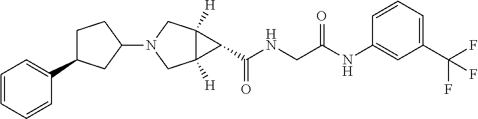 [SMILES: O=C(CNC(=O)[C@H]1[C@@H]2CN(C3CC[C@H](c4ccccc4)C3)C[C@@H]21)Nc1cccc(C(F)(F)F)c1] | 471.5 | 472.0 |
| 65 | 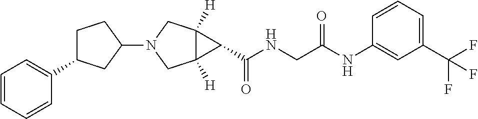 [SMILES: O=C(CNC(=O)[C@H]1[C@@H]2CN(C3CC[C@@H](c4ccccc4)C3)C[C@@H]21)Nc1cccc(C(F)(F)F)c1] | 471.5 | 472.2 |
| 66 | 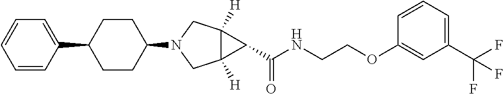 [SMILES: O=C(NCCOc1cccc(C(F)(F)F)c1)[C@H]1[C@@H]2CN([C@H]3CC[C@@H](c4ccccc4)CC3)C[C@@H]21] | 472.6 | 473.5 |
| 67 | 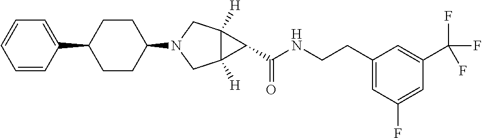 [SMILES: O=C(NCCc1cc(F)cc(C(F)(F)F)c1)[C@H]1[C@@H]2CN([C@H]3CC[C@@H](c4ccccc4)CC3)C[C@@H]21] | 474.5 | 475.2 |
| 68 | 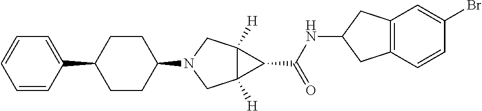 [SMILES: O=C(NC1Cc2ccc(Br)cc2C1)[C@H]1[C@@H]2CN([C@H]3CC[C@@H](c4ccccc4)CC3)C[C@@H]21] | 479.5 | 479.2 |
| 69 | 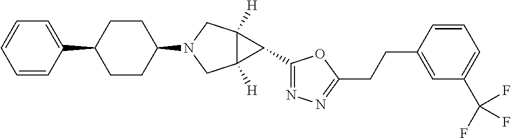 [SMILES: FC(F)(F)c1cccc(CCc2nnc([C@H]3[C@@H]4CN([C@H]5CC[C@@H](c6ccccc6)CC5)C[C@@H]43)o2)c1] | 481.6 | 482.4 |
| 70 | 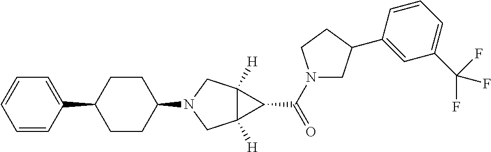 [SMILES: O=C([C@H]1[C@@H]2CN([C@H]3CC[C@@H](c4ccccc4)CC3)C[C@@H]21)N1CCC(c2cccc(C(F)(F)F)c2)C1] | 482.6 | 483.8 |
| 71 | 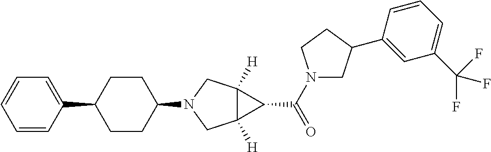 [SMILES: O=C([C@H]1[C@@H]2CN([C@H]3CC[C@@H](c4ccccc4)CC3)C[C@@H]21)N1CCC(c2cccc(C(F)(F)F)c2)C1] | 482.6 | 483.4 |
| 72 | 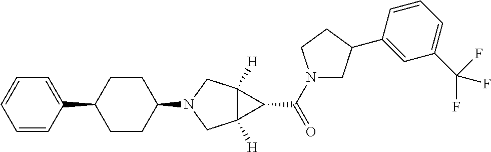 [SMILES: O=C([C@H]1[C@@H]2CN([C@H]3CC[C@@H](c4ccccc4)CC3)C[C@@H]21)N1CCC(c2cccc(C(F)(F)F)c2)C1] | 482.6 | 483.8 |
| 73 | 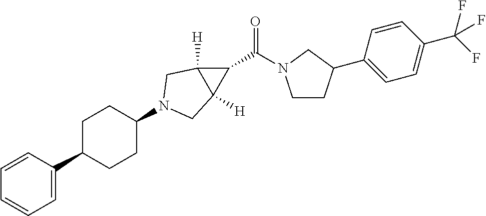 [SMILES: O=C([C@H]1[C@@H]2CN([C@H]3CC[C@@H](c4ccccc4)CC3)C[C@@H]21)N1CCC(c2ccc(C(F)(F)F)cc2)C1] | 482.6 | 483.3 |
| 74 | 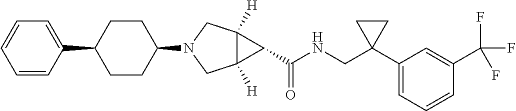 [SMILES: O=C(NCC1(c2cccc(C(F)(F)F)c2)CC1)[C@H]1[C@@H]2CN([C@H]3CC[C@@H](c4ccccc4)CC3)C[C@@H]21] | 482.6 | 483.4 |
| 76 | 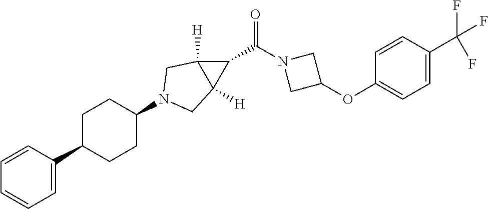 [SMILES: O=C([C@H]1[C@@H]2CN([C@H]3CC[C@@H](c4ccccc4)CC3)C[C@@H]21)N1CC(Oc2ccc(C(F)(F)F)cc2)C1] | 484.6 | 485.2 |
| 77 | 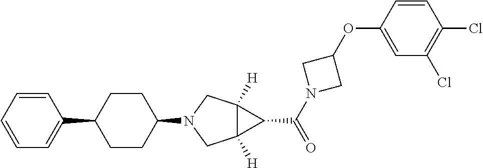 [SMILES: O=C([C@H]1[C@@H]2CN([C@H]3CC[C@@H](c4ccccc4)CC3)C[C@@H]21)N1CC(Oc2ccc(Cl)c(Cl)c2)C1] | 485.5 | 485.2 |
| 78 | 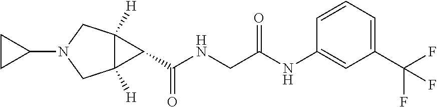 [SMILES: O=C(CNC(=O)[C@H]1[C@@H]2CN(C3CC3)C[C@@H]21)Nc1cccc(C(F)(F)F)c1] | 367.4 | 366.4 |
| 79 | 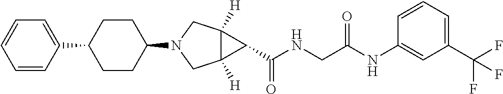 [SMILES: O=C(CNC(=O)[C@H]1[C@@H]2CN([C@H]3CC[C@H](c4ccccc4)CC3)C[C@@H]21)Nc1cccc(C(F)(F)F)c1] | 485.6 | 486.4 |
| 80 | 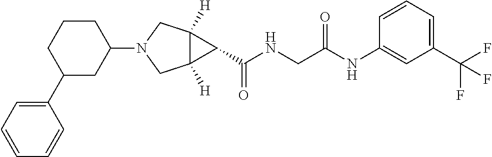 [SMILES: O=C(CNC(=O)[C@H]1[C@@H]2CN(C3CCCC(c4ccccc4)C3)C[C@@H]21)Nc1cccc(C(F)(F)F)c1] | 485.6 | 486.8 |
| 81 | 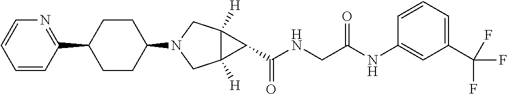 [SMILES: O=C(CNC(=O)[C@H]1[C@@H]2CN([C@H]3CC[C@@H](c4ccccn4)CC3)C[C@@H]21)Nc1cccc(C(F)(F)F)c1] | 486.5 | 487.3 |
| 82 | 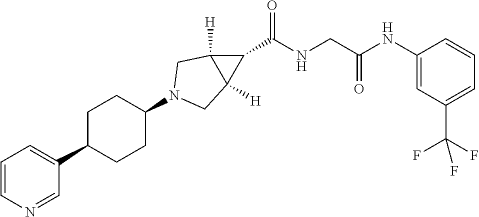 [SMILES: O=C(CNC(=O)[C@H]1[C@@H]2CN([C@H]3CC[C@@H](c4cccnc4)CC3)C[C@@H]21)Nc1cccc(C(F)(F)F)c1] | 486.5 | 487.4 |
| 83 | 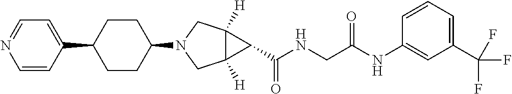 [SMILES: O=C(CNC(=O)[C@H]1[C@@H]2CN([C@H]3CC[C@@H](c4ccncc4)CC3)C[C@@H]21)Nc1cccc(C(F)(F)F)c1] | 486.5 | 487.4 |
| 84 | 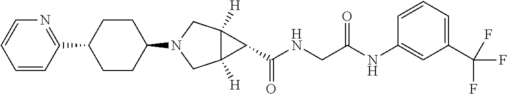 [SMILES: O=C(CNC(=O)[C@H]1[C@@H]2CN([C@H]3CC[C@H](c4ccccn4)CC3)C[C@@H]21)Nc1cccc(C(F)(F)F)c1] | 486.5 | 487.3 |
| 85 | 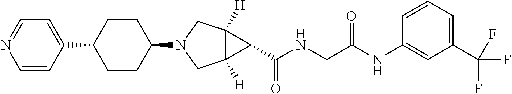 [SMILES: O=C(CNC(=O)[C@H]1[C@@H]2CN([C@H]3CC[C@H](c4ccncc4)CC3)C[C@@H]21)Nc1cccc(C(F)(F)F)c1] | 486.5 | 487.4 |
| 86 | 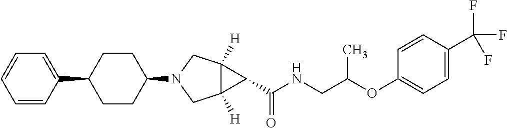 [SMILES: CC(CNC(=O)[C@H]1[C@@H]2CN([C@H]3CC[C@@H](c4ccccc4)CC3)C[C@@H]21)Oc1ccc(C(F)(F)F)cc1] | 486.6 | 487.7 |
| 87 | 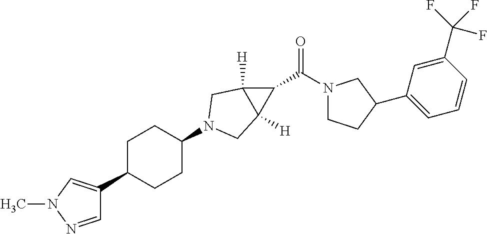 [SMILES: Cn1cc([C@H]2CC[C@@H](N3C[C@@H]4[C@H](C3)[C@H]4C(=O)N3CCC(c4cccc(C(F)(F)F)c4)C3)CC2)cn1] | 486.6 | 487.5 |
| 88 | 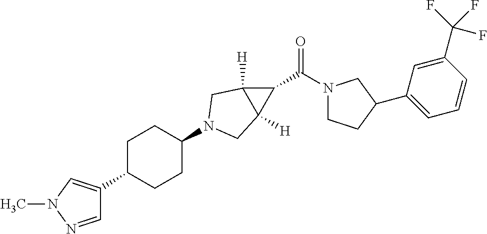 [SMILES: Cn1cc([C@H]2CC[C@H](N3C[C@@H]4[C@H](C3)[C@H]4C(=O)N3CCC(c4cccc(C(F)(F)F)c4)C3)CC2)cn1] | 486.6 | 487.5 |
| 89 | 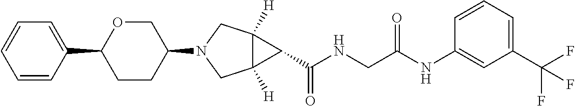 [SMILES: O=C(CNC(=O)[C@H]1[C@@H]2CN([C@H]3CC[C@@H](c4ccccc4)OC3)C[C@@H]21)Nc1cccc(C(F)(F)F)c1] | 487.5 | 488.8 |
| 90 | 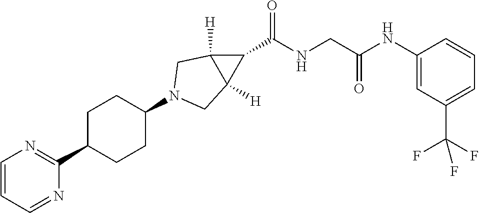 [SMILES: O=C(CNC(=O)[C@H]1[C@@H]2CN([C@H]3CC[C@@H](c4ncccn4)CC3)C[C@@H]21)Nc1cccc(C(F)(F)F)c1] | 487.5 | 488.3 |
| 91 | 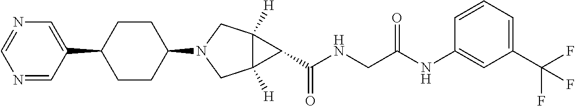 [SMILES: O=C(CNC(=O)[C@H]1[C@@H]2CN([C@H]3CC[C@@H](c4cncnc4)CC3)C[C@@H]21)Nc1cccc(C(F)(F)F)c1] | 487.5 | 488.3 |
| 92 | 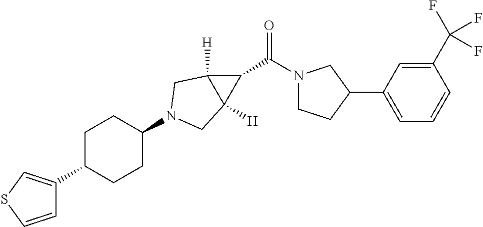 [SMILES: O=C([C@H]1[C@@H]2CN([C@H]3CC[C@H](c4ccsc4)CC3)C[C@@H]21)N1CCC(c2cccc(C(F)(F)F)c2)C1] | 488.6 | 489.9 |
| 93 | 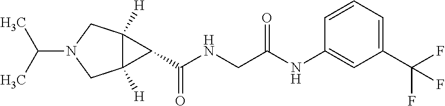 [SMILES: CC(C)N1C[C@@H]2[C@H](C1)[C@H]2C(=O)NCC(=O)Nc1cccc(C(F)(F)F)c1] | 369.4 | 370.2 |
| 94 | 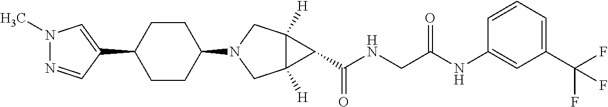 [SMILES: Cn1cc([C@H]2CC[C@@H](N3C[C@@H]4[C@H](C3)[C@H]4C(=O)NCC(=O)Nc3cccc(C(F)(F)F)c3)CC2)cn1] | 489.5 | 490.2 |
| 95 | 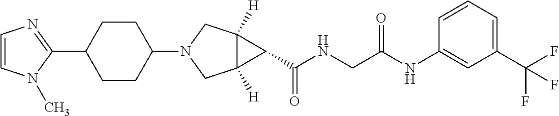 [SMILES: Cn1ccnc1C1CCC(N2C[C@@H]3[C@H](C2)[C@H]3C(=O)NCC(=O)Nc2cccc(C(F)(F)F)c2)CC1] | 489.5 | 490.2 |
| 96 | 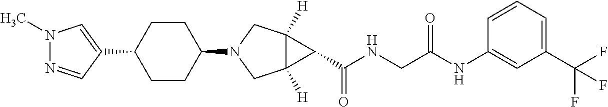 [SMILES: Cn1cc([C@H]2CC[C@H](N3C[C@@H]4[C@H](C3)[C@H]4C(=O)NCC(=O)Nc3cccc(C(F)(F)F)c3)CC2)cn1] | 489.5 | 490.2 |
| 97 | 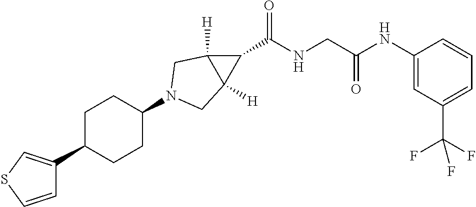 [SMILES: O=C(CNC(=O)[C@H]1[C@@H]2CN([C@H]3CC[C@@H](c4ccsc4)CC3)C[C@@H]21)Nc1cccc(C(F)(F)F)c1] | 491.6 | 492.3 |
| 98 | 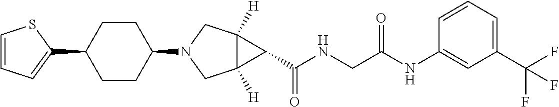 [SMILES: O=C(CNC(=O)[C@H]1[C@@H]2CN([C@H]3CC[C@@H](c4cccs4)CC3)C[C@@H]21)Nc1cccc(C(F)(F)F)c1] | 491.6 | 492.3 |
| 99 | 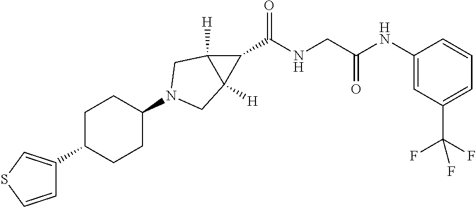 [SMILES: O=C(CNC(=O)[C@H]1[C@@H]2CN([C@H]3CC[C@H](c4ccsc4)CC3)C[C@@H]21)Nc1cccc(C(F)(F)F)c1] | 491.6 | 492.3 |
| 100 | 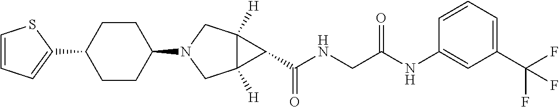 [SMILES: O=C(CNC(=O)[C@H]1[C@@H]2CN([C@H]3CC[C@H](c4cccs4)CC3)C[C@@H]21)Nc1cccc(C(F)(F)F)c1] | 491.6 | 492.2 |
| 101 | 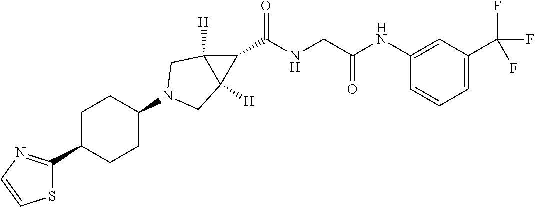 [SMILES: O=C(CNC(=O)[C@H]1[C@@H]2CN([C@H]3CC[C@@H](c4nccs4)CC3)C[C@@H]21)Nc1cccc(C(F)(F)F)c1] | 492.6 | 493.6 |
| 102 | 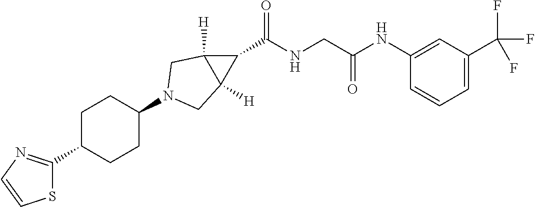 [SMILES: O=C(CNC(=O)[C@H]1[C@@H]2CN([C@H]3CC[C@H](c4nccs4)CC3)C[C@@H]21)Nc1cccc(C(F)(F)F)c1] | 492.6 | 493.5 |
| 103 | 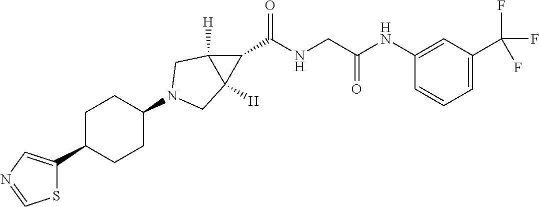 [SMILES: O=C(CNC(=O)[C@H]1[C@@H]2CN([C@H]3CC[C@@H](c4cncs4)CC3)C[C@@H]21)Nc1cccc(C(F)(F)F)c1] | 492.6 | 493.1 |
| 104 | 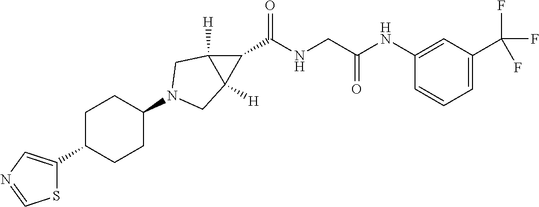 [SMILES: O=C(CNC(=O)[C@H]1[C@@H]2CN([C@H]3CC[C@H](c4cncs4)CC3)C[C@@H]21)Nc1cccc(C(F)(F)F)c1] | 492.6 | 493.3 |
| 105 | 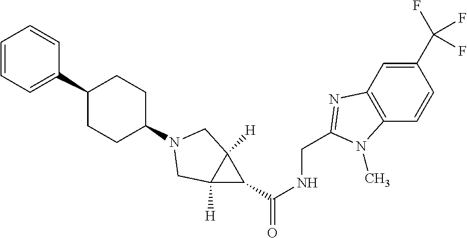 [SMILES: Cn1c(CNC(=O)[C@H]2[C@@H]3CN([C@H]4CC[C@@H](c5ccccc5)CC4)C[C@@H]32)nc2cc(C(F)(F)F)ccc21] | 496.6 | 497.3 |
| 106 | 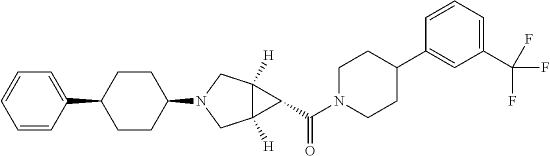 [SMILES: O=C([C@H]1[C@@H]2CN([C@H]3CC[C@@H](c4ccccc4)CC3)C[C@@H]21)N1CCC(c2cccc(C(F)(F)F)c2)CC1] | 496.6 | 497.5 |
| 107 | 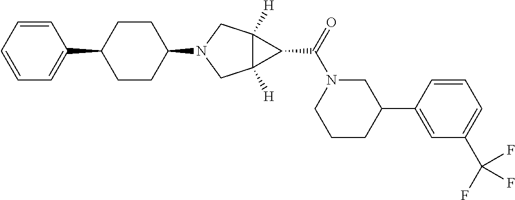 [SMILES: O=C([C@H]1[C@@H]2CN([C@H]3CC[C@@H](c4ccccc4)CC3)C[C@@H]21)N1CCCC(c2cccc(C(F)(F)F)c2)C1] | 496.6 | 497.3 |
| 108 | 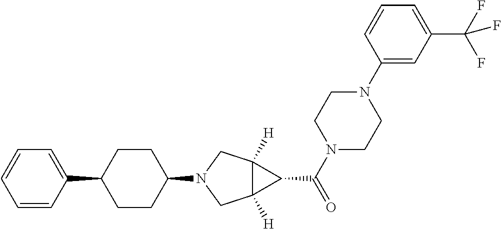 [SMILES: O=C([C@H]1[C@@H]2CN([C@H]3CC[C@@H](c4ccccc4)CC3)C[C@@H]21)N1CCN(c2cccc(C(F)(F)F)c2)CC1] | 497.6 | 498.8 |
| 109 | 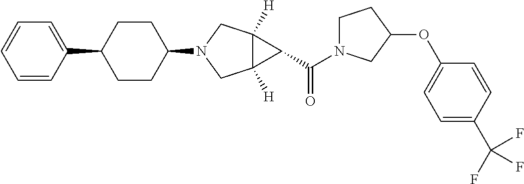 [SMILES: O=C([C@H]1[C@@H]2CN([C@H]3CC[C@@H](c4ccccc4)CC3)C[C@@H]21)N1CCC(Oc2ccc(C(F)(F)F)cc2)C1] | 498.6 | 499.6 |
| 110 | 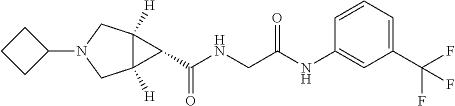 [SMILES: O=C(CNC(=O)[C@H]1[C@@H]2CN(C3CCC3)C[C@@H]21)Nc1cccc(C(F)(F)F)c1] | 381.4 | 382.1 |
| 111 | 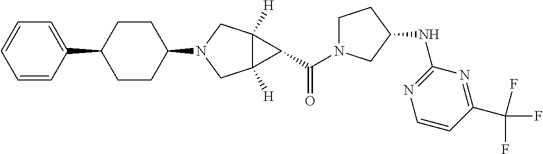 [SMILES: O=C([C@H]1[C@@H]2CN([C@H]3CC[C@@H](c4ccccc4)CC3)C[C@@H]21)N1CC[C@H](Nc2nccc(C(F)(F)F)n2)C1] | 499.6 | 500.6 |
| 112 | 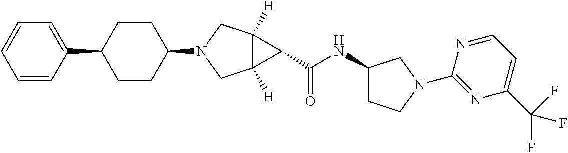 [SMILES: O=C(N[C@@H]1CCN(c2nccc(C(F)(F)F)n2)C1)[C@H]1[C@@H]2CN([C@H]3CC[C@@H](c4ccccc4)CC3)C[C@@H]21] | 499.6 | 500.6 |
| 113 | 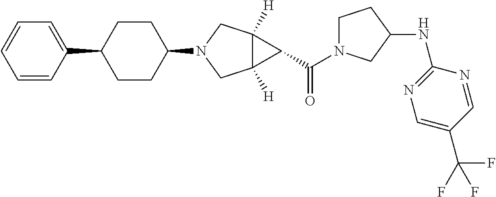 [SMILES: O=C([C@H]1[C@@H]2CN([C@H]3CC[C@@H](c4ccccc4)CC3)C[C@@H]21)N1CCC(Nc2ncc(C(F)(F)F)cn2)C1] | 499.6 | 500.8 |
| 114 | 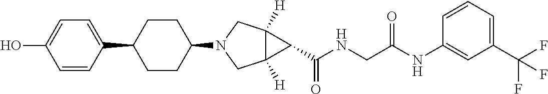 [SMILES: O=C(CNC(=O)[C@H]1[C@@H]2CN([C@H]3CC[C@@H](c4ccc(O)cc4)CC3)C[C@@H]21)Nc1cccc(C(F)(F)F)c1] | 501.6 | 502.4 |
| 115 | 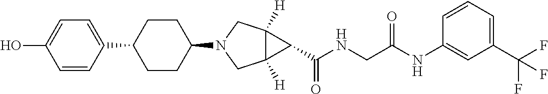 [SMILES: O=C(CNC(=O)[C@H]1[C@@H]2CN([C@H]3CC[C@H](c4ccc(O)cc4)CC3)C[C@@H]21)Nc1cccc(C(F)(F)F)c1] | 501.6 | 502.2 |
| 116 | 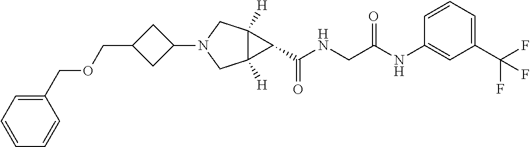 [SMILES: O=C(CNC(=O)[C@H]1[C@@H]2CN(C3CC(COCc4ccccc4)C3)C[C@@H]21)Nc1cccc(C(F)(F)F)c1] | 501.6 | 502.4 |
| 117 | 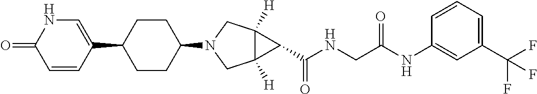 [SMILES: O=C(CNC(=O)[C@H]1[C@@H]2CN([C@H]3CC[C@@H](c4ccc(=O)[nH]c4)CC3)C[C@@H]21)Nc1cccc(C(F)(F)F)c1] | 502.5 | 503.8 |
| 118 | 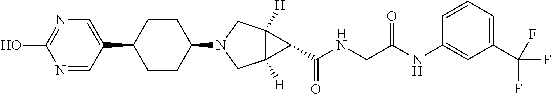 [SMILES: O=C(CNC(=O)[C@H]1[C@@H]2CN([C@H]3CC[C@@H](c4cnc(O)nc4)CC3)C[C@@H]21)Nc1cccc(C(F)(F)F)c1] | 503.5 | 504.2 |
| 119 | 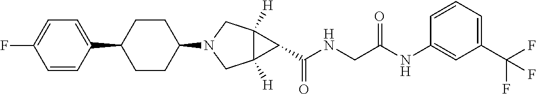 [SMILES: O=C(CNC(=O)[C@H]1[C@@H]2CN([C@H]3CC[C@@H](c4ccc(F)cc4)CC3)C[C@@H]21)Nc1cccc(C(F)(F)F)c1] | 503.5 | 504.3 |
| 120 | 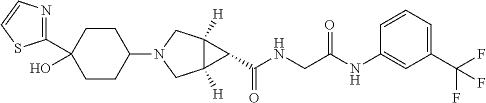 [SMILES: O=C(CNC(=O)[C@H]1[C@@H]2CN(C3CCC(O)(c4nccs4)CC3)C[C@@H]21)Nc1cccc(C(F)(F)F)c1] | 508.6 | 509.4 |
| 121 | 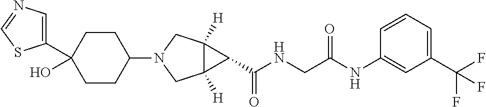 [SMILES: O=C(CNC(=O)[C@H]1[C@@H]2CN(C3CCC(O)(c4cncs4)CC3)C[C@@H]21)Nc1cccc(C(F)(F)F)c1] | 508.6 | 509.4 |
| 122 | 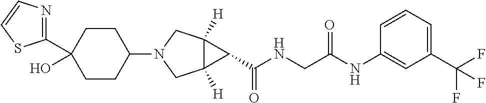 [SMILES: O=C(CNC(=O)[C@H]1[C@@H]2CN(C3CCC(O)(c4nccs4)CC3)C[C@@H]21)Nc1cccc(C(F)(F)F)c1] | 508.6 | 509.4 |
| 123 | 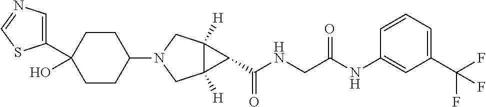 [SMILES: O=C(CNC(=O)[C@H]1[C@@H]2CN(C3CCC(O)(c4cncs4)CC3)C[C@@H]21)Nc1cccc(C(F)(F)F)c1] | 508.6 | 509.4 |
| 124 | 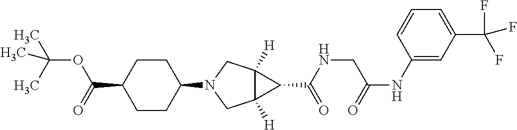 [SMILES: CC(C)(C)OC(=O)[C@H]1CC[C@@H](N2C[C@@H]3[C@H](C2)[C@H]3C(=O)NCC(=O)Nc2cccc(C(F)(F)F)c2)CC1] | 509.6 | 510.2 |
| 125 | 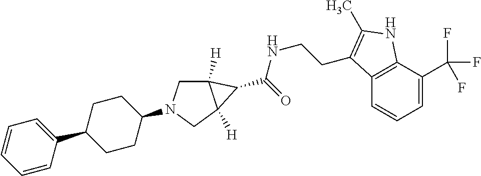 [SMILES: Cc1[nH]c2c(C(F)(F)F)cccc2c1CCNC(=O)[C@H]1[C@@H]2CN([C@H]3CC[C@@H](c4ccccc4)CC3)C[C@@H]21] | 509.6 | 510.4 |
| 126 | 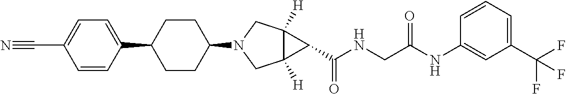 [SMILES: N#Cc1ccc([C@H]2CC[C@@H](N3C[C@@H]4[C@H](C3)[C@H]4C(=O)NCC(=O)Nc3cccc(C(F)(F)F)c3)CC2)cc1] | 510.6 | 511.2 |
| 127 | 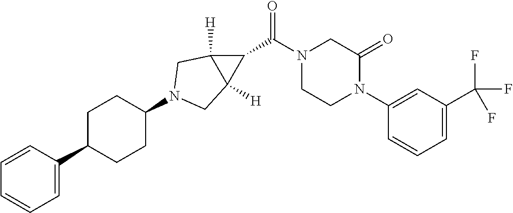 [SMILES: O=C([C@H]1[C@@H]2CN([C@H]3CC[C@@H](c4ccccc4)CC3)C[C@@H]21)N1CCN(c2cccc(C(F)(F)F)c2)C(=O)C1] | 511.6 | 512.2 |
| 128 | 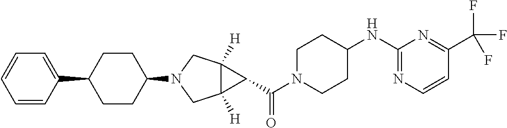 [SMILES: O=C([C@H]1[C@@H]2CN([C@H]3CC[C@@H](c4ccccc4)CC3)C[C@@H]21)N1CCC(Nc2nccc(C(F)(F)F)n2)CC1] | 513.6 | 514.3 |
| 129 | 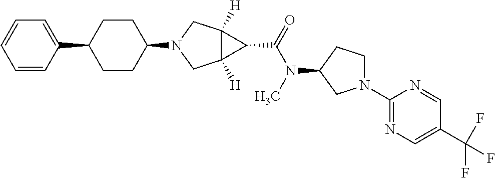 [SMILES: CN(C(=O)[C@H]1[C@@H]2CN([C@H]3CC[C@@H](c4ccccc4)CC3)C[C@@H]21)[C@H]1CCN(c2ncc(C(F)(F)F)cn2)C1] | 513.6 | 514.0 |
| 130 | 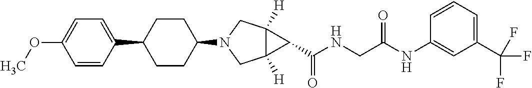 [SMILES: COc1ccc([C@H]2CC[C@@H](N3C[C@@H]4[C@H](C3)[C@H]4C(=O)NCC(=O)Nc3cccc(C(F)(F)F)c3)CC2)cc1] | 515.6 | 516.4 |
| 131 | 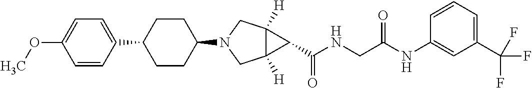 [SMILES: COc1ccc([C@H]2CC[C@H](N3C[C@@H]4[C@H](C3)[C@H]4C(=O)NCC(=O)Nc3cccc(C(F)(F)F)c3)CC2)cc1] | 515.6 | 516.3 |
| 132 | 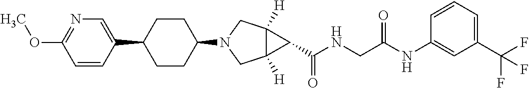 [SMILES: COc1ccc([C@H]2CC[C@@H](N3C[C@@H]4[C@H](C3)[C@H]4C(=O)NCC(=O)Nc3cccc(C(F)(F)F)c3)CC2)cn1] | 516.6 | 517.3 |
| 133 | 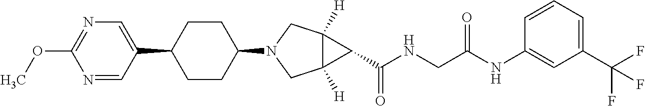 [SMILES: COc1ncc([C@H]2CC[C@@H](N3C[C@@H]4[C@H](C3)[C@H]4C(=O)NCC(=O)Nc3cccc(C(F)(F)F)c3)CC2)cn1] | 517.6 | 518.4 |
| 134 | 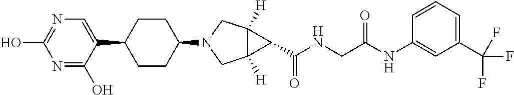 [SMILES: O=C(CNC(=O)[C@H]1[C@@H]2CN([C@H]3CC[C@@H](c4cnc(O)nc4O)CC3)C[C@@H]21)Nc1cccc(C(F)(F)F)c1] | 519.5 | 520.5 |
| 135 | 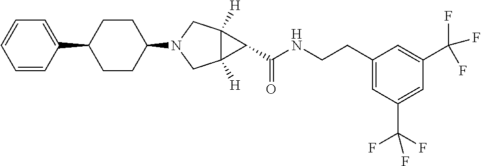 [SMILES: O=C(NCCc1cc(C(F)(F)F)cc(C(F)(F)F)c1)[C@H]1[C@@H]2CN([C@H]3CC[C@@H](c4ccccc4)CC3)C[C@@H]21] | 524.6 | 525.2 |
| 136 | 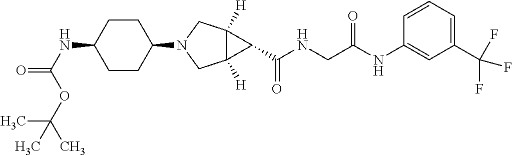 [SMILES: CC(C)(C)OC(=O)N[C@H]1CC[C@@H](N2C[C@@H]3[C@H](C2)[C@H]3C(=O)NCC(=O)Nc2cccc(C(F)(F)F)c2)CC1] | 524.6 | 525.6 |
| 137 | 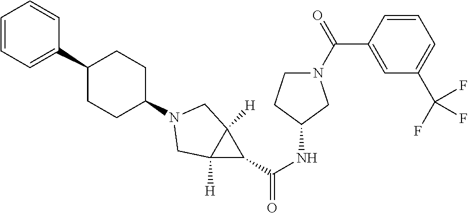 [SMILES: O=C(N[C@@H]1CCN(C(=O)c2cccc(C(F)(F)F)c2)C1)[C@H]1[C@@H]2CN([C@H]3CC[C@@H](c4ccccc4)CC3)C[C@@H]21] | 525.6 | 526.2 |
| 138 | 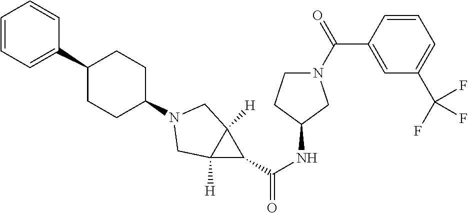 [SMILES: O=C(N[C@H]1CCN(C(=O)c2cccc(C(F)(F)F)c2)C1)[C@H]1[C@@H]2CN([C@H]3CC[C@@H](c4ccccc4)CC3)C[C@@H]21] | 525.6 | 526.3 |
| 139 | 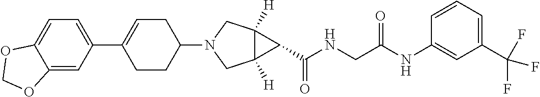 [SMILES: O=C(CNC(=O)[C@H]1[C@@H]2CN(C3CC=C(c4ccc5c(c4)OCO5)CC3)C[C@@H]21)Nc1cccc(C(F)(F)F)c1] | 527.5 | 528.5 |
| 140 | 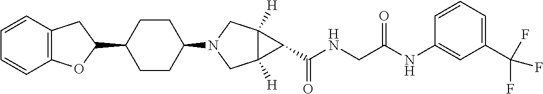 [SMILES: O=C(CNC(=O)[C@H]1[C@@H]2CN([C@H]3CC[C@@H](C4Cc5ccccc5O4)CC3)C[C@@H]21)Nc1cccc(C(F)(F)F)c1] | 527.6 | 528.6 |
| 141 | 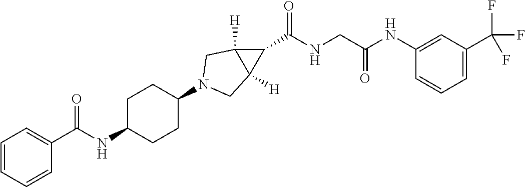 [SMILES: O=C(CNC(=O)[C@H]1[C@@H]2CN([C@H]3CC[C@@H](NC(=O)c4ccccc4)CC3)C[C@@H]21)Nc1cccc(C(F)(F)F)c1] | 528.6 | 529.3 |
| 142 | 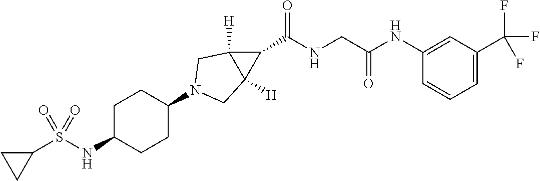 [SMILES: O=C(CNC(=O)[C@H]1[C@@H]2CN([C@H]3CC[C@@H](NS(=O)(=O)C4CC4)CC3)C[C@@H]21)Nc1cccc(C(F)(F)F)c1] | 528.6 | 529.4 |
| 143 | 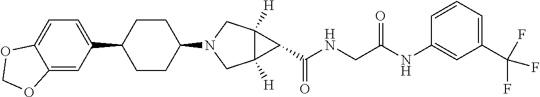 [SMILES: O=C(CNC(=O)[C@H]1[C@@H]2CN([C@H]3CC[C@@H](c4ccc5c(c4)OCO5)CC3)C[C@@H]21)Nc1cccc(C(F)(F)F)c1] | 529.6 | 530.3 |
| 144 | 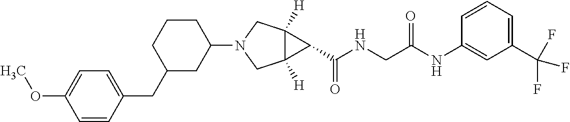 [SMILES: COc1ccc(CC2CCCC(N3C[C@@H]4[C@H](C3)[C@H]4C(=O)NCC(=O)Nc3cccc(C(F)(F)F)c3)C2)cc1] | 529.6 | 530.2 |
| 145 | 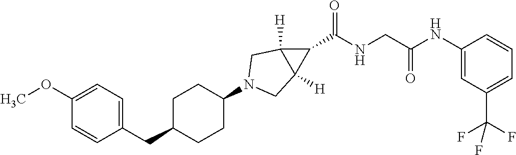 [SMILES: COc1ccc(C[C@H]2CC[C@@H](N3C[C@@H]4[C@H](C3)[C@H]4C(=O)NCC(=O)Nc3cccc(C(F)(F)F)c3)CC2)cc1] | 529.6 | 530.4 |
| 146 | 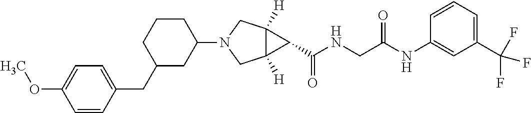 [SMILES: COc1ccc(CC2CCCC(N3C[C@@H]4[C@H](C3)[C@H]4C(=O)NCC(=O)Nc3cccc(C(F)(F)F)c3)C2)cc1] | 529.6 | 530.2 |
| 147 | 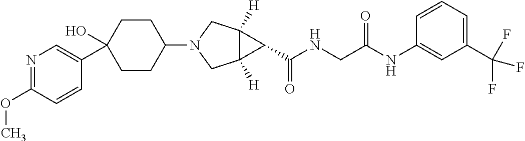 [SMILES: COc1ccc(C2(O)CCC(N3C[C@@H]4[C@H](C3)[C@H]4C(=O)NCC(=O)Nc3cccc(C(F)(F)F)c3)CC2)cn1] | 532.6 | 533.2 |
| 148 | 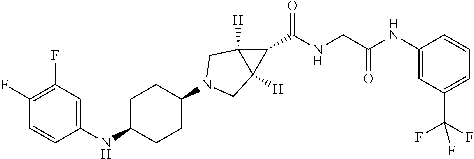 [SMILES: O=C(CNC(=O)[C@H]1[C@@H]2CN([C@H]3CC[C@@H](Nc4ccc(F)c(F)c4)CC3)C[C@@H]21)Nc1cccc(C(F)(F)F)c1] | 536.5 | 537.4 |
| 149 | 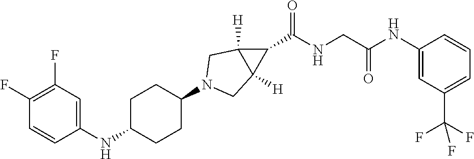 [SMILES: O=C(CNC(=O)[C@H]1[C@@H]2CN([C@H]3CC[C@H](Nc4ccc(F)c(F)c4)CC3)C[C@@H]21)Nc1cccc(C(F)(F)F)c1] | 536.5 | 537.3 |
| 150 | 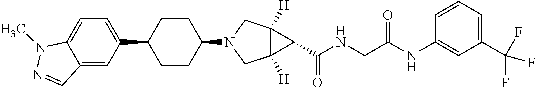 [SMILES: Cn1ncc2cc([C@H]3CC[C@@H](N4C[C@@H]5[C@H](C4)[C@H]5C(=O)NCC(=O)Nc4cccc(C(F)(F)F)c4)CC3)ccc21] | 539.6 | 540.4 |
| 151 | 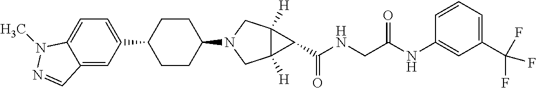 [SMILES: Cn1ncc2cc([C@H]3CC[C@H](N4C[C@@H]5[C@H](C4)[C@H]5C(=O)NCC(=O)Nc4cccc(C(F)(F)F)c4)CC3)ccc21] | 539.6 | 540.4 |
| 152 | 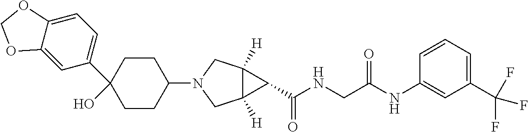 [SMILES: O=C(CNC(=O)[C@H]1[C@@H]2CN(C3CCC(O)(c4ccc5c(c4)OCO5)CC3)C[C@@H]21)Nc1cccc(C(F)(F)F)c1] | 545.6 | 546.3 |
| 153 | 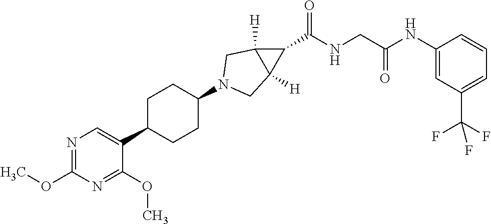 [SMILES: COc1ncc([C@H]2CC[C@@H](N3C[C@@H]4[C@H](C3)[C@H]4C(=O)NCC(=O)Nc3cccc(C(F)(F)F)c3)CC2)c(OC)n1] | 547.6 | 548.4 |
| 154 | 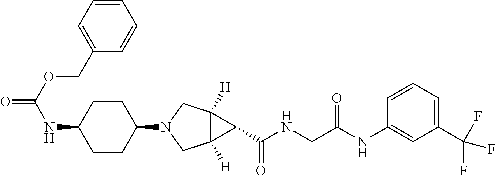 [SMILES: O=C(CNC(=O)[C@H]1[C@@H]2CN([C@H]3CC[C@@H](NC(=O)OCc4ccccc4)CC3)C[C@@H]21)Nc1cccc(C(F)(F)F)c1] | 558.6 | 559.2 |
| 155 | 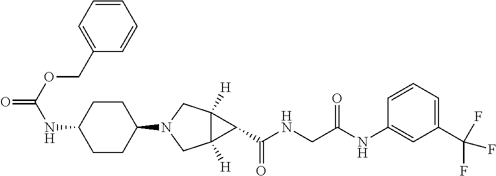 [SMILES: O=C(CNC(=O)[C@H]1[C@@H]2CN([C@H]3CC[C@H](NC(=O)OCc4ccccc4)CC3)C[C@@H]21)Nc1cccc(C(F)(F)F)c1] | 558.6 | 559.4 |
| 156 | 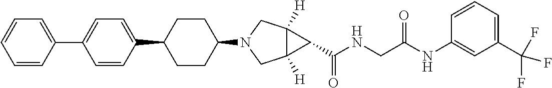 [SMILES: O=C(CNC(=O)[C@H]1[C@@H]2CN([C@H]3CC[C@@H](c4ccc(-c5ccccc5)cc4)CC3)C[C@@H]21)Nc1cccc(C(F)(F)F)c1] | 561.7 | 562.2 |
| 157 | 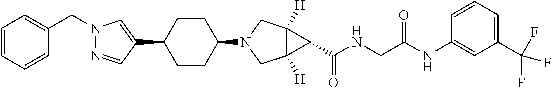 [SMILES: O=C(CNC(=O)[C@H]1[C@@H]2CN([C@H]3CC[C@@H](c4cnn(Cc5ccccc5)c4)CC3)C[C@@H]21)Nc1cccc(C(F)(F)F)c1] | 565.6 | 566.2 |
| 158 | 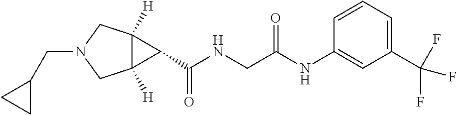 [SMILES: O=C(CNC(=O)[C@H]1[C@@H]2CN(CC3CC3)C[C@@H]21)Nc1cccc(C(F)(F)F)c1] | 381.4 | 382.1 |
| 159 | 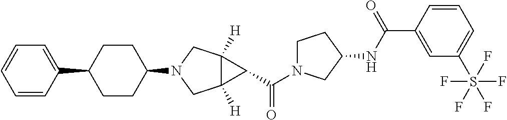 [SMILES: O=C(N[C@H]1CCN(C(=O)[C@H]2[C@@H]3CN([C@H]4CC[C@@H](c5ccccc5)CC4)C[C@@H]32)C1)c1cccc(S(F)(F)(F)(F)F)c1] | 583.7 | 584.1 |
| 160 | 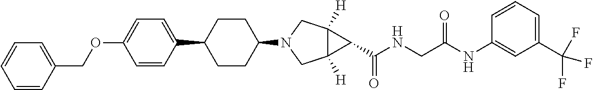 [SMILES: O=C(CNC(=O)[C@H]1[C@@H]2CN([C@H]3CC[C@@H](c4ccc(OCc5ccccc5)cc4)CC3)C[C@@H]21)Nc1cccc(C(F)(F)F)c1] | 591.7 | 592.4 |
| 161 | 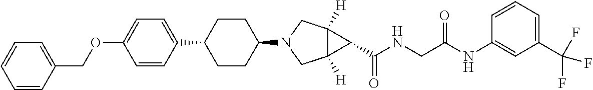 [SMILES: O=C(CNC(=O)[C@H]1[C@@H]2CN([C@H]3CC[C@H](c4ccc(OCc5ccccc5)cc4)CC3)C[C@@H]21)Nc1cccc(C(F)(F)F)c1] | 591.7 | 592.4 |
| 162 | 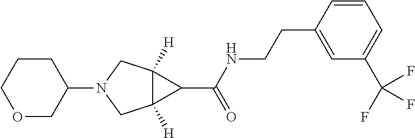 [SMILES: O=C(NCCc1cccc(C(F)(F)F)c1)C1[C@H]2CN(C3CCCOC3)C[C@@H]12] | 382.4 | 383.3 |
| 163 | 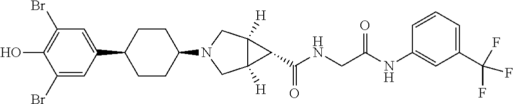 [SMILES: O=C(CNC(=O)[C@H]1[C@@H]2CN([C@H]3CC[C@@H](c4cc(Br)c(O)c(Br)c4)CC3)C[C@@H]21)Nc1cccc(C(F)(F)F)c1] | 659.3 | 660.8 |

### ASSAYS

### CCR2 Membrane Binding Assay

The radio-ligand binding assay was done using scintillation proximity assay (SPA) technology. Briefly, membranes (1 μg per assay point) from Ba/F3 cells transfected with human CCR2, and wheat germ agglutinin-coated SPA beads (80 μg per point; Amersham, Arlington Heights, Ill.), were pre-incubated for 30 min at room temperature in CCR2 buffer (50 mM HEPES (pH=7.4), 10 mM MgCl₂, 10 mM NaCl, 1 mM CaCL₂, 0.1% BSA, 10 μg/ml Saponin). At this point pre-bound SPA bead, membrane complex was incubated at room temperature for 4 h with varying concentrations of punitive CCR2 antagonists (in 1% DMSO, final) and 0.03 nM ¹²⁵I-rhMCP-1 in CCR2Buffer (S.A. 2200 Ci/mmol, PerkinElmer Life and Analytical Science, Boston, Mass.; NEX332). Binding competition was measured using a 1450 Microbeta Trilux counter (Wallac, Gaithersburg, Md.). Binding constants (IC₅₀ and slope) were calculated using GraphPad Prism software (GraphPad Software, Inc., La Jolla Calif.).

Table 2 contains a list of representative compounds which were tested in the above assay. The compounds exhibited 1050 values of less than or equal to 13.6 nM to as low as 1.8 nM. Table 3 contains a list of additional compounds. Compounds with IC50 values less than 50 nM are designated as A class compounds. Compounds with IC50 values between 51 nM and 200 nM are designated as B class compounds. Compounds with IC50 values between 201 nM and 700 nM are designated as C class compounds. Finally, compounds with IC50 values between 701 nM and 110 nM are designated as D class compounds

TABLE 2

| No. | IC50 (nM) |
|---|---|
| 4 | 3.5 |
| 81 | 1.8 |
| 82 | 4.0 |
| 83 | 11.0 |
| 89 | 3.6 |
| 90 | 7.6 |
| 94 | 13.6 |
| 97 | 3.7 |
| 114 | 3.1 |
| 119 | 6.1 |
| 120 | 3.9 |
| 130 | 4.0 |
| 132 | 3.4 |
| 143 | 4.1 |
| 147 | 4.5 |
| 150 | 6.7 |
| 152 | 10.0 |
| 153 | 4.5 |
| 154 | 8.3 |
| 156 | 7.4 |

TABLE 3

| No. | IC50 (nM) |
|---|---|
| 1 | B |
| 2 | B |
| 6 | B |
| 8 | C |
| 9 | B |
| 10 | D |
| 11 | B |
| 12 | C |
| 13 | B |
| 14 | D |
| 15 | C |
| 16 | D |
| 18 | C |
| 19 | C |
| 20 | D |
| 21 | B |
| 22 | B |
| 23 | C |
| 25 | D |
| 27 | B |
| 29 | D |
| 30 | B |
| 31 | B |
| 32 | C |
| 33 | C |
| 34 | C |
| 35 | D |
| 36 | C |
| 37 | D |
| 38 | C |
| 39 | D |
| 40 | B |
| 41 | C |
| 42 | B |
| 44 | C |
| 45 | C |
| 46 | C |
| 48 | A |
| 49 | B |
| 50 | D |
| 54 | D |
| 55 | C |
| 58 | C |
| 59 | C |
| 61 | C |
| 62 | B |
| 64 | B |
| 65 | C |
| 67 | B |
| 68 | B |
| 70 | A |
| 71 | A |
| 57 | A |
| 73 | B |
| 74 | C |
| 76 | D |
| 77 | B |
| 78 | C |
| 79 | D |
| 80 | D |
| 84 | C |
| 85 | C |
| 91 | A |
| 93 | B |
| 95 | B |
| 96 | D |
| 98 | A |
| 99 | B |
| 100 | C |
| 106 | D |
| 108 | C |
| 110 | C |
| 111 | B |
| 112 | C |
| 113 | C |
| 115 | A |
| 116 | C |
| 117 | A |
| 118 | C |
| 121 | A |
| 122 | B |
| 123 | C |
| 124 | C |
| 125 | C |
| 126 | A |
| 127 | D |
| 128 | C |
| 129 | C |
| 131 | D |
| 133 | A |
| 134 | B |
| 135 | C |
| 136 | B |
| 139 | C |
| 140 | C |
| 141 | A |
| 142 | D |
| 144 | B |
| 145 | B |
| 146 | C |
| 148 | A |
| 149 | C |
| 151 | B |
| 155 | C |
| 157 | A |
| 158 | D |
| 160 | A |
| 161 | C |
| 162 | C |
| 163 | A |

1. A compound of Formula I: 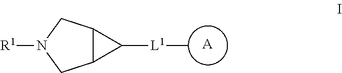
or a pharmaceutically acceptable salt thereof, wherein:
L¹ is selected from the group consisting of:
(a) —C(O)—,
(b) —C(O)-heterocyclyl-,
(c) —C(O)-heterocyclyl-O—,
(d) —C(O)-heterocyclyl-N(R⁴)—,
(e) —C(O)-heterocyclyl-N(R⁴)C(O)—,
(f) —C(O)N(R⁴)—,
(g) —C(O)N(R⁴)—C₁₋₆alkylenyl-,
(h) —C(O)N(R⁴)—C₁₋₆alkylenyl-O—,
(i) —C(O)N(R⁴)—C₁₋₆alkylenyl-C₃₋₈cycloalkyl-,
(j) —C(O)N(R⁴)—C₁₋₆alkylenyl-C(O)—N(R⁴)—,
(k) —C(O)N(R⁴)-heterocyclyl-,
(l) —C(O)N(R⁴)-heterocyclyl-C(O)—, and
(m) 1,3,4-oxadiazolyl,
wherein said heterocyclyl or cycloalkyl groups are unsubstituted or substituted with 1-3 R⁶ groups, and wherein heterocyclyl is a 4-7-membered ring containing 1-2 N heteroatoms;
Ring A is selected from the group consisting of:
(a) aryl,
(b) C₃₋₈cycloalkyl,
(c) C₃₋₈ cycloalkenyl,
(d) 5 or 6-membered heterocyclyl,
(e) 5 or 6-membered heterocyclenyl,
(f) 5 or 6-membered monocyclic heteroaryl,
(g) 9- or 10-membered cycloalkylaryl,
(h) 9- or 10-membered heterocyclylheteraryl,
(i) 9- or 10-membered heterocyclylaryl, and
(j) 9- or 10-membered heteroarylaryl,
wherein said heterocyclyl, heterocyclenyl, or heteroaryl rings have 1-4 hetero-atoms selected from the group consisting of O, N, and S; and wherein said aryl, cycloalkyl, cycloalkenyl, heterocyclyl, heterocyclenyl, heteroaryl, 9- or 10-membered cycloalkylaryl, 9- or 10-membered heterocyclylheteroaryl, 9- or 10-membered heterocyclylaryl, or 9- or 10-membered heteroarylaryl are unsubstituted or substituted with 1-3 R⁵ groups;
R¹ is selected from the group consisting of:
(a) C₁₋₆alkyl,
(b) C₃₋₈cycloalkyl,
(c) C₃₋₈cycloalkenyl,
(d) 5 or 6-membered heterocyclyl, and
(e) 7- or 8-membered bicycloalkyl,
wherein said alkyl, cycloalkyl, cylcoalkenyl, heterocyclyl, and 7- or 8-membered bicycloalkyl groups are unsubstituted or substituted with 1-3 R² groups, and wherein said heterocyclyl, or heteroaryl rings have 1-4 hetero-atoms selected from the group consisting of O, N, and S;
each R² is independently selected from the group consisting of:
(a) hydrogen,
(b) HO—,
(c) halo,
(d) C₁₋₆alkyl,
(e) —C₁₋₆alkyl-OH,
(f) —O—C₁₋₆alkyl,
(g) aryl-C₁₋₆alkyl-O—C₁₋₆alkyl-,
(h) C₂₋₆alkenyl,
(i) oxo,
(j) —NH—C(O)—C₁₋₆alkyl,
(k) —CO₂—C₁₋₆alkyl,
(l) —NH—CO₂—C₁₋₆alkyl,
(m) —NH—C(O)-aryl,
(n) —NH—SO₂—C₃₋₈cycloalkyl,
(o) —NH-aryl,
(p) aryl,
(q) aryl-C₁₋₆alkyl-,
(r) C₃₋₈cycloalkyl,
(s) 5-7-membered heterocyclyl,
(t) 5-7-membered heterocyclenyl,
(u) 5 or 6-membered monocyclic heteroaryl, and
(v) 9-membered heteroarylaryl,
wherein said alkyl, aryl, cycloalkyl, heterocyclyl, heterocyclenyl and heteroaryl groups are unsubstituted or substituted with 1-3 R³ groups, and wherein said heterocyclyl, heterocyclenyl, or heteroaryl rings have 1-4 hetero-atoms selected from the group consisting of O, N and S,
each R³ is independently selected from the group consisting of:
(a) oxo,
(b) HO—,
(c) halo,
(d) C₁₋₆alkyl,
(e) aryl,
(f) aryl-C₁₋₆alkyl-,
(g) C₁₋₆alkoxyl,
(h) aryl-C₁₋₆alkyl-O—, and
(i) cyano;
R⁴ is selected from the group consisting of:
(a) hydrogen,
(b) C₁₋₆alkyl, and
(c) CF₃-phenyl-C₁₋₃alkyl-;
each R⁵ is independently selected from the group consisting of:
(a) oxo,
(b) HO—,
(c) halo,
(d) C₁₋₆alkyl,
(e) C₁₋₆haloalkyl,
(f) aryl,
(g) aryl-C₁₋₆alkyl-,
(h) C₁₋₆alkoxyl,
(i) aryl-C₁₋₆alkyl-O—, and
(j) cyano;
(k) F₅S—;
each R⁶ is independently selected from the group consisting of:
(a) hydrogen,
(b) bromo,
(c) chloro,
(d) fluoro,
(e) methyl,
(f) ethyl,
(g) hydroxyl,
(h) cyano,
(i) oxo,
(j) amino, and
(k) C₁₋₃alkylamino; and
m is 0, 1, 2, or 3.

2. The compound of claim 1 or pharmaceutically acceptable salt thereof, wherein:
L¹ is selected from the group consisting of:

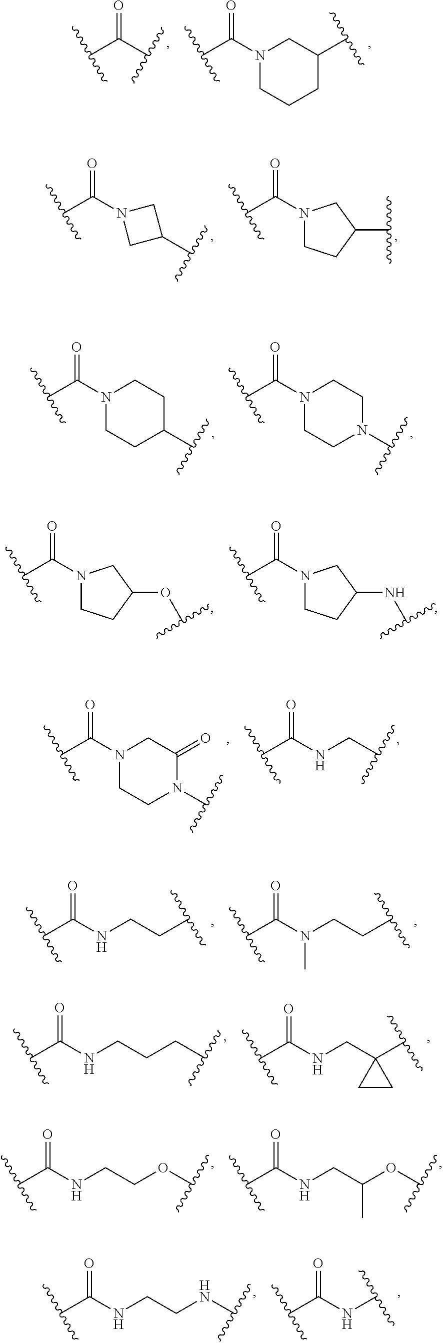

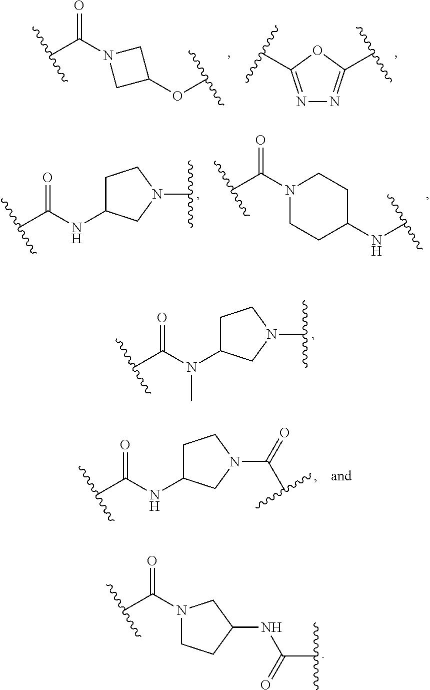

3. The compound of claim 2 or pharmaceutically acceptable salt thereof, wherein:
L¹ is

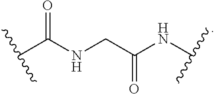 [SMILES: *NC(=O)CNC(*)=O]

4. The compound of claim 1 or pharmaceutically acceptable salt thereof, wherein:
Ring A is selected from the group consisting of: 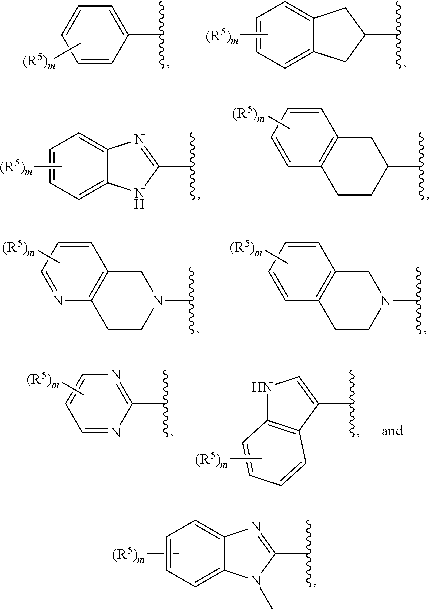
wherein m is 0, 1, 2, or 3.

5. The compound of claim 4 or pharmaceutically acceptable salt thereof, wherein:
Ring A is

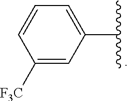 [SMILES: *c1cccc(C(F)(F)F)c1]

6. The compound of claim 1 or pharmaceutically acceptable salt thereof, wherein:
R¹ is selected from the group consisting of the following: 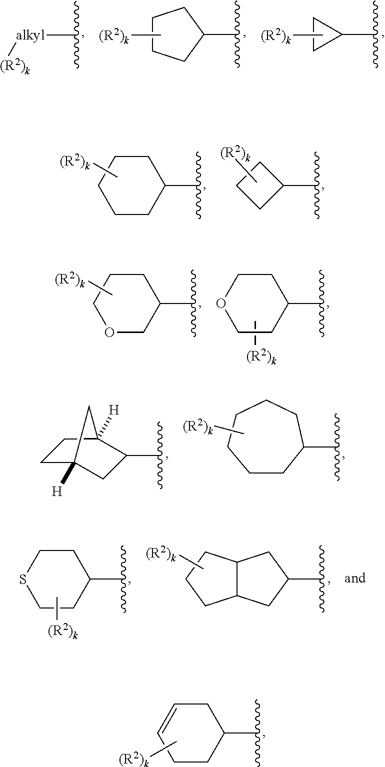
wherein k is 0, 1, 2 or 3.

7. The compound of claim 6 or pharmaceutically acceptable salt thereof, wherein:
Ring A is selected from the group consisting of: 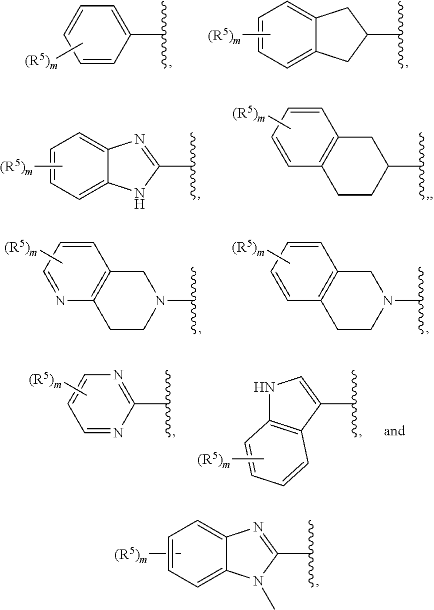
wherein m is 0, 1, 2, or 3.

8. The compound of claim 7 or pharmaceutically acceptable salt thereof, wherein:
L¹ is

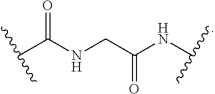 [SMILES: *NC(=O)CNC(*)=O]

9. A compound which is selected from the group consisting of: 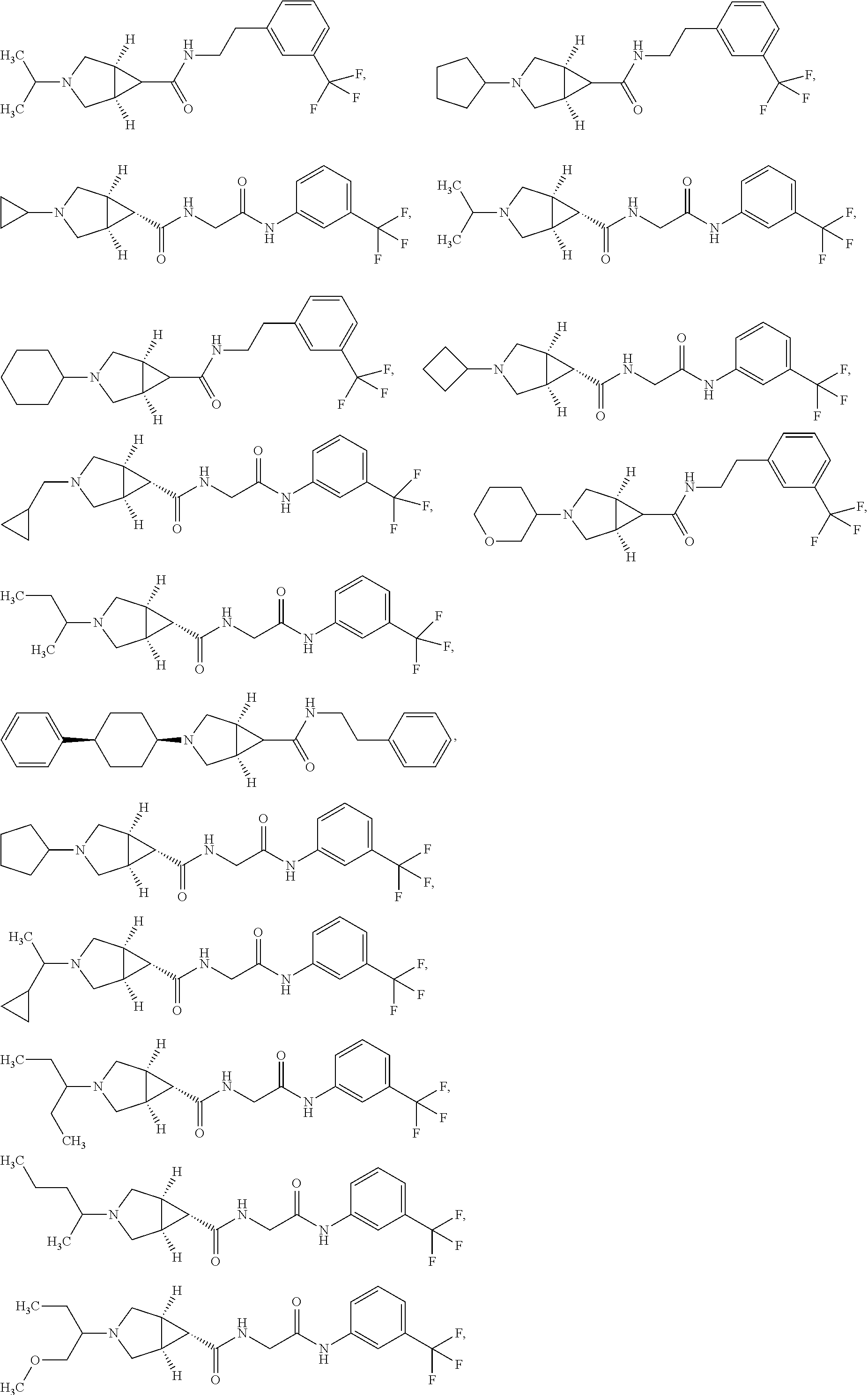 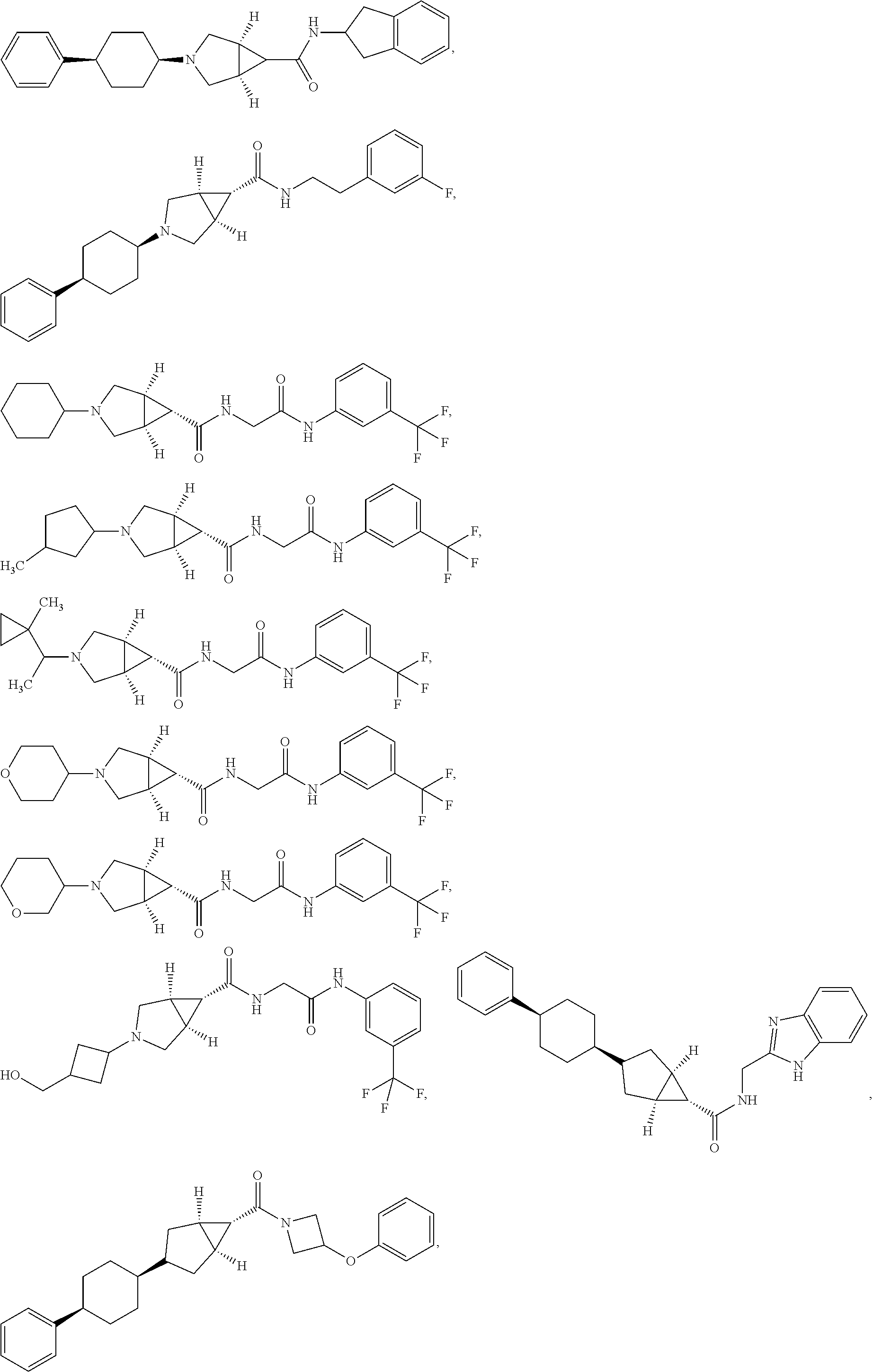 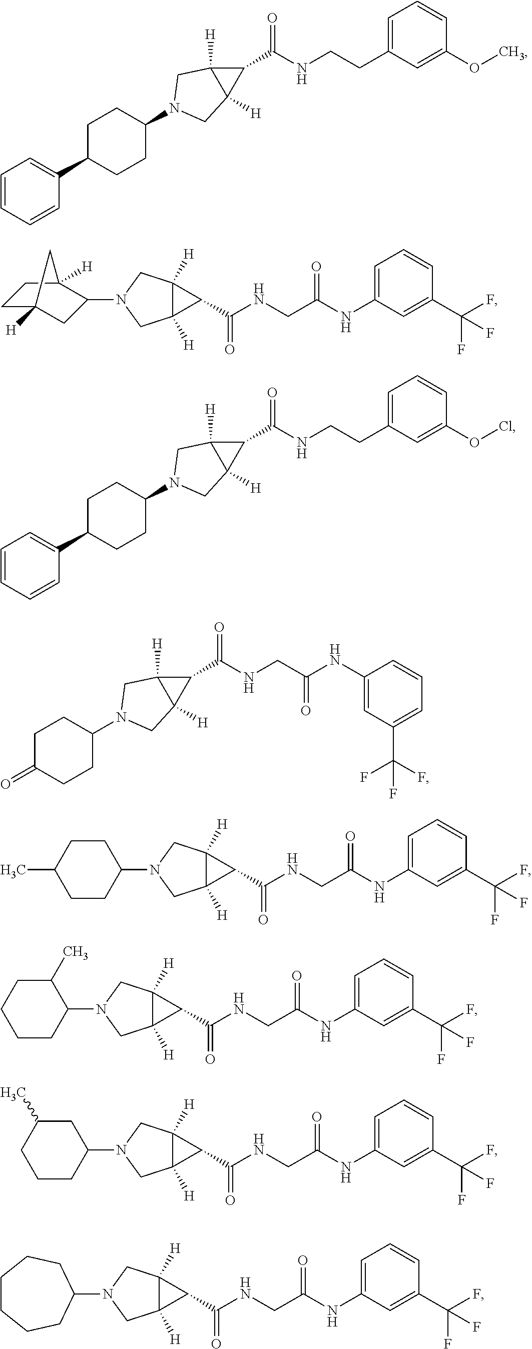 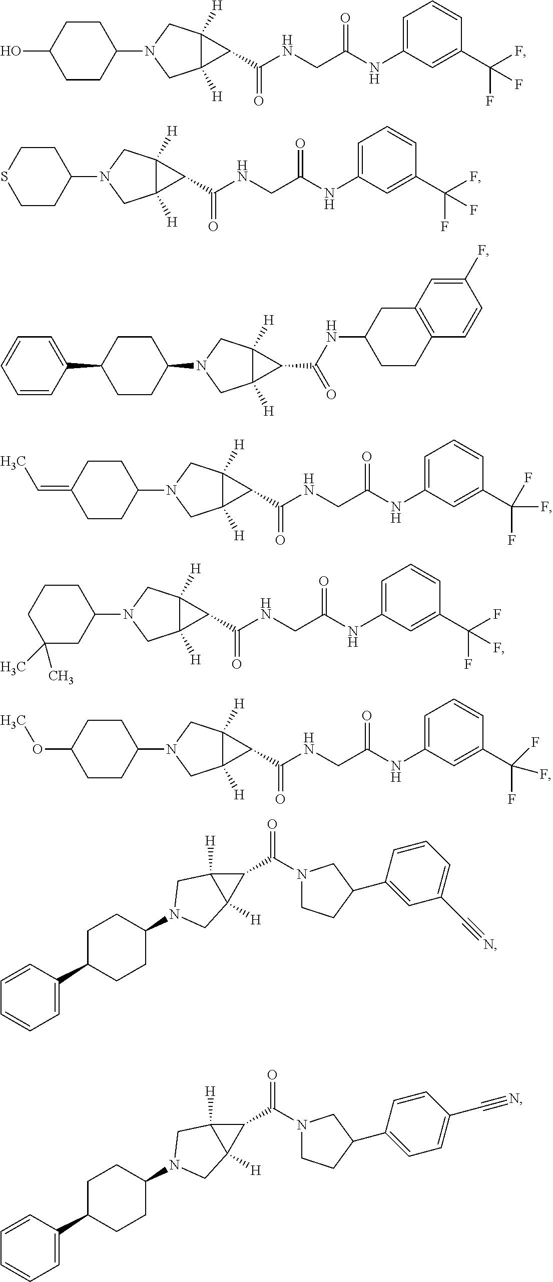 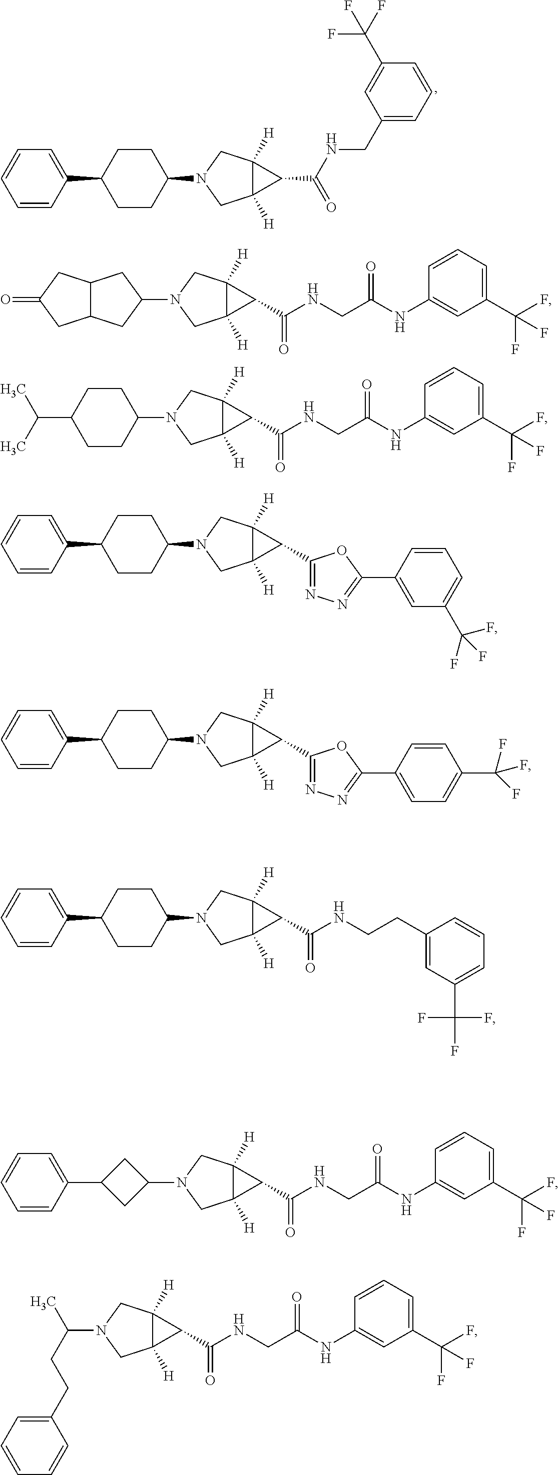 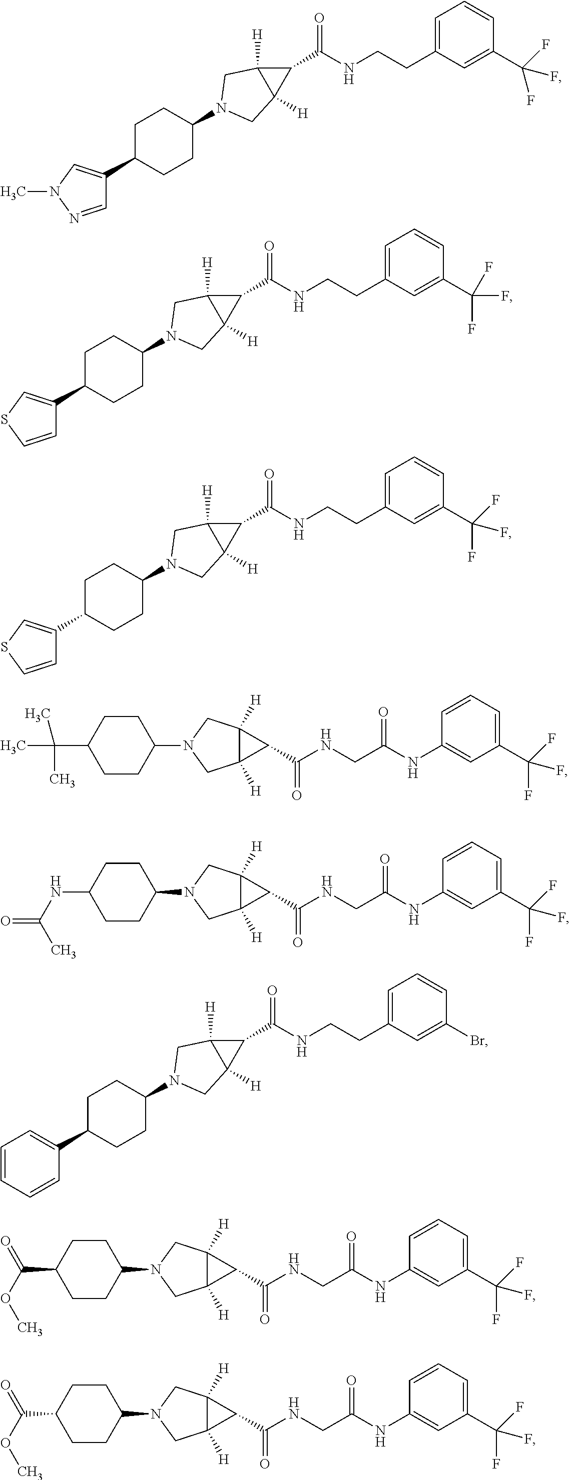 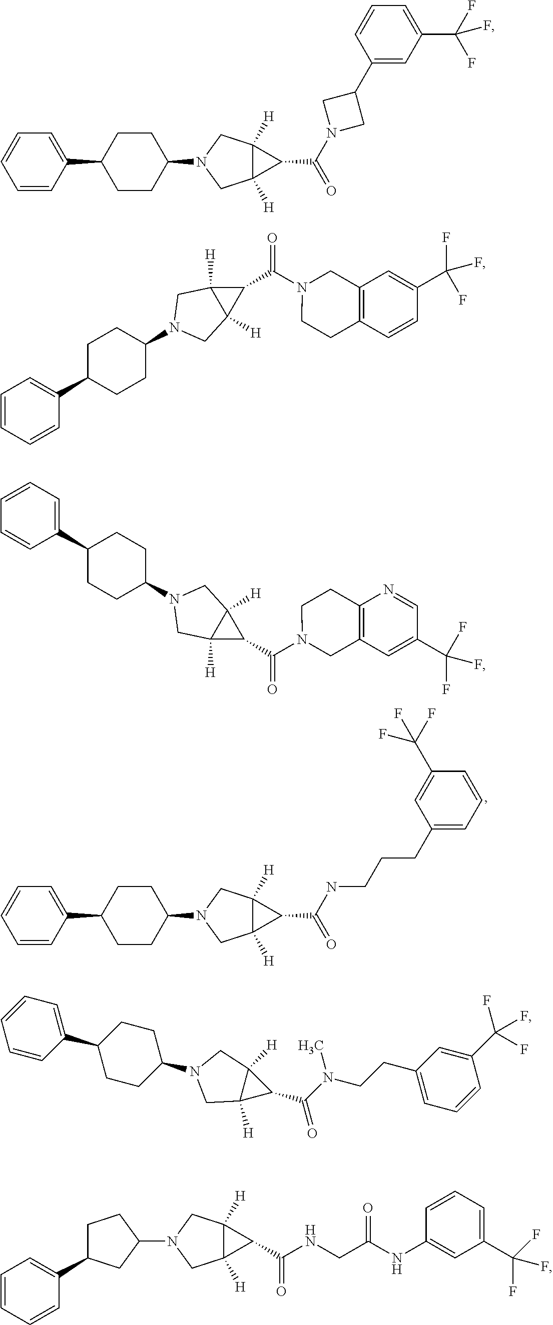 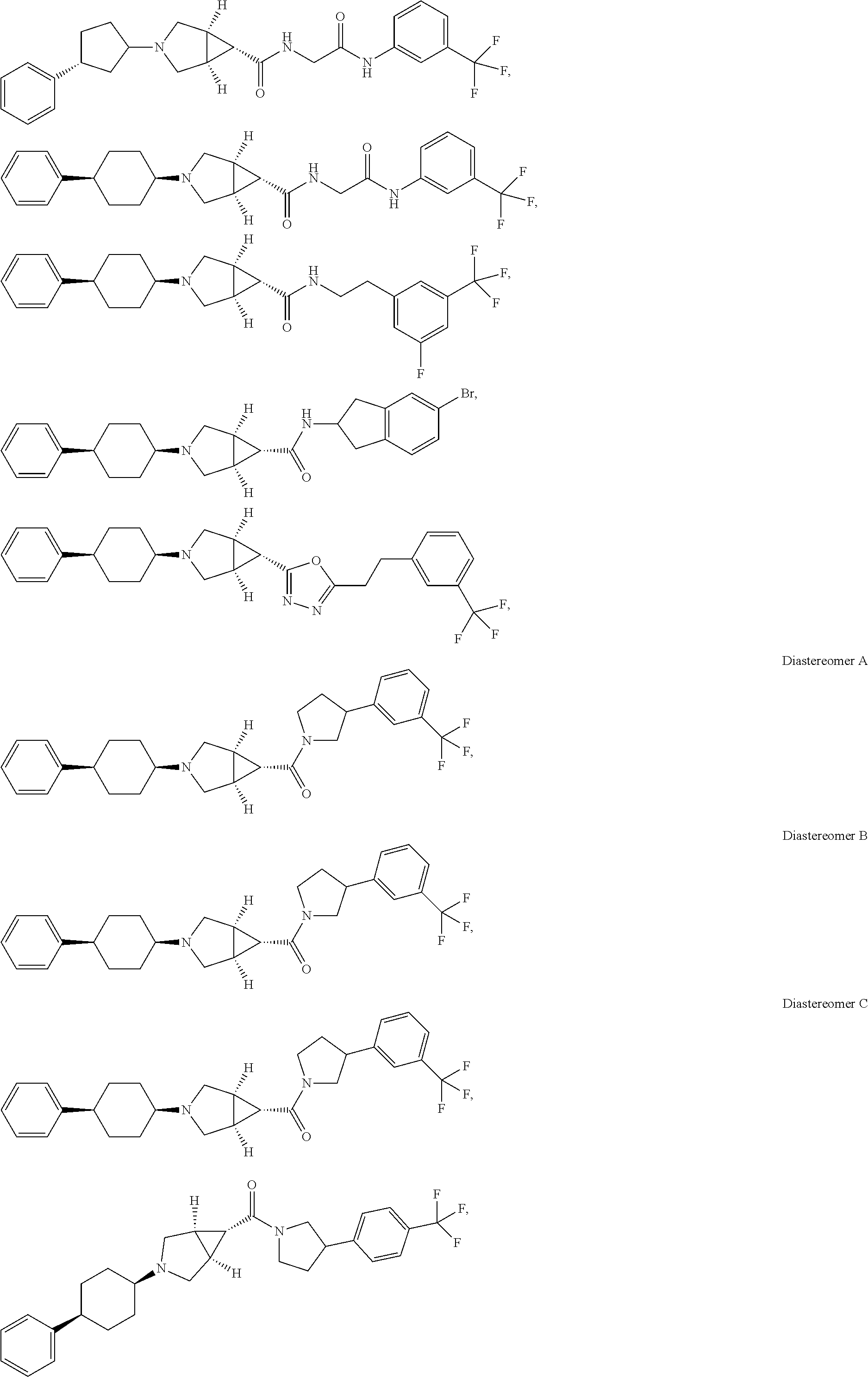 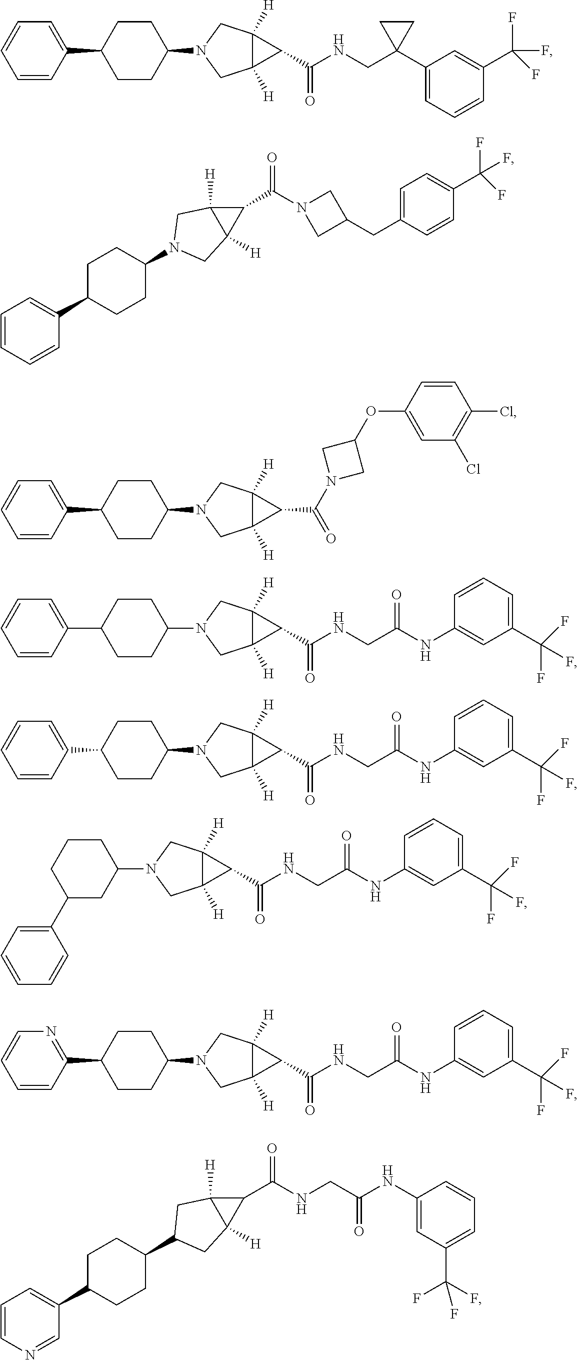 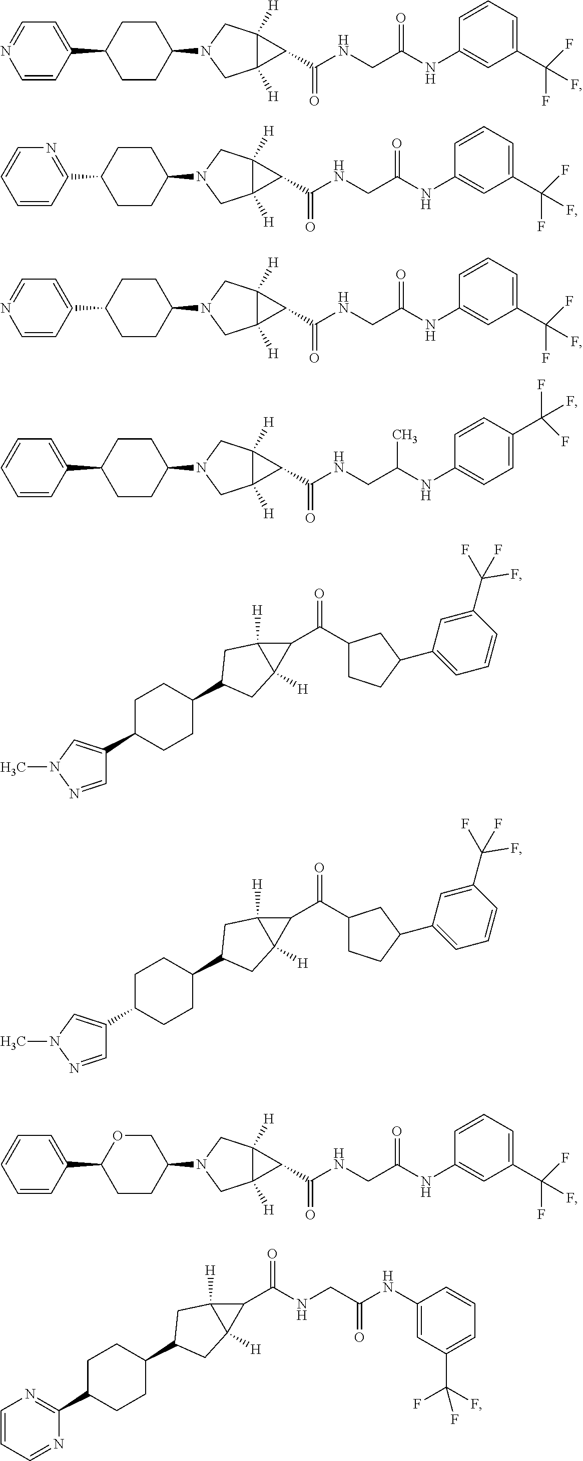 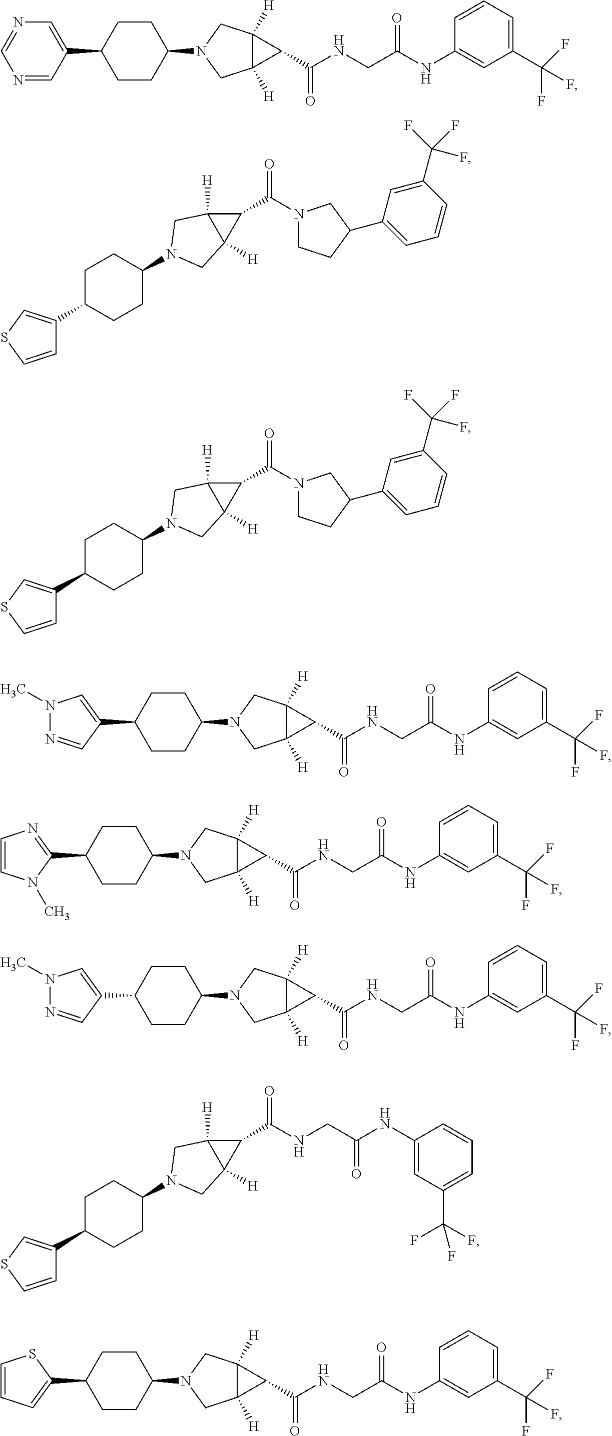 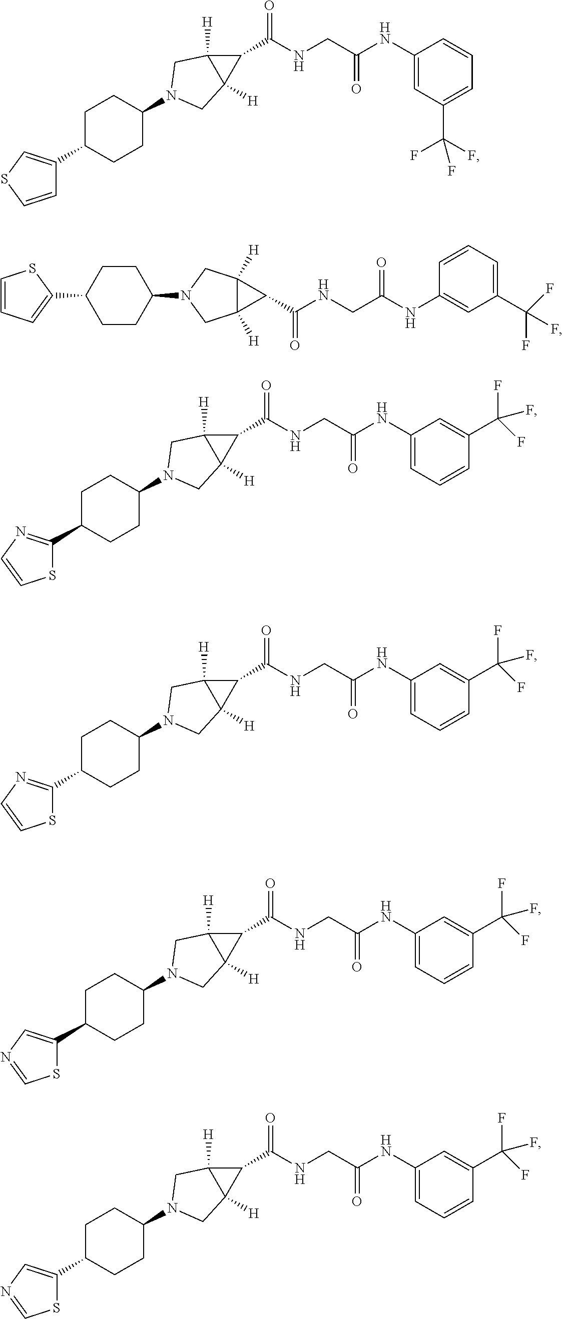 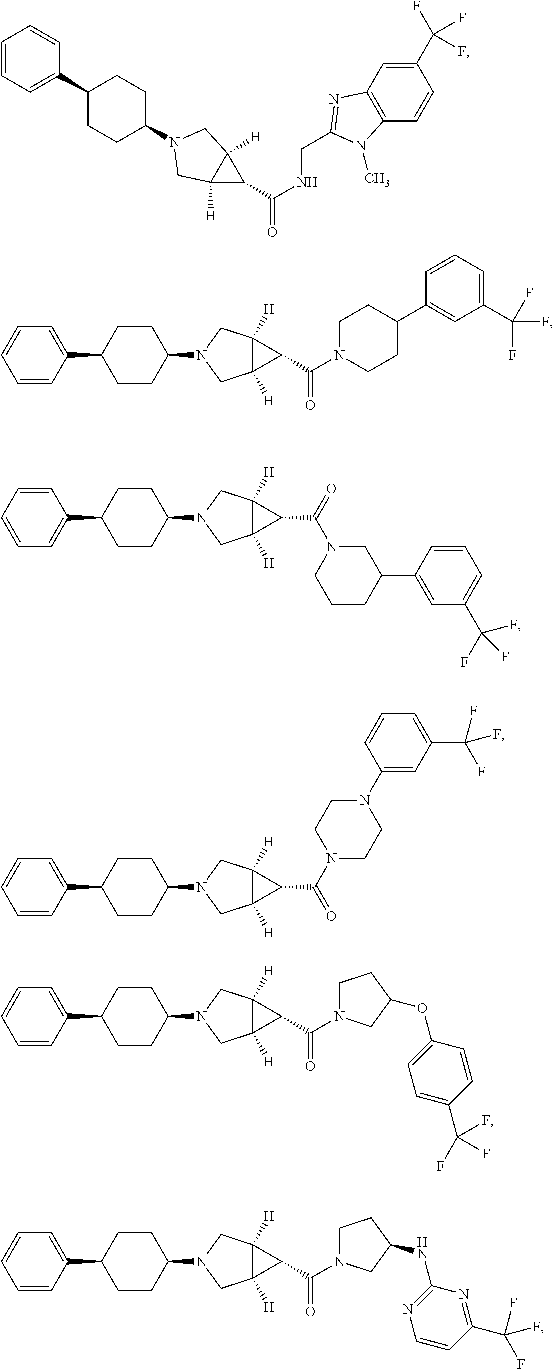 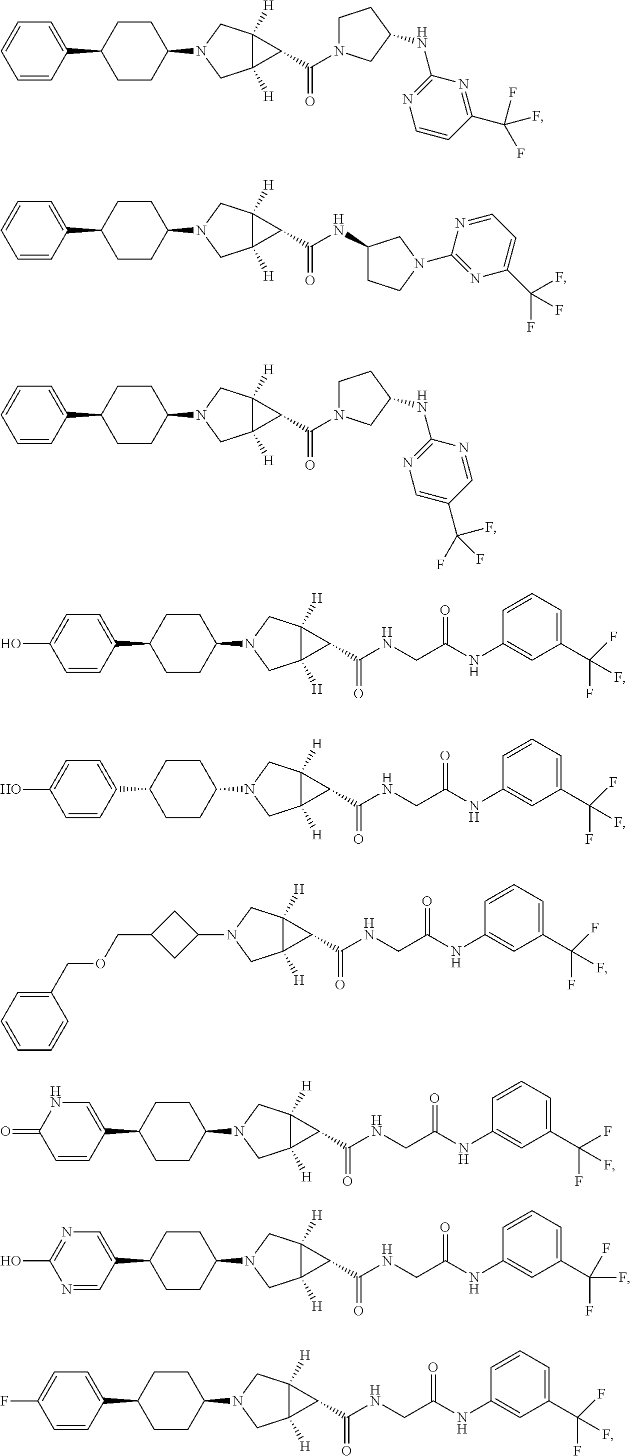 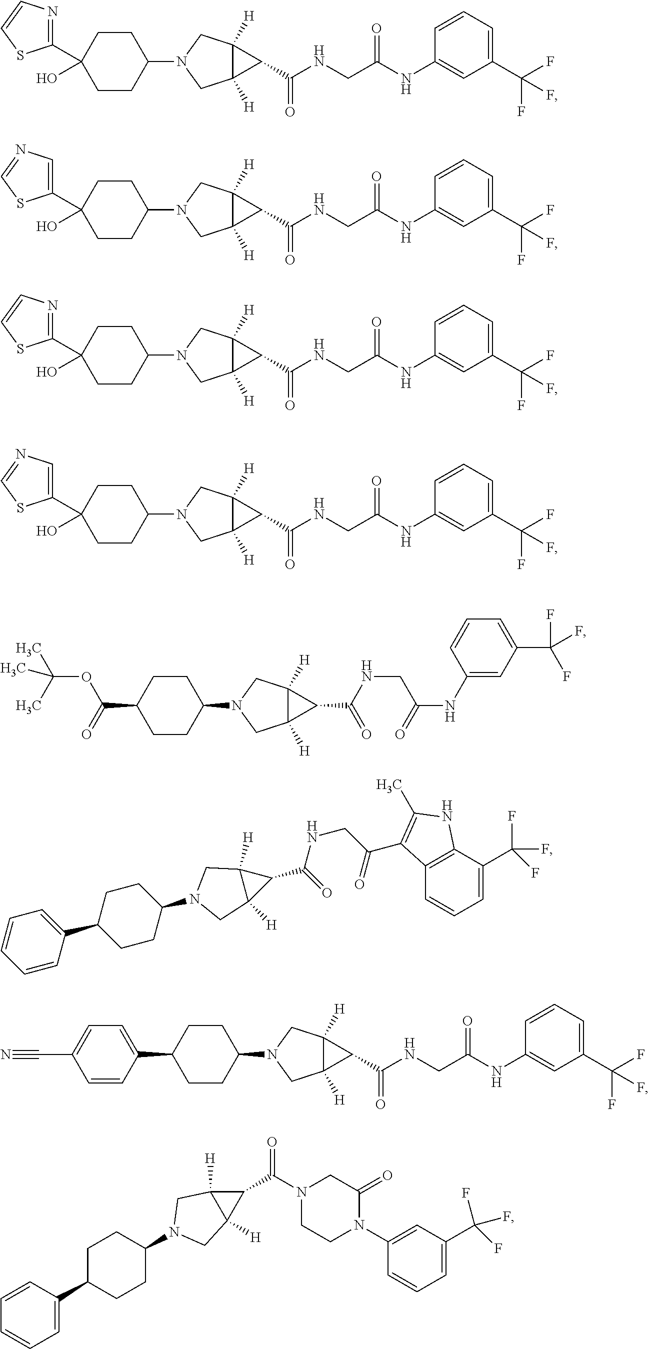 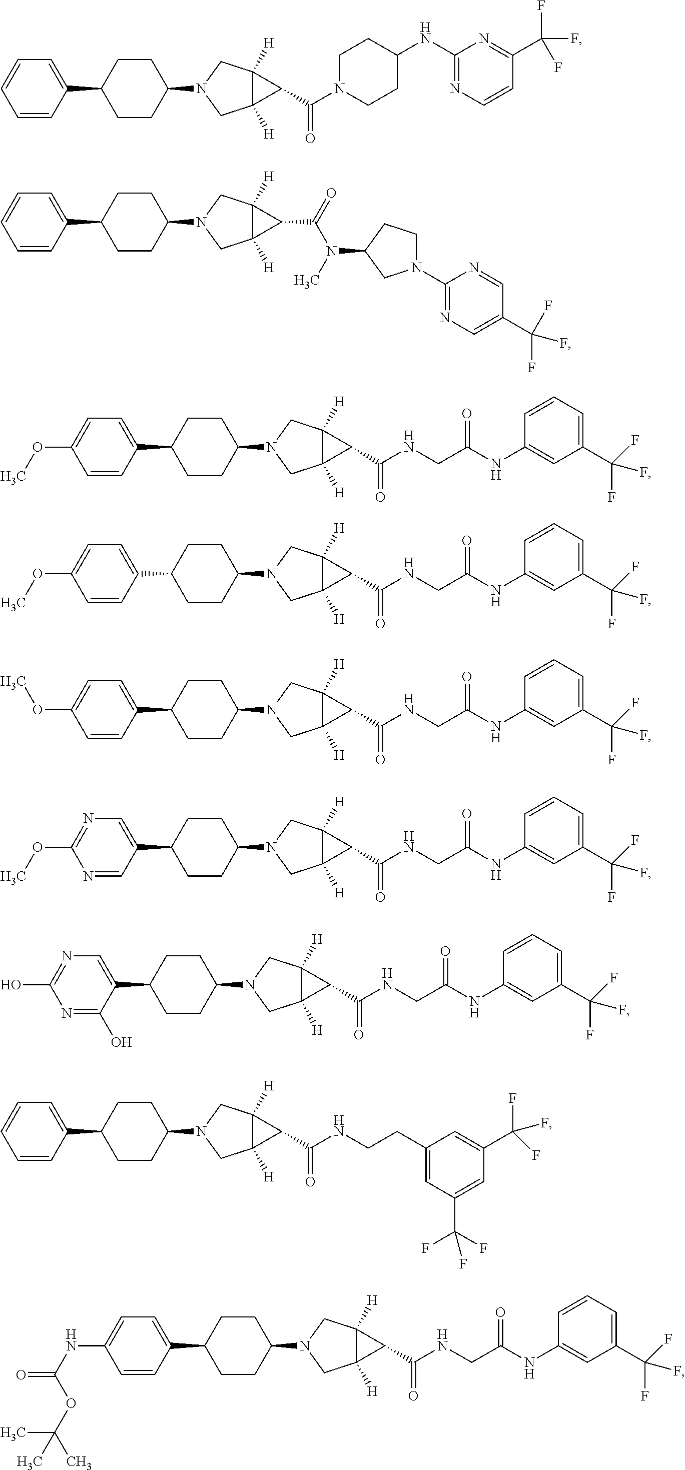 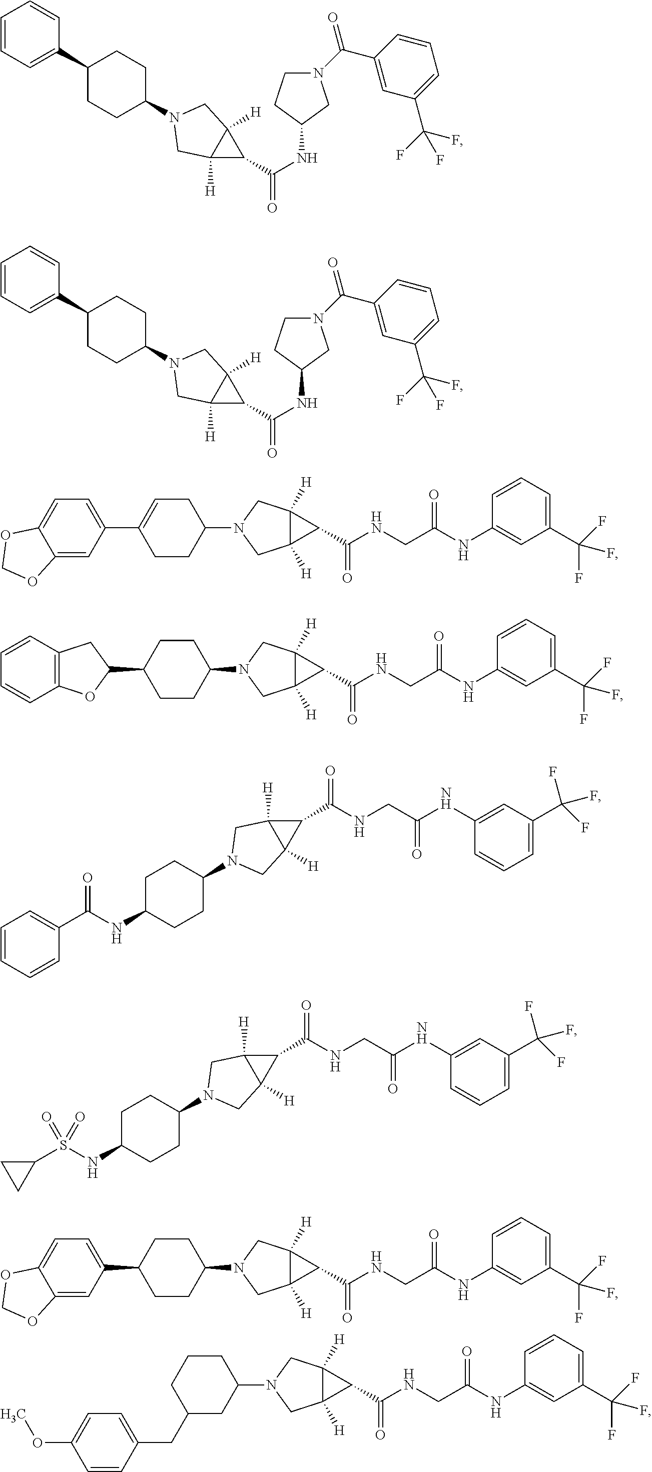 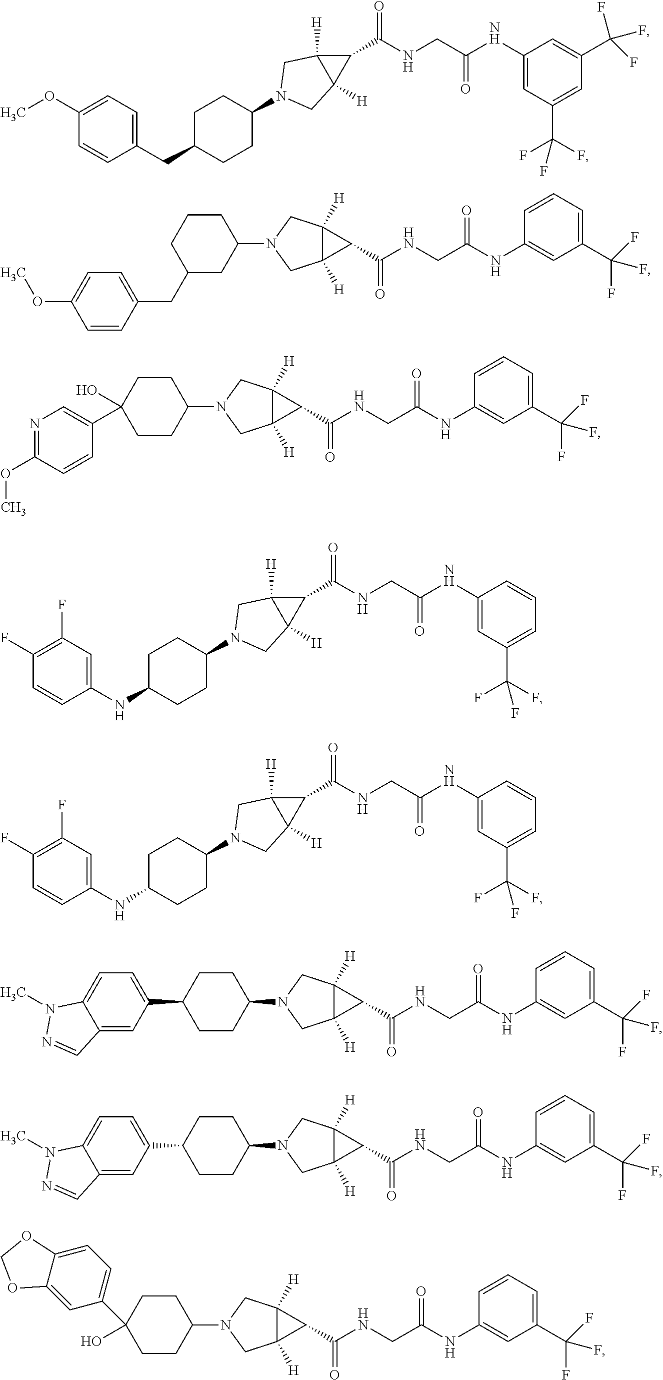 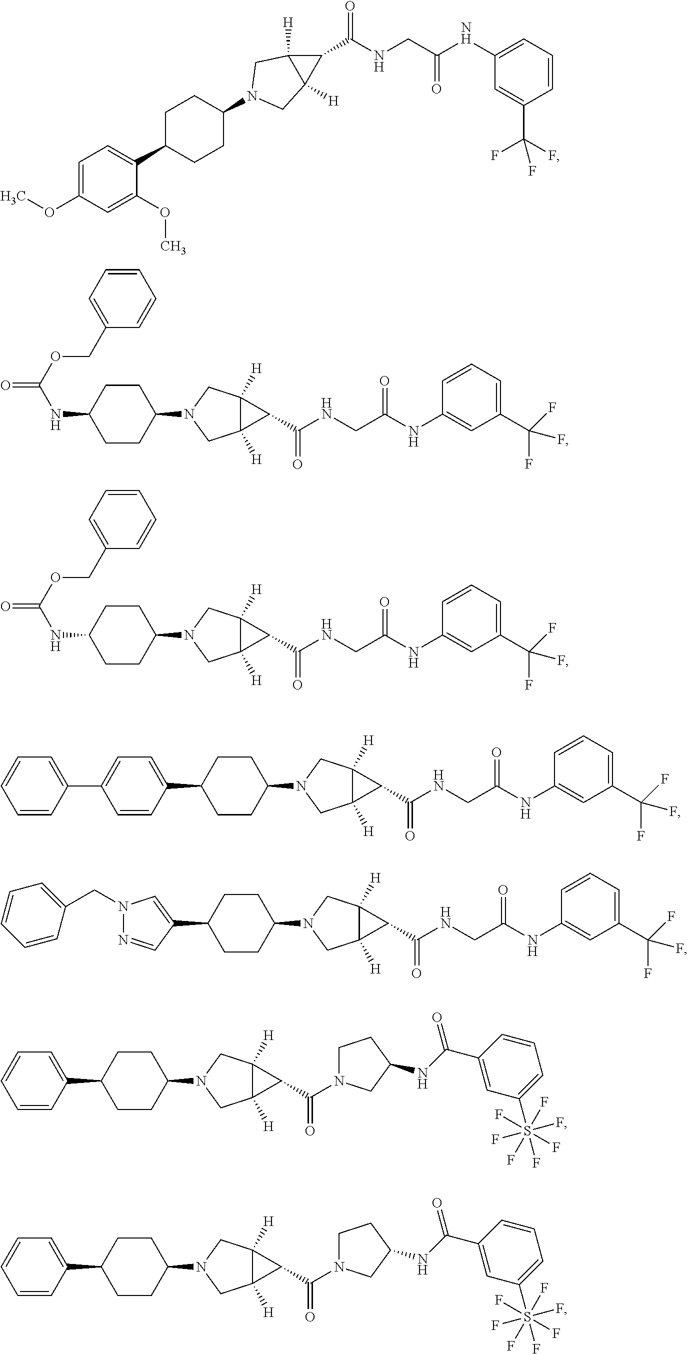 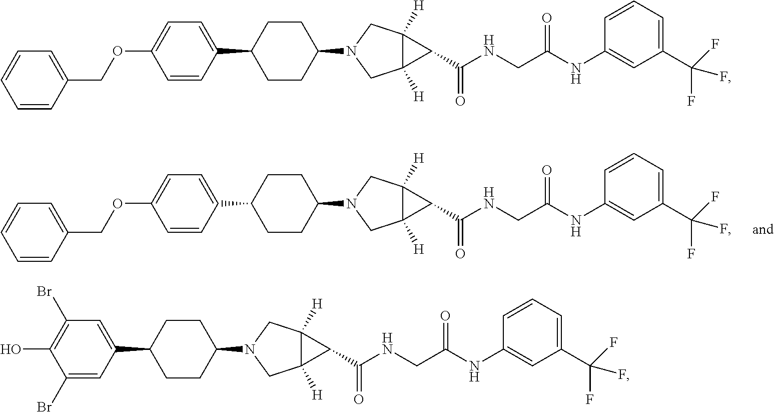
or a pharmaceutically acceptable salts thereof.

10. A pharmaceutical composition comprising at least one compound of claim 1 or a pharmaceutically acceptable salt thereof, and a pharmaceutically acceptable carrier.

11. A method for modulation of CCR2 receptor activity in a mammal which comprises the administration of an effective amount of the compound of claim 1.

12. A method for the treatment of an inflammatory and immunoregulatory disorder or disease which comprises the administration to a patient of an effective amount of the compound of claim 1.

13. A method for the treatment of rheumatoid arthritis which comprises the administration to a patient of an effective amount of the compound of claim 1.

14-17. (canceled)
